(19) United States
(12) Patent Application Publication
(10) Pub. No.: US 2018/0298370 A1
(43) Pub. Date: Oct. 18, 2018

(54) MODIFIED BACTERIAL NANOCELLULOSE AND ITS USES IN CHIP CARDS AND MEDICINE

(71) Applicant: Julius-Maximilians-Universität Würzburg, Würzburg (DE)

(72) Inventor: Thomas Dandekar, Würzburg (DE)

(21) Appl. No.: 15/569,600

(22) PCT Filed: Apr. 27, 2016
(86) PCT No.: PCT/EP2016/059436

§ 371 Date: Oct. 26, 2017

(30) Foreign Application Priority Data
Apr. 27, 2015 (DE) 102015005307.8
Apr. 27, 2015 (DE) 102015005308.6

(51) Int. Cl. C12N 11/12; A61K 35/36; A61K 35/28; A61K 47/38; A61L 27/44; A61L 27/54; A61L 15/28; A61L 15/36; C12N 9/12; C07K 14/195; C12N 15/70; C12Q 1/48; C08B 15/02; B33Y 70/00

(52) U.S. Cl. CPC C12N 11/12; B33Y 10/00; A61K 35/28; A61K 47/38; A61L 27/44; A61L 27/54; A61L 15/28; A61L 15/36; C12N 9/1205; C12Y 207/01078; C12N 9/1247; C12Y 207/07006; C07K 14/195; C12N 15/70; C12Q 1/485; C08B 15/02; B33Y 70/00; C07K 2319/60; A61K 35/36

(57) ABSTRACT

The present invention relates to bacterial nanocellulose composite which comprises nanocellulose, sensor or signal processing molecule(s), actuator/effector molecule(s) and/or cells and optionally further component(s). The present invention further relates to the use of the bacterial nanocellulose composite in chip technology and material engineering. The present invention relates to a printing, storage and/or processing medium as well as a smart card or chip card comprising the bacterial nanocellulose composite. The present invention further relates to the medical use of the bacterial nanocellulose composite, preferably in wound healing, tissue engineering and as transplant. The present invention further relates to a skin, tissue or neuro transplant. The present invention also relates to methods of stimulus conduction, muscle stimulation and/or monitoring heartbeat. The present invention further relates to a method for producing a nanocellulose composite chip using 3D printer.

The present invention relates to a bacterial nanocellulose composite which comprises nanocellulose, sensor or signal processing molecule(s), actuator/effector molecule(s) and/or cells and optionally further component(s). The present invention further relates to the use of the bacterial nanocellulose composite in chip technology and material engineering. The present invention relates to a printing, storage and/or processing medium as well as a smart card or chip card comprising the bacterial nanocellulose composite. The present invention further relates to the medical use of the bacterial nanocellulose composite, preferably in wound healing, tissue engineering and as transplant. The present invention further relates to a skin, tissue or neuro transplant. The present invention also relates to methods of stimulus conduction, muscle stimulation and/or monitoring heartbeat. The present invention further relates to a method for producing a nanocellulose composite chip using 3D printer.

### BACKGROUND OF THE INVENTION

Nanocellulose is a term referring to nano-structured cellulose. This can be cellulose nanofibers (CNF) also called microfibrillated cellulose (MFC), nanocrystalline cellulose (NCC), or bacterial nanocellulose (BNC), which refers to nano-structured cellulose produced by bacteria. Nanocellulose/CNF or NCC can be prepared from any cellulose source material, but woodpulp is normally used.

At the moment, nanocellulose is produced in increasing amounts worldwide. For example, Kralisch et al., 2015 describe a molecular biological method for bacterial nanocellulose production, also used by the company JeNaCell GmbH (Jena, Germany). At Edinburgh University and Sappi Limited (Johannesburg, South Africa) use an energy efficient macrocopic process for converting wood biomass into nanocellulose. In the US, the company American Process Inc. also uses biomass for the production of nanocellulose. In Mumbai (India), the ICAR-CIRCOT pilot plant produces daily 10 kg of nanocellulose since October 2014. Furthermore, the association of nonwoven fabrics industry which names nanocellulose as “the amazing material that promises flexible displays, faster cars and bullet-proof suits” focusses on the use of algae and sun light for the production of nanocellulose. See e.g. the association's congress “Rise 2015” (from graphene and nanofibers to intelligent fabrics and wearable electronics—at INDA's Research, Innovation & Science for Engineered Fabrics Conference (RISE®) and Nanofibers for the Third Millennium (N3M), February 9-12, in Miami, Fla.).

Nanocellulose is used in a plurality of applications, such as disclosed in US 2015/0024379 A1, US 2014/0370179 A1, US 2014/0367059 A1, US 2014/0345823 A1, US 2014/0323714 A1, US 2014/0323633 A1, US 2014/0224151 A1, US 2014/0255688 A1, US 2014/0088223 A1, US 2014/0202517 A1.

Furthermore, nanocellulose complements and replaces other materials used so far as biomatrices for tissue replacements. Such materials are e.g. synthetic materials, such as polyisopropyl acrylamid which in combination with polyethylene glycol polymerizes in the body due to the body temperature to a stabile bioadhesice matrix (Vernengo et al., 2010). There are biopolymers of chitosan, collagen, alginate, gelatin, elastin, fibrin, hyaluronic acid or silk protein, which are applied as beads, sponges, molded paddings, hydrogel or primarily in liquid form (Allen et al., 2004, Meakin 2001, Wilke et al., 2004, Sebastine and Williams 2007, Gruber et at, 2006). Matrices of atelocollagen are suitable for the cultivation of human mesenchymal stem cells (hMSC) (Sakai et al., 2005; Sakai et al., 2006; Lee et al., 2012). Scaffolds made of a combination of chitosan and gelatin provide suitable conditions for the cultivation of intervertebral disk cells isolated from rabbits (Cheng et al., 2010). Alginate obtained from brown algae is a suitable matrix for the cultivation of intervertebral disk cells as well (Chou et al., 2009).

All this confirms, there is a need in the art for improved nanocellulose materials to become an intelligent material that can process or store information. There is a need in the art for improved nanocellulose material which is suitable or can be tailored for a plurality of uses.

### SUMMARY OF THE INVENTION

According to the present invention this object is solved by a bacterial nanocellulose composite, said bacterial nanocellulose comprising apart from the nanocellulose matrix DNA or RNA or modified nucleotides or further components for information processing.

According to the present invention this object is solved by a bacterial nanocellulose composite, said bacterial nanocellulose comprising nanocellulose and
(i) sensor or signal processing molecule(s);
   preferably light-inducible or light-responding sensor or signal processing molecule(s),
   more preferably protein(s) or protein domain(s) comprising light-inducible or light-responding sensor domain(s),
and/or
(ii) actuator or effector molecule(s);
   optionally light-inducible or light-responding actuator or effector molecule(s), such as protein(s) or protein domain(s) comprising light-inducible or light-responding sensor domain(s),
and/or
(iii) cells;
(iv) optionally, further component(s).

According to the present invention this object is solved by using the bacterial nanocellulose composite of the present invention
in material engineering
in chip technology
as printing matrix or printed nanocellulose composite,
as transparent material or display or information processing device for LED and chips/chip technology,
as printing, storage and/or processing medium,
as detector,
as intelligent foil,
as intelligent material,
as nanofactory,
as sophisticated, light-controlled, synthesis device,
as small biochemical analyzer,
in DNA-based ASIC (application-specific chip) for sequence storage or analysis.

According to the present invention this object is solved by using the bacterial nanocellulose composite of the present invention
in wound healing and tissue engineering,
as skin transplant, band-aid or tissue implant,
as neuro transplant,
for stimulus conduction,
for muscle stimulation,
as electronic skin,
for monitoring wound healing, heartbeat, or other physical parameters,
for faster regeneration,
for reprogramming body cells during the healing process,
as intelligent plaster.

According to the present invention this object is solved by a printing, storage and/or processing medium comprising the bacterial nanocellulose composite of the present invention.

According to the present invention this object is solved by a smart card or a chip card comprising the bacterial nanocellulose composite of the present invention.

According to the present invention this object is solved by providing the bacterial nanocellulose composite of the present invention for use as a medicament.

According to the present invention this object is solved by providing the bacterial nanocellulose composite of the present invention for use in a method of treating wounds and/or for detecting wounds and wound healing and/or for monitoring wound healing.

According to the present invention this object is solved by providing the bacterial nanocellulose composite of the present invention for use in a method of tissue engineering.

According to the present invention this object is solved by a skin transplant, tissue implant or neuro transplant comprising the bacterial nanocellulose composite of the present invention.

According to the present invention this object is solved by providing the bacterial nanocellulose composite of the present invention for use in a method of stimulus conduction, muscle stimulation and/or for monitoring heartbeat.

According to the present invention this object is solved by electronic skin comprising the bacterial nanocellulose composite of the present invention.

According to the present invention this object is solved by a method for producing a nanocellulose composite chip, comprising the steps of
(1) providing a nanocellulose composite or providing nanocellulose or and the component(s) to be included in the nanocellulose,
(2) using a 3D printer or laser sintering, and
(3) obtaining the nanocellulose composite chip.

According to the present invention this object is solved by a nanocellulose composite chip obtained by the method of the present invention.

### DESCRIPTION OF THE PREFERRED EMBODIMENTS OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

Bacterial Nanocellulose Composite

Summary/Abstract

The present invention provides a bacterial nanocellulose composite, said bacterial nanocellulose composite comprising DNA or RNA or modified nucleotides or further components for information processing.

Specifically these are
(1) nanocellulose composite with light-gated nucleotide-specific polymerase constructs or other nucleotide processing or nucleotide binding enzymes (e.g. Cid I polymerase, μ-polymerases, exonucleases, transcription factors, T4 polynulceotide kinase, adenyltransferase) including light-gated versions of these enzymes or fluorescent protein constructs (GFP, YFP, CFP protein fusions) including light-gated versions for information storage and processing. Nucleotides (DNA, RNA) are used as substrate and synthesized nucleotides for storage.
(2) nanocellulose composite with light-gated RNA polymerases, or protein translation system or enzymatic synthesis system or enzymes or sensors or light-gated versions of these enzymes for molecular information processing of RNAs and proteins.
(3) nanocellulose composite with pores (from proteins or nucleic acid) for electronic or optical properties or fluorescent proteins or transparent nanocellulose or nanocellulose with modifyable optical properties or organic polymers or graphene or fullerene or dyes or sensor proteins or enzymes (active on the surface, active expressed at the surface, hence actuators for “printing”) as output device and for connection to electronic components (including typical refinement steps from chip manufacturing on the nanocellulose composite).
(4) nanocellulose composite with sensor proteins and/or modified nanocellulose surface (including fluorescent proteins, monitoring proteins or light-gated versions of these or dyes) for monitoring (e.g. in wounds).
(5) nanocellulose composite for programming cells with growth factors, or kinases or receptors or enzymes or drugs or light-gated versions (e.g. for intelligent plaster)
(6) nanocellulose composite containing cells, sensors or enzymes or pores, actuators or electronic parts to become part of a tissue (e.g. for an artificial skin).

As discussed above, the present invention provides bacterial nanocellulose composite materials.

Said bacterial nanocellulose composite comprises nanocellulose and
(i) sensor or signal processing molecule(s);
and/or
(ii) actuator or effector molecule(s);
and/or
(iii) cells;
(iv) optionally, further component(s).

The bacterial nanocellulose composite of the present invention can comprise one or more of each of the components (i) to (iii) (and optionally (iv) as well) and combinations of the components (i) to (iii), and optionally further component(s) (iv). The choice of the components will depend on the planned application of the bacterial nanocellulose composite, in particular molecular information processing.

The bacterial nanocellulose composite of the present invention can comprise at least one component (i);
at least one component (ii);
at least one component (iii);
components (i) and (ii) and (iii);
components (i) and (ii);
components (i) and ((iii);
components (ii) and (iii);
and optionally one or more further components (iv);

for example one or more of component (i);
one or more of component (ii);
one or more of component (iii);
one or more of each of components (i) and (ii) and (iii);
one or more of each of components (i) and (ii);
one or more of each of components (i) and ((iii);
one or more of each of components (ii) and (iii);
and optionally one or more further components (iv).

The term “bacterial nanocellulose” when used herein refers to nanocellulose made from bacteria, in particular with high grade, high purity and well controlled fibre size and structure. Plant made nanocellulose can only be used if it achieves similar high grade and properties as a nanocellulose composite.

The term “bacterial nanocellulose composite” when used herein refers to bacterial nanocellulose which comprises further components, as defined herein.

The bacterial nanocellulose composite of the present invention can comprise one or more of each of the components (i) to (iii) as well as combinations of the components (i) to (iii), and optionally further component(s). The choice of the components will depend on the planned application of the bacterial nanocellulose composite.

The term “sensor molecule” or “signal processing molecule” or “information processing molecule”—as interchangeably used herein—refers to a molecule or compound that senses a signal, such as light, temperature, ions, ligands, and/or electric current, and responds to the signal and/or processes the signal via a conformational change, an (enzymatic) reaction (such as DNA or RNA synthesis), translocation, and/or that transfers it to an actuator or effector.

The term “actuator” or “actuator molecule” or “effector molecule”—as interchangeably used herein—refers to a molecule or compound that further translates or processes or transmits the signal sensed and transferred from the sensor or signal processing molecule(s), such as via a conformational change, an (enzymatic) reaction (such as DNA or RNA synthesis), translation and protein expression.

In the application of the bacterial nanocellulose composite in chip technology, the “sensor molecule” or “signal processing molecule” is also referred to as “input”; and the “actuator molecule” or “effector molecule” is also referred to as “output”. The different substrates (further components (iv)) which are modified by both molecule types (further proteins, synthesis or degradation of nucleotides etc.) are referred to as “information processing” before the final output is created.

(i) Sensor or Signal Processing Molecules

The bacterial nanocellulose composite of the present invention can comprise at least one sensor or signal processing molecule.

As discussed above, the term “sensor molecule” or “signal processing molecule” or “information processing molecule”—as interchangeably used herein—refers to a molecule or compound that senses a signal, such as light, temperature, ions, ligands, and/or electric current, and responds to the signal and/or processes the signal via a conformational change, an (enzymatic) reaction (such as DNA or RNA synthesis), translocation, and/or that transfers it to an actuator or effector.

In the application of the bacterial nanocellulose composite in chip technology, the “sensor molecule” or “signal processing molecule” is also referred to as “input”.

Said sensor or signal processing molecule(s) is/are preferably light-inducible or light-responding sensor molecule(s), i.e. the signal is light.

The signal can also be temperature, ions, ligands, and/or electric current.

Said sensor or signal processing molecule(s) can be:
(a) protein(s) comprising light-inducible or light-responding sensor domain(s), or
(b) protein domain(s) fused to light-inducible or light-responding sensor domain(s),

Said proteins (a) can comprise said light-inducible or light-responding sensor domain(s) either naturally, or said proteins (a) are fusions with said domains, preferably genetically engineered.

Said protein domain(s) can be enzymatically active domains or binding domains.

Furthermore, domain(s) of different proteins can be part of a construct with said light-inducible or light-responding sensor domain(s).

Preferably, the protein(s) comprising light-inducible or light-responding sensor domains are selected from:
polymerase(s);
   such as DNA polymerase(s), RNA polymerase(s),
   e.g. T4 polynucleotide kinase
   Cid1 polymerase
   PolyU polymerase
   μ DNA polymerase
   terminal deoxyncleotidyl (TdT) polymerase,
adenyltransferase(s);
ion channel(s) or pore(s);
   such as a GABA channel, glutaminergic channel or porin or protein channel, or other pores and ion channels, see e.g. Buga et al., 2012
membrane protein(s);
   lipoproteins, glycoproteins,
   such as bacteriorhodopsin,
receptors,
   such as TNF receptors, see Fricke et al., 2014,
or domains thereof,
or combinations thereof.

In some embodiments, domain(s) of the above mentioned protein(s) are used, such as catalytic or enzymatically active domains and/or binding domains.

In one embodiment, the protein(s)/protein domain(s) comprising light-inducible or light-responding sensor domain(s) further comprise linker(s) and/or secretion signal(s) or signal peptide domain(s).

This e.g. allows for the protein(s) or protein domain(s) to locate to/to be transported, or the like, to certain positions within the fibres of the nanocellulose (composite).

The choice of the sensor molecule(s)/proteins will depend on the planned application of the bacterial nanocellulose composite.

For example: planned application as smart card/chip card or the like (DNA storage medium): Suitable proteins are nucleotide-specific polymerase constructs or other nucleotide processing and/or binding proteins/enzymes, such as DNA polymerase(s) and RNA polymerase(s), such as Cid1 polymerase, PolyU polymerase, μ DNA polymerase, terminal deoxyncleotidyl (TdT) polymerase, or active domains thereof.

Suitable sensor or signal processing molecule(s) are e.g.:
fusion of a BLUF domain and T4 polynucleotide kinase,
fusion of a BLUF domain and PolyU polymerase (with a histidine in the active site),
fusion of a BLUF domain and PolyU polymerase (with an asparagine in the active site instead of the histidine, transforming the polymerase into a polyA polymerase).

For example, for nucleotide-based information processing the sensor or signal processing molecule(s) (i.e. protein(s)) are
polymerase(s) and exonuclease(s);
   such as DNA polymerase(s), RNA polymerase(s),
   e.g. T4 polynucleotide kinase
   Cid1 polymerase
   PolyU polymerase
   μ DNA polymerase
   terminal deoxyncleotidyl (TdT) polymerase.

For Example: Planned Medical Application

For e.g. the application as an “intelligent” nanocellulose composite for medical applications (such as, intelligent plaster) suitable sensor or signal processing molecule(s) are embodied in the intelligent nanocellulose composite an can monitor the state of the wound, e.g. measure temperature, pH, inflammation (cytokines) and can also show by a change in fluorescence the resulting state.

Furthermore, the healing process should be improved by suitable programming the tissue or cells. For this the nanocellulose composite can contain as further component(s) growth promoting molecules such as growth factors (VEGF, EGF, PDGF), kinases, but also connective tissue stimulating components such as collagens. All these different components are well controlled, monitored and only selectively released in the nanocellulose composite including a suitable surface treatment of the nanocellulose (iii).

(ii) Actuator Molecules

The bacterial nanocellulose composite of the present invention can comprise at least one actuator or effector molecule.

As discussed above, the term “actuator” or “actuator molecule” or “effector molecule”—as interchangeably used herein—refers to a molecule or compound that further translates or processes or transmits the signal sensed and transferred from the sensor or signal processing molecule(s), such as via a conformational change, an (enzymatic) reaction (such as DNA or RNA synthesis), translation and protein expression.

In the application of the bacterial nanocellulose composite in chip technology, the “actuator molecule” or “effector molecule” is also referred to as “output”.

This embodiment is particularly suitable for uses in chip technology and as storage medium.

Preferably, the actuator or effector molecule(s) are enzymes or structure proteins so that an output or action is transmitted to the nanocellulose surface

Said actuator or effector molecule(s) can be light-inducible or light-responding molecule(s), i.e. the signal is light.

Said actuator or effector molecule(s) can be:
(a) protein(s) comprising light-inducible or light-responding sensor domain(s), or
(b) protein domain(s) fused to light-inducible or light-responding sensor domain(s),

Said proteins (a) can comprise said light-inducible or light-responding sensor domain(s) either naturally, or said proteins (a) are fusions with said domains, preferably genetically engineered.

Preferably, the actuator or effector molecule(s) comprise light-inducible or light-responding domain(s)/protein(s) that respond to a light of different wavelength than the sensor or signal processing molecule(s).

In such embodiment, the molecules can be controlled individually from each other by the use of light of said different wavelengths.

Said protein domain(s) can be enzymatically active domains or binding domains.

Furthermore, domain(s) of different proteins can be part of a construct with said light-inducible or light-responding actuator or effector domain(s).

Preferably, the actuator or effector molecule(s)/protein(s) (optionally comprising light-inducible or light-responding sensor domains) are selected from:
polymerase(s);
   such as DNA polymerase(s), RNA polymerase(s),
exonuclease(s);
transcription factor(s);
nucleotide binding domain(s);
enzyme(s);
structural protein(s);
protein translation enzyme(s);
or domains thereof,
or combinations thereof.

In some embodiments, domain(s) of the above mentioned protein(s) are used, such as catalytic or enzymatically active domains and/or binding domains.

The choice of the actuator or effector molecule(s)/protein(s) will depend on the planned application of the bacterial nanocellulose composite.

In one embodiment, the protein(s)/protein domain(s) comprising light-inducible or light-responding domain(s) further comprise linker(s) and/or secretion signal(s) or signal peptide domain(s).

This e.g. allows for the protein(s) or protein domain(s) to locate to/to be transported, or the like, to certain positions within the fibres of the nanocellulose (composite).

Light Inducible or Light-Responding Domains

Preferably, the light-inducible or light-responding sensor molecule(s) or light-inducible or the light-responding sensor/actuator/effecor domain(s) comprise or are:
Blue Light Using FAD domain (BLUF domain),
   such as BLUF domain of PAC protein of Euglena gracilis, Slr1694 of Synechocystis sp., AppA protein of Rhodobacter sphaeroides, Blrp of E. coli, from Klebsiella pneumoniae, Naegleria gruberi, Acinetobacter bayli,
Light-Oxygen Voltage sensing domain (LOV domain),
   such as LOV (light, oxygen, or voltage) domains of the blue-light photoreceptor phototropin (nph1) or LOV2-Jα,
   e.g. LOV1, LOV2,

or
cryptochromes (CRYs).

The BLUF domain (sensors of blue-light using FAD) is a FAD-binding protein domain. The BLUF domain is present in various proteins, primarily from bacteria, for example a BLUF domain is found at the N-terminus of the AppA protein from Rhodobacter sphaeroides. The BLUF domain is involved in sensing blue-light (and possibly redox) using FAD and is similar to the flavin-binding PAS domains and cryptochromes. The predicted secondary structure reveals that the BLUF domain has a novel FAD-binding fold.

BLUF-domain (the sensor for Blue Light Using FAD) is a novel blue light photoreceptor, identified in 2002 and it is found in more than 50 different proteins. These proteins are involved in various functions, such as photophobic responses (e.g. PAC protein—Euglena gracilis, Gomelsky and Klug, 2002; Slr1694—Synechocystis sp. Okajima et al., 2005) and regulation of transcription (e.g. AppA protein—Rhodobacter sphaeroides, Masuda and Bauer, 2005; Blrp—E. coli, Pesavento and Hengge, 2009). The proteins containing BLUF or similar domain was found also in Klebsiella pneumonia (Tyagi et al., 2013), Naegleria gruberi (Yasukawa et al., 2013), Acinetobacter baylyi (Bitrian et al., 2013) and many others organism. The molecular mechanism of BLUF-domain is very sophisticated. It converts the light signal to the biological information, following the conformational changes of the photoreceptor. Those changes are then recognized by other protein modules that transmit the signal to the downstream machineries. This type of light signal transduction mechanism was specifically modified in each organism during the evolution, to allow the adaptation for the different environmental conditions.

The BLUF domain can in particular be obtained as part of the YcgF gene and protein (Tschwori et al., 2009; Tschwori et al., 2012). DNA for the BLUF domain can, thus, in particular be gene ycgF (Accession number AAC74247.3) from E. coli.

See e.g. SEQ ID NO. 1, as listed in Database: UniProt/SWISS-PROT, Entry: BLUF_ECOLI

```
SEQUENCE 403 AA
          MLTTLIYRSH IRDDEPVKKI EEMVSIANRR NMQSDVTGIL
          LFNGSHFFQL LEGPEEQVKM IYRAICQDPR HYNIVELLCD
          YAPARRFGKA GMELFDLRLH ERDDVLQAVF DKGTSKFQLT
          YDDRALQFFR TFVLATEQST YFEIPAEDSW LFIADGSDKE
          LDSCALSPTI NDHFAFHPIV DPLSRRIIAF EAIVQKNEDS
          PSAIAVGQRK DGEIYTADLK SKALAFTMAH ALELGDKMIS
          INLLPMTLVN EPDAVSFLLN EIKANALVPE QIIVEFTESE
          VISRFDEFAE AIKSLKAAGI SVAIDHEGAG FAGLLLLSRF
          QPDRIKISQE LITNVHKSGP RQAIIQAIIK CCTSLEIQVS
          AMGVATPEEW MWLESAGIEM FQGDLFAKAK LNGIPSIAWP
          EKK
```

Light-oxygen-voltage-sensing (LOV) domains are protein sensors used by a large variety of higher plants, microalgae, fungi and bacteria to sense environmental conditions. In higher plants, they are used to control phototropism, chloroplast relocation, and stomatal opening, whereas in fungal organisms, they are used for adjusting the circadian temporal organization of the cells to the daily and seasonal periods. Common to all LOV proteins is the blue-light sensitive flavin chromophore, which in the signaling state is covalently linked to the protein core via an adjacent cysteine residue. LOV domains (Mart et al., 2016) are e.g. encountered in phototropins, which are blue-light-sensitive protein complexes regulating a great diversity of biological processes in higher plants (e.g. phototropin 2 in Arabidopsis thaliana, genbank accession CP002688.1) as well as in micro-algae.

Phototropins are composed of two LOV domains, each containing a non-covalently bound flavin mononucleotide (FMN) chromophore in its dark-state form, and a C-terminal Ser-Thr kinase. Upon blue-light absorption, a covalent bond between the FMN chromophore and an adjacent reactive cysteine residue of the apo-protein is formed in the LOV2 domain (Yao et al., 2008). This subsequently mediates the activation of the kinase, which induces a signal in the organism through phototropin autophosphorylation. In case of the fungus Neurospora crassa, the circadian clock is controlled by two light-sensitive domains, known as the white-collar-complex (WCC) and the LOV domain vivid (VVD-LOV). LOV domains have also been found to control gene expression through DNA binding and to be involved in redox-dependent regulation, like e.g. in the bacterium Rhodobacter sphaeroides.

Furthermore, the crystal structure of Lov1 Domain for instance of Phototropin2 from Arabidopsis thaliana (PDB code 2Z6D_B) is known in atomic detail (e.g. allowing an easier engineering, such as these for light-dependend control of the subsequent CidI polymerase, see below).

Amino Acid Sequence of Lov1 Domain:

| SEQ ID NO. 8 | |
|---|---|
| 1 | fprvsqelkt alstlqqtfv vsdatqphcp ivyassgfft mtgysskeiv grncrflqgp |
| 61 | dtdknevaki rdcvkngksy cgrllnykkd gtpfwnlltv tpikddqgnt ikfigmqvev |
| 121 | skytegvndk |

Cryptochromes (CRYs) are a class of flavoproteins that are sensitive to blue light. They are found in plants and animals. Cryptochromes are involved in the circadian rhythms of plants and animals, and in the sensing of magnetic fields in a number of species.

The two genes Cry1 and Cry2 code for the two cryptochrome proteins CRY1 and CRY2. In insects and plants, CRY1 regulates the circadian clock in a light-dependent fashion, whereas, in mammals, CRY1 and CRY2 act as light-independent inhibitors of CLOCK-BMAL1 components of the circadian clock. In plants, blue light photoreception can be used to cue developmental signals.

Examples of fusion protein constructs of BLUF domains with polymerases or domains of polymerases are disclosed in German patent application of one of the inventors, DE 10 2013 004 584.3, which is enclosed herewith in its entirety.

Such examples are for instance:
fusion of a BLUF domain and T4 polynucleotide kinase,
fusion of a BLUF domain and PolyU polymerase (with a histidine in the active site),
fusion of a BLUF domain and PolyU polymerase (with an asparagine in the active site).

Fluorescent Proteins and Protein Domains

In one embodiment, the sensor molecule(s) (i) and/or the actuator or effector molecule(s) (ii) comprise or are
(a) fluorescent protein(s),
(b) protein(s) or protein domain(s) comprising fluorescent domain(s), or
(c) fusions of protein(s) or protein domain(s) with fluorescent protein(s) or fluorescent domain(s),

In one embodiment, the fluorescent protein(s) or protein(s) comprising fluorescent domain(s) or fusion protein(s) with fluorescent protein(s) or domain(s) comprise
GFP, CFP, YFP,
or other fluorescent protein(s)/domain(s), such as

Embodiment where (i) and (ii) are Combined

In one embodiment, the sensor or signal processing molecule (i) and the actuator or effector molecule (ii) can be combined in one molecule or can be fused to each other.

For example,
a GFP-tagged sensor, in particular suitable in a nanocellulose plaster.
two component systems composed of sensors and actuators/responders as known from various bacteria; such as described in Krüger et al., 2012.

(iii) Cells

The bacterial nanocellulose composite of the present invention can comprise cells.

This embodiment is particularly suitable for medical uses.

Examples for cells are skin cells, stem cells (such as mesenchymal stem cells).

For example, the bacterial nanocellulose composite can comprise mesenchymal stem cells when it is to be used in wound healing.

For example, the bacterial nanocellulose composite can comprise specific tissue cells when it is to be used in tissue engineering, such as artificial lung tissue cells (see e.g. Stratmann et al., 2014)

(iv) Further Components

The bacterial nanocellulose composite of the present invention can comprise further component(s).

Said further component(s) can be components for the sensor/actuator/effector molecule(s).

For example:
nucleic acid(s) (e.g. DNA, RNA),
(oligo)nucleotide(s),
modified nucleotide(s),
enzyme substrate(s),
cofactor(s),
ion(s),
metabolite(s),
receptor ligand(s),
or combinations thereof.

Said further component(s) can be further polymer(s).

For example:
organic polymer(s),
poly nitrocellulose,
silicone (with or without polysinalisation),
or combinations thereof.

Said further component(s) can be graphene or fullerene.

Graphene, for instance, serves better interfacing with electronic components.

Said further component(s) can also be marker(s), label(s).

For example: chromophores, fluorophores and/or radioisotopes.

They can, for instance, serve to enhance clarity of the output on the surface of the nanocellulose composite.

Said further component(s) can also be compounds supporting wound healing and/or stimulating (tissue) growth.

For example:
growth factors and hormones, e.g. VEGF, erythropoietin, EGF, PDGF,
structural proteins, e.g. collagen I, II, X, aggrecan,
matrix-degenerating proteins, e.g. MMP-2,
or combinations thereof.

Said further component(s) can also be drugs, antibodies or antibody fragments.

The bacterial nanocellulose composite of the present invention can comprise combinations of said further component(s),

such as enzyme substrate(s) and cofactor(s) and ion(s),

such as (oligo)nucleotide(s) and further polymer(s),

and so on.

Nanocellulose Composite with Surface or Surface Layer

In one embodiment, the bacterial nanocellulose composite of the present invention forms or comprises a surface or surface layer.

Said surface or surface layer preferably comprises sensor or signal processing molecule(s) (i) which can be selected from:
ion channel(s) or pore(s);
   such as a GABA channel, glutaminergic channel, porin or protein channel,
membrane protein(s), lipoprotein(s), glycoproteins;
   such as bacteriorhodopsin,
receptor(s)
   such as TNF receptors, (see e.g. Fricke et al., 2014)
enzymes, which are preferably active on the surface,
or combinations thereof.

For example, said sensor proteins or enzymes are active on the surface and/or active expressed at the surface, hence actuators for “printing”. One example are two component systems composed of sensors and actuators/responders as known from various bacteria; described in e.g. Krüger et al., 2012.

In one embodiment, said surface or surface layer optionally comprises further component(s), such as
fluorescent proteins,
transparent nanocellulose or nanocellulose with modifyable optical properties,
organic polymers,
graphene or fullerene,
or dyes,
or combinations thereof.

These embodiments provide a nanocellulose composite with a surface suitable for electronic or optical properties to interface to electronic components or achieve output.

Thereby, the nanocellulose composite provides a natural surface. Modifying the surface by pores or modification of the nanocellulose itself yields electronic properties or provides optical properties. The nanocellulose composite for information processing can now use these optical and electronical properties for displaying the stored information (e.g. by fluorescence) or for interfacing electronically or optically with other electronic devices (e.g. smart phone, computer, glass-fibre cable).

Methods of Obtaining the Bacterial Nanocellulose and the Composite

Preferably, the bacterial nanocellulose is obtained via bacterial fermentation or bacterial expression.

For example, in
gram-negative bacteria, such as E. coli,
Komagataeibacter (named previously as Acetobacter or Gluconacetobacter)
Cyanobacteria.

The bacterial nanocellulose can be obtained from plant sources and is then bacterially fermented.

For example, according to Kralisch et al. (2014) Komagataeibacter (previous name: Acetobacter or Gluconacetobacter) is used.

Growth medium: Hestrin-Schramm medium made from water, glucose, yeast extract plus pepton, pH buffering—wherein numerous alternative media, for instance from plants are known.

One advantage of the procedure according to Kralisch et al. is the obtainment of high quality bacterial nanocellulose on the surface of the culture with a continuous process for constant and efficient production of nanocellulose.

For example, according to Nobles and Brown (2008) cyanobacteria, in particular Synechococcus leopoliensis strain UTCC 100, are used.

Transfer of the nanocellulose synthesis into cyano bacteria can enhance the yield. Nanocellulose is generated in a bioreactor at moderate temperatures (25-30° C.) at the surface of the liquid culture (interface to air) as a structure stable hydro-polymer (solid phase fraction about 1%, hydrogel). The polymer is harvested at the surface.

From a molecular perspective, nanocellulose is generated between cell wall and external membrane of the bacterial cell by a cellulose synthase complex which produces nanocellulose as a quite long glucose chain molecule from UDP-glucose monomers. The glucose polymers leave the cell as cellulose elementary fibrils through pores at the surface and aggregate to microfibrils. This self-assembly together with cell division and branching resulting therefrom, leads to the characteristic three dimensional fiber network.

According to the present invention, the production of the bacterial nanocellulose (composite) relies on expression in E. coli. For details, see the examples. The described method allows an easy production as well as manipulation of the nanocellulose and the resulting nanocellulose composite.

There are different ways for “adding” or including or embedding the components (i) to (iv) to/into the bacterial nanocellulose:

In one embodiment, the sensor or signal processing molecule(s) (i) and/or actuator/effector molecule(s) (ii) and/or cell(s) (iii) and further component(s), if present, are embedded or encapsulated in the bacterial nanocellulose composite.

In this embodiment, the component(s) can be added to the bacterial nanocellulose.

The sensor or signal processing molecule(s) (i) and/or actuator/effector molecule(s) (ii) and/or further component(s) (iv), if present, can also be co-produced during the bacterial fermentation or bacterial expression of the bacterial nanocellulose itself.

Thereby, particular expression constructs and cell biological cell lines are utilized.

In one embodiment, the sensor molecule(s) (i) and/or actuator/effector molecule(s) (ii) and/or further component(s) (iv), if present, are covalently attached to the nanocellulose,
such as via linker (e.g. nucleotide or peptide linker), anchor groups or cantilever.

In this embodiment, the bacterial nanocellulose and/or the sensor/actuator/effector molecule(s)/further component(s) can comprise said linker, anchor groups.

The component(s) can be added to the bacterial nanocellulose or they can also be co-produced during the bacterial fermentation or bacterial expression of the bacterial nanocellulose itself. Thereby, particular expression constructs and cell biological cell lines are utilized.

In one embodiment, which comprises more than one of the components (i) to (iii) and optionally further component(s) (iv), one or more of said component(s) can be embedded or encapsulated whereas one or more of said components can be covalently attached.

The skilled artisan will be able to choose the most suitable way, dependent on the planned application/use of the bacterial nanocellulose composite.

Uses of the Bacterial Nanocellulose Composite

As discussed above, the present invention provides the use of the bacterial nanocellulose composite in material engineering and chip technology.

In particular, the present invention provides the use of the bacterial nanocellulose composite
in material engineering
in chip technology
as printing matrix or printed nanocellulose composite,
   such as in 3D printing,
as transparent material or display or information processing device for LED and chips/chip technology,
   such as smart card, computer chip or chip card,
as printing, storage and/or processing medium,
as detector,
as intelligent foil,
as intelligent material,
as nanofactory,
as sophisticated, light-controlled, synthesis device,
as small biochemical analyzer,
in DNA-based ASIC (application-specific chip) for sequence storage or analysis.

As discussed above, the present invention provides the use of the bacterial nanocellulose composite in wound healing and tissue engineering.

In particular, the present invention provides the use of the bacterial nanocellulose composite
as material in wound healing and tissue engineering,
as skin transplant, band-aid or tissue implant,
as neuro transplant,
for stimulus conduction,
for muscle stimulation,
as electronic skin,
for monitoring wound healing, heartbeat, or other physical parameters,
for faster regeneration,
for reprogramming body cells during the healing process,
as intelligent plaster.

Preferably, the bacterial nanocellulose composite is used in form of a hydrogel, a foil, a layer, optical transparent paper.

Depending on the intended use, the composition of the nanocellulose composite changes, i.e. the components (i) to (iii) and optionally (iv) have to be chosen/combined.

For example:

(1) For Use in Information Storage and Processing: The nanocellulose composite of the present invention preferably comprises at least:
(i) light-gated nucleotide-specific polymerase constructs or other nucleotide processing or nucleotide binding enzymes (e.g. Cid I polymerase, mu-polymerases, exonucleases, transcription factors, 14 polynulceotide kinase, adenyltransferase)
   including light-gated versions of these enzymes or fluorescent protein constructs (GFP, YFP, CFP protein fusions)
(iv) nucleotides (DNA, RNA) are used as substrate,

The synthesized nucleotides are for storage.

(2) For molecular information processing of RNAs and proteins:

The nanocellulose composite of the present invention preferably comprises at least:
(i) light-gated RNA polymerases, or
protein translation system or enzymatic synthesis system or enzymes or sensors
   including light-gated versions of these enzymes for molecular information processing of RNAs and proteins.

(3) As output device and for connection to/interfacing with electronic components

The nanocellulose composite of the present invention preferably comprises at least:
(i) pores (from proteins or nucleic acid) for electronic or optical properties, or fluorescent proteins,
(iv) transparent nanocellulose
or nanocellulose with modifyable optical properties
or organic polymers
or graphene or fullerene
or dyes
or sensor proteins or enzymes (active on the surface, active expressed at the surface, hence actuators for “printing”)

as output device and for connection to electronic components (including typical refinement steps from chip manufacturing on the nanocellulose composite).

(4) For monitoring, e.g. wounds or wound healing

The nanocellulose composite of the present invention preferably comprises at least:
(i) sensor proteins
and/or modified nanocellulose surface
   including fluorescent proteins, monitoring proteins or light-gated versions of these or
(iv) dyes

for monitoring (e.g. in wounds).

(5) For reprogramming wounds for optimal healing

The nanocellulose composite of the present invention preferably comprises at least:
(i) kinases or receptors or enzymes,
   including light-gated versions
(iv) growth factors, or drugs,
   including light-gated versions

(e.g. for intelligent plaster)

(6) As intelligent skin or tissue substitute

The nanocellulose composite of the present invention preferably comprises at least:
(iii) cells,
(i) sensors or enzymes or pores,
(ii) actuators or
(iv) electronic parts

to become part of a tissue (e.g. for an artificial skin).

Uses in Material Engineering and Chip Technology

As discussed above, the present invention provides a printing, storage and/or processing medium comprising the bacterial nanocellulose composite of the present invention.

Said medium is preferably in form of a foil or a transparent display.

As discussed above, the present invention provides a smart card or a chip card comprising the bacterial nanocellulose composite of the present invention.

Said smart card or chip card optionally further comprises graphene and/or organic polymer(s).

Preferably, the bacterial nanocellulose composite is in the form of a hydrogel in the inside of the smart card or the chip card, preferably with a solid nanocellulose surface.

Medical Uses

As discussed above, the present invention provides the bacterial nanocellulose composite for use as a medicament.

As discussed above, the present invention provides the bacterial nanocellulose composite for use in a method of treating wounds.

As discussed above, the present invention provides the bacterial nanocellulose composite for use in detecting wounds and wound healing.

As discussed above, the present invention provides the bacterial nanocellulose composite for use in a method of monitoring wound healing.

In said method of treating wounds and/or for detecting wounds and wound healing and/or for monitoring wound healing, the bacterial nanocellulose composite preferably comprises
cells,
   such as mesenchymal stem cells,
compound(s) supporting wound healing and/or stimulating growth,
   such as
      growth factors and hormones, e.g. VEGF, erythropoietin, EGF structural proteins, e.g. collagen I, II, X, aggrecan, matrix-degenerating proteins, e.g. MMP-2,
and/or marker(s) or label(s).

The bacterial nanocellulose composite is preferably a hydrogel.

As discussed above, the present invention provides the bacterial nanocellulose composite for use in a method of tissue engineering.

In said method, the bacterial nanocellulose composite preferably comprises
cells,
growth factors,
structural proteins,
optionally, markers or labels.

As discussed above, the present invention provides a skin transplant, tissue implant or neuro transplant comprising the bacterial nanocellulose composite of the present invention.

As discussed above, the present invention provides a tissue implant comprising the bacterial nanocellulose composite of the present invention.

As discussed above, the present invention provides a neuro transplant comprising the bacterial nanocellulose composite of the present invention.

Combined Uses

As discussed above, the present invention provides the bacterial nanocellulose composite for use in a method of stimulus conduction, muscle stimulation and/or for monitoring heartbeat.

In said method, the bacterial nanocellulose composite preferably comprises
(i) sensor or signal processing molecule(s),
(ii) actuator or effector molecule(s),
(iii) cells,

preferably mesenchymal stem cells,
(iv) further components
   compounds supporting wound healing and/or stimulating growth
      preferably
      growth factors and hormones, e.g. VEGF, erythropoietin, EGF structural proteins, e.g. collagen I, II, X, aggrecan, matrix-degenerating proteins, e.g. MMP-2
      and/or marker(s) or label(s).

As discussed above, the present invention provides an electronic skin comprising the bacterial nanocellulose composite of the present invention.

Wound Healing and Tissue Engineering Methods

(1) The present invention provides a method of treating wounds.

Said method comprises the step of administering to a wound of a subject in need thereof a therapeutically active amount of the bacterial nanocellulose composite of the present invention.

(2) The present invention provides a method for detecting wounds and wound healing and/or for monitoring wound healing.

Said method comprises the step of administering to a wound of a subject in need thereof the bacterial nanocellulose composite of the present invention.

In above methods (1) and (2), the bacterial nanocellulose composite preferably comprises
cells,
   such as mesenchymal stem cells,
compound(s) supporting wound healing and/or stimulating growth,
   such as
      growth factors and hormones, e.g. VEGF, erythropoietin, EGF structural proteins, e.g. collagen I, II, X, aggrecan, matrix-degenerating proteins, e.g. MMP-2
and/or marker(s) or label(s),

The bacterial nanocellulose composite is preferably a hydrogel.

(3) The present invention provides a method of tissue engineering.

Said method can be an in vitro, ex vivo or in vivo method.

Said method (3) comprises the use of the bacterial nanocellulose composite of the present invention, which preferably comprises
cells,
growth factors,
structural proteins,
optionally, markers or labels.

(4) The present invention further provides a method of stimulus conduction, muscle stimulation and/or for monitoring heartbeat.

Said method (4) comprises the step of administering to a subject in need thereof the bacterial nanocellulose composite of the present invention.

The bacterial nanocellulose composite preferably comprises
(i) sensor or signal processing molecule(s),
(ii) actuator or effector molecule(s),
(iii) cells,
   preferably mesenchymal stem cells,
(iv) further components
   compounds supporting wound healing and/or stimulating growth,
      preferably
      growth factors and hormones, e.g. VEGF, erythropoietin, EGF
      structural proteins, e.g. collagen I, II, X, aggrecan,
      matrix-degenerating proteins, e.g. MMP-2
   and/or marker(s) or label(s).

3D Printing Method

As discussed above, the present invention provides a method for producing a nanocellulose composite chip.

Said method comprises the steps of
(1) providing a nanocellulose composite, preferably as defined herein, or providing nanocellulose or and the component(s) to be included in the nanocellulose, preferably as defined herein,
(2) using a 3D printer or laser sintering, and
(3) obtaining the nanocellulose composite chip.

Preferably, the nanocellulose in step (1) is
bacterial nanocellulose (preferably as defined herein),
bacterial cellulose/poly caprolactone nanocomposite film,
composite film of polyvinyl alcohol,
bifunctional linking cellulose nanocrystals, or
polylactide latex/nanofibrillated cellulose bio-nanocomposite.

Preferably, the 3D printer in step (2) is an ink jet printer, a sinter printer, a printer with melt layering.

As discussed above, the present invention provides nanocellulose composite chip obtained by said method.

Further Description of Preferred Embodiments

Our invention provides bacterial nanocellulose composite materials which contain DNA or RNA or modified nucleotides or further components for information processing. Moreover, our constructs (see detailed examples and explanations herein) as well as their broader principles allow the nanocellulose composite to become information processing (e.g. smart card, computer chip) as well as to become an intelligent material (e.g. to support wound healing).

In particular, the inventors have developed a nanocellulose composite comprising specific constructs and properties to work as a smart card/computer chip and/or to improve wound healing.

In particular, the present invention provides a bacterial nanocellulose composite, said bacterial nanocellulose comprising apart from the nanocellulose matrix DNA or RNA or modified nucleotides or further components for information processing.

The following versions are advantageous for all involved tasks:

Nanocellulose matrix (including suitable modified nanocellulose as well as modifying its surface)

with DNA or RNA or modified nucleotides and/or further components for information processing

(1) which is operated on by light-gated nucleotide-specific polymerase constructs or other nucleotide processing or nucleotide binding enzymes (e.g. Cid I polymerase, mu-polymerases, exonucleases, transcription factors, T4 polynulceotide kinase, adenyltransferase) including light-gated versions of these enzymes or fluorescent protein constructs (GFP, YFP, CFP protein fusions) including light-gated versions to achieve storage and information processing capabilities (smart card or computer chip). Nucleotides (DNA, RNA) are used as substrate and synthesized nucleotides for storage; i.e. the nucleotides represent the stored information (read-in, read-out);

OR

(2) with light-gated RNA polymerases, or protein translation system or enzymatic synthesis system or enzymes or sensors or light-gated versions of these enzymes to achieve molecular processing of information stored in nucleic acid or protein sequences; (“nano factory”);

OR

(3) pores (from proteins or nucleic acid) for electronic or optical properties or fluorescent proteins or transparent nanocellulose or nanocellulose with modifyable optical properties or organic polymers or graphene or fullerene or dyes or sensor proteins or enzymes (active on the surface, active expressed at the surface, hence actuators for “printing”) including typical refinement steps from classical computer chip technology to achieve interfacing with electronic components or representation of the results (output);

OR

(4) with sensor proteins and/or modified nanocellulose surface (including fluorescent proteins, monitoring proteins or light-gated versions of these or dyes) to monitor healing in wounds;

OR

(5) with growth factors, or kinases or receptors or enzymes or drugs or light-gated versions of these to reprogram wounds for optimal healing;

OR

(6) containing sensors or enzymes or pores, actuators or electronic parts to achieve an intelligent skin or tissue substitute.

The bacterial nanocellulose composite of the present invention can comprise one or more of each of the components (1) to (6) and combinations of the components (1) to (6), and optionally further component(s). The choice of the components will depend on the planned application of the bacterial nanocellulose composite, in particular molecular information processing.

These components are now further clarified:
(1) Nucleotide containing bacterial nanocellulose composite: Here the information processing and storage relies on nucleotides, for instance RNA or DNA. For the latter, three ground breaking publications (Church et al., 2012; Goldman et al., 2013, Grass et al., 2015) showed its unique capabilities to store information such as pictures, text or music. In particular by using DNA the information can be stored with Exabyte density (Church et al., 2012), be successfully retrieved with low error using error codes (Goldman et al., 2013) and stored with virtual unlimited life time (Grass et al., 2015). However, this required until now large machines. As a new step individual molecules, such as light-gated polymerases can function as polymerases (see e.g. German patent application No. DE 10 2013 004 584.3). However, this still required laboratory settings to retrieve the information successfully (see e.g. German patent application No. DE 10 2013 004 584.3) or vitrification of the DNA (Grass et al., 2015) to successfully preserve the DNA.
   The nanocellulose composite of the present invention now provides and brings all required information processing molecules and the nucleotide storage together, in an easy and efficient way and for very long time without any further steps.
(2) Light-gated RNA polymerases or protein translation system: The same considerations apply, however, here the information is stored and processed using RNA or protein sequences.
(3) The nanocellulose composite provides a natural surface. Modifying the surface by pores or modification of the nanocellulose itself yields electronic properties or provides optical properties. However, the said nanocellulose composite for information processing can now use these optical and electronical properties for displaying the stored information (e.g. by fluorescence) or for interfacing electronically or optically with other electronic devices (e.g. smart phone, computer, glass-fibre cable).
(4) Sensor molecules: The bacterial nanocellulose composite of the present invention can use sensor molecules to monitor things, for instance temperature in a wound. Again the nanocellulose composite protects the sensor molecules and also this renders the nanocellulose into an intelligent material for information processing.
(5) The nanocellulose composite can also interface with living objects using for instance growth factors to reprogram cells. Again the composite protects the components used for this interfacing.
(6) The same applies to encased cells for tissue transplants in the nanocellulose composite.

Further explanations of the individual components:

Ad (1), (2) Said nucleotide processing (in (1)) or protein processing (in (2)) molecules are preferably light-gated processing molecules. This means they are fused to a light-sensitive protein domain such as the BLUF domain or LOV domain or a cryptochrome domain so that their information processing activity can be switched on or off by light according to the specific wave length sensed by the light-gating domain.

Ad (4) A “sensor molecule” as used herein refers to a molecule or compound that senses a signal, such as light, temperature, ions, ligands, electric current, and responds to the signal or processes the signal via a conformational change, an (enzymatic) reaction or translocation. Also this sensing can be switched ON or OFF by fusion to a light-gating domain.

Ad (1), (2) and (4) Preferably, these light-gating domain(s) (Conrad et al., 2014) comprise or are BLUF domain, LOV domain or a cryptochrome, as described above.

Ad (1) Preferably, the protein(s) for nucleotide-based information processing are comprised from
polymerase(s) and exonuclease(s);
   such as DNA polymerase(s), RNA polymerase(s),
   e.g. T4 polynucleotide kinase
   Cid1 polymerase
   PolyU polymerase
   μ DNA polymerase

terminal deoxyncleotidyl (TdT) polymerase,

Ad (3) Preferably, the nanocellulose composite with a surface for electronic or optical properties to interface to electronic components or achieve output the modified surface (layer) is derived from:
ion channel(s) or pore(s);
   such as a GABA channel or other pores and ion channels (Buga et al., 2012)
membrane protein(s);
   lipoproteins, glycoproteins,
   such as bacteriorhodopsin,
receptors, for instance TNF receptors (Fricke et al., 2014)
or fluorescent proteins
or transparent nanocellulose or
or nanocellulose with modifyable optical properties
or organic polymers
or graphene
or fullerene
or dyes
or sensor proteins or enzymes (active on the surface, active expressed at the surface, hence
actuators for “printing”; a good example are two component systems composed of sensors and actuators/responders as known from various bacteria; described in Krüger et al., 2012)
or combinations thereof.

In some embodiments, domain(s) of the above mentioned protein(s) are used, such as catalytic or enzymatically active domains and/or binding domains.

Ad (1) Examples of fusion protein constructs of BLUF domains with polymerases or domains of polymerases are disclosed in German patent application of one of the inventors, DE 10 2013 004 584.3, which is enclosed herewith in its entirety.

Such examples are for instance:
fusion of a BLUF domain and T4 polynucleotide kinase,
fusion of a BLUF domain and PolyU polymerase (with a histidine in the active site),
fusion of a BLUF domain and PolyU polymerase (with an asparagine in the active site).

Ad (1, 2, 3, 4, 5): In one embodiment, the protein(s)/protein domain(s) comprising light-inducible or light-responding sensor domain(s) further comprise linker(s) and/or secretion signal(s) or signal peptide domain(s). This e.g. allows for the protein(s) or protein domain(s) to locate to/to be transported, or the like, to certain positions within the fibres of the nano cellulose (composite).

For example: planned application as chip card or smart card (DNA storage medium; i, ii, iii): Suitable proteins for nucleotide processing (i) are DNA polymerase(s) and RNA polymerase(s), such as Cid1 polymerase, PolyU polymerase, μ DNA polymerase, terminal deoxyncleotidyl (TdT) polymerase, or active domains thereof. Rapid readout is achieved by exonucleases, in particular with nucleotide specificity. Access of specific DNA strand-regions is achieved by DNA binding proteins, for example transcription factor binding proteins. Activity of any of these proteins can easily be monitored by fusing these proteins to a fluorescent protein domain e.g. GFP, YFP, CFP.

For controlling the activity of any of these proteins, light-gated protein domains are fused to these proteins. Resulting suitable light-gated information processing molecule(s) are thus:
fusion of a BLUF domain and T4 polynucleotide kinase,
fusion of a BLUF domain and PolyU polymerase (with a histidine in the active site),
fusion of a BLUF domain and PolyU polymerase (with an asparagine in the active site instead of the histidine, transforming the polymerase into a polyA polymerase).

Similarly, the protein sequence processing molecules (ii) as well as the nanocellulose surface properties (iii), e.g. pore proteins on the surface, can be controlled by light-gating them by fusion to a BLUF or other light-sensing domain and each can be monitored by fusion to a monitoring fluorescent domain. Again the nanocellulose composite is a huge advantage for compactly keeping and integrating all involved molecules together.

For the application as an intelligent nanocellulose composite for medical applications (intelligent plaster; iv, v, vi) suitable sensor molecule(s) embodied in the intelligent nanocellulose composite monitor the state of the wound, e.g. measure temperature, pH, inflammation (cytokinines) and show by a change in fluorescence the resulting state. Furthermore, the healing process should be improved by suitable programming the tissue or cells. For this the nanocellulose composite can contain growth promoting molecules such as growth factors (VEGF, EGF, PDGF), kinases, but also connective tissue stimulating components such as collagens. All these different components are well controlled, monitored and only selectively released in the nanocellulose composite including a suitable surface treatment of the nanocellulose (iii).

Ad (3) Actuator Molecules

The bacterial nanocellulose composite of the present invention comprises at least one information processing molecule in any of the embodiments (1 to 6). To deliver the output of the stored information by protein expression, by color change, or change of the nanocellulose surface properties in general, actuator molecules are used. The embodiment (3) is particularly suitable for getting a strong and easy readable output signal from the intelligent nanocellulose composite.

Said actuator molecules are preferably fluorescent molecule(s).

In a preferred embodiment, said actuator molecules are
(d) fluorescent protein(s),
(e) protein(s) or protein domain(s) comprising fluorescent domain(s), or (f) fusions of protein(s) or protein domain(s) with fluorescent protein(s) or fluorescent domain(s),

In one embodiment (see e.g. FIG. 11, FIG. 15), the actuator molecule(s) are selected from fluorescent protein(s) or protein(s) comprising fluorescent domain(s) or fusion protein(s) with fluorescent protein(s) or domain(s) and comprise GFP, CFP, YFP.

Strong colours for achieving a clear output signal from the nanocellulose composite are also Gaussia proteins and other fluorescent proteins.

Further possibilities include modifying the surface of the nanocellulose itself (in particular its transparency), insertion of pores (for interfacing with electronics and electronic read-out). The nanocellulose composite allows as an alternative also sandwich assays, use of dyes, of organic polymers or of graphenes to achieve a good output signal and interfacing ability with electronic components.

Ad (6) Cells

The bacterial nanocellulose composite of the present invention can comprise cells. This embodiment is particularly suitable for medical uses.

The basic form of the nanocellulose composite is here an intelligent plaster monitoring healing disturbance (pH change) by color change. Cells, however, turn the nanocellulose composite into a scaffold with cells for optimal integration into tissues. This in itself strongly augments the positive effects of the nanocellulose plaster. Furthermore, this can be exploited to more directly intensify the healing and regeneration process. Examples for cells to be used in the nanocellulose composite for this application are skin cells, stem cells (such as mesenchymal stem cells).

For example, the bacterial nanocellulose composite can comprise mesenchymal stem cells when it is to be used in wound healing.

For example, the bacterial nanocellulose composite can comprise specific tissue cells when it is to be used in tissue engineering (for instance it can use artificial lung tissue cells; see e.g. Stratmann et al., 2014)

The choice of the information processing molecule(s) and proteins in the nanocellulose composite will depend on the planned application of the bacterial nanocellulose composite.

Ad (7) Further components

The bacterial nanocellulose composite of the present invention can comprise further component(s).

These are preferably added if they can enhance the information processing capabilities of the composite either directly (smart card, computer chip) or the positive reprogramming of human body cells in medical applications.

In one embodiment, the nucleotide processing molecule(s) (1) and/or RNA/protein processing molecules (2) or surface modifying and output mediating actuator molecule(s) (3), sensor molecules (4), cellular reprogramming molecules (5) and/or cell(s) (6) and further component(s), if present, are embedded or encapsulated in the bacterial nanocellulose composite.

In this embodiment, the component(s) can be added to the bacterial nanocellulose.

The information processing molecule(s) (1) to (5) and/or further component(s) (7), if present, can also be co-produced during the bacterial fermentation or bacterial expression of the bacterial nanocellulose itself. Thereby, particular expression constructs and cell biological cell lines are utilized. This was tested and is most easily achieved for said molecules by expression from one construct or expression from several plasmids in one bacterial strain such as E. coli high expression strains.

In one embodiment, the information processing molecule(s) (1) to (6) and/or further component(s) (7), if present, are covalently attached to the nanocellulose,
such as via linker, anchor groups or nanocellulose surface attachment after pH activation and/or crosslinking by UV activation.

### DESCRIPTION OF FURTHER PREFERRED EMBODIMENTS

This application claims priority of German patent applications DE 10 2015 005 307.8 and DE 10 2015 005 308.6 filed Apr. 27, 2015, the contents of which are hereby incorporated in their entirety by reference.

Embodiment as Smart Card or Storage Chip or Computing Chip

We start from an already established highly efficient genetic process for nanocellulose generation (Kralisch et al., 2015). Said process uses gram negative aerobic bacteria, for instance Komagataeibacter (earlier name: Acetobacter or Gluconacetobacter). Growth medium: Hestrin-Schramm medium made from water, glucose, yeast extract plus pepton, pH buffering—wherein numerous alternative media, for instance from plants are known. Furthermore we want to emphasize that also other bacteria can be used, in particular cyano bacteria, as described by the Brown group, University of Texas (Nobles and Brown, 2008). Transfer of the nanocellulose synthesis into cyano bacteria strongly enhances the yield. Nanocellulose is generated in a bioreactor at moderate temperatures (25-30° C.) at the surface of the liquid culture (interface to air) as a structure stable hydro-polymer (solid phase fraction about 1%, hydrogel). The polymer is harvested at the surface. From a molecular perspective, nanocellulose is generated between cell wall and external membrane of the bacterial cell by a cellulose synthase complex which produces nanocellulose as a quite long glucose chain molecule from UDP-glucose monomers. The glucose polymers leave the cell as cellulose elementary fibrils through pores at the surface and aggregate to microfibrils. This self-assembly together with cell division and branching resulting therefrom, leads to the characteristic three dimensional fiber network. In natural conditions the fiber network serves for protection against drying-out, enemies, lack of oxygen or nutrients as well as UV-radiation. These properties complement optimal other tissue implants (e.g. chondrofillerliquid).

Subsequently the nanocellulose is populated with sensors and actuators (selected proteins, which prepare the matrix for utilization as a chip; FIG. 1). For this it is only necessary to express both this typical molecular biology constructs as well as the nanocellulose and add to the proteins a suitable secretion sequence so that they find their optimal place in the fiber network.

An innovative smart card or even chip card is generated: A nanocellulose foil is armed with biological switches (proteins).
a. Further embodiment as chip card and intelligent foil for technical applications:

The following components are used for the improvement of chip cards from nanocellulose with organic switches:
light-gated protein domains (these are able to receive an external light signal which change the directly coupled, active acting (actuator) protein such that the light signal is either stored or read-out again. To achieve this two-component systems are useful;
light-gated ion channels (Müller et al., 2015) to change the electronical properties of the chip card; as well as
modification of the storage chip (as shown above; suitable expression vectors and secretion sequences are of course used for this).

There are already efforts for an optical transparent “paper” for electronic displays (Kralisch et al., 2014). Nanocellulose is already used as LED display in computer components since some time (Ferguson et al., 2012). However, there the nanocellulose is only used as transparent cover.

The essential novelty of our invention arises by the combination of the imbedded components with the nanocellulose. Thus there is the combination of a light-gated polymerase with nanocellulose. By this arises a chip card in which important substrates such as cofactors and nucleotides can be used in the chip card for many cycles, in particular for data storage with the help of the light gated polymerase (DPA 10 2013 004 584.3). Advantageous is also the combination of nanocellulose with biological storage molecules, in particular bacterial rhodopsines (Imhof et al., 2014; Yao et al., 2005; Barnhardt et al., 2004) to use the chip card like this for data storage. FIG. 1 shows the intelligent chip card made from nanocellulose (cross-grid in the back) with embedded molecular switches (current and signal modulating pores, switches (cylinders) or proteins, with high resistance or condensator properties (open squares).

b. Further Improvements
a) Further vector constructs for signal processing properties (including light-gated ion channel sequences, membrane proteins, required lipid sequences)
b) Expression systems for production: We use in our invention and according to the state of the art not only E. coli but also Acetobacter, which is already known from nanocellulose production as well as further advantageous systems for generation of nanocellulose known to the expert (e.g. blue algae).
c) Include engineering methods from semi-conductor industry: Irradiation and photoresist, doping, imprinting, spiking-up, integration of further molecular components
d) Usage as tissue implant and achieved improvements by this, in particular better Monitoring of healing in the tissue as well as improved tissue healing and signal transmission properties of nanocellulose.
e) Usage as “intelligent material” and achieved technical applications
f) Usage as detector/“intelligent dust” (for instance in comparison to imprinting-based detection instruments etc)
g) Usage of “intelligent nanocellulose” as printer, storage- and computing device

c. Embodiment as Intelligent Material for Broader Applications

Starting from the intelligent nanocellulose or nanocellulose foil it is possible to complement or modify the matrix polymer, in particular by usage of
a) organic polymers
b) polynitrocellulose
c) silicon, with or without polysinalisation (classical chip)

For the main intended uses as tissue replacement or as smart card usually composites are produced (in particular according to “a)”, usage together with plastics as main component in chip cards, or according to “c)” usage of inert Silicon as tissue replacement). The further broader applications of the nanocellulose composite gain most of all from the advantages in the two main applications:

Intelligent Chip Card:

This can be the combination of nanocellulose with graphenes, or with organic polymers, including such which can serve as battery. Important is to state that we use nanocellulose hydrogel in the inside, since then the substrates etc. for the imbedded molecules described above are at hand. Starting from this, there is in particular the option to replace many components of metal nature (condensors, resistors, transistors) or from plastics with proteins or nanocellulose or polymers from a) to c) in this biologically transformed chip card.

Combined Embodiment:

Together, both approaches yield further synergies in the application of nanocellulose together with our specific embedded components, for instance for muscle stimulation, cardiac monitoring or similar medical applications or a competitor products to “electronic skin” (Tee et al., 2012, who, however, use instead of our above components metals, in particular nickel and self-healing plastics), in doing so, the skin transplants gets by these procedures much better sensor properties.

(1) Intelligent nanocellulose, in particular modified nanocellulose foil, obtained by including of specific signal processing molecules, cells or actuator molecules in the nanocellulose.

The intelligent nanocellulose is suitable as composites of cells and protein structures for the chip card technology.

(2) Intelligent nanocellulose of (1) characterized in that the nanocellulose is not only used as transparent material such as in the LED technique, but further more actively as chip card, since the nanocellulose obtains further advantageous information carrier features due to the embedded molecular-biological switches, namely specific sensor or actuator molecules, respectively.

(3) Use of the intelligent nanocellulose as “intelligent material”, in particular as detector/“intelligent dust” (such as in comparison to imprinting detection media etc).

(4) Use of the intelligent nanocellulose as printer, storage medium and processing medium.

(5) Use of the intelligent nanocellulose ecological/environmentally friendly computer chip or chip card with low content of plastics/synthetic materials and/or metals.

Embodiment Wound Healing

We start from an already established high efficient genetic process for nanocellulose generation (Kralisch et al., 2015). Said process uses gram negative aerobic bacteria, for instance Komagataeibacter (earlier name: Acetobacter or Gluconacetobacter). Growth medium: Hestrin-Schramm medium made from water, glucose, yeast extract plus pepton, pH buffering—wherein numerous alternative media, for instance from plants are known. Furthermore we want to emphasize that also other bacteria can be used, in particular cyano bakteria, as described by the Brown group, University of Texas (Nobles and Brown, 2008).

Transfer of the nanocellulose synthesis into cyano bacteria strongly enhances the yield. Nanocellulose is generated in a bioreactor at moderate temperatures (25-30° C.) at the surface of the liquid culture (interface to air) as a structure stable hydro-polymer (solid phase fraction about 1%, hydrogel). The polymer is harvested at the surface. From a molecular perspective, nanocellulose is generated between cell wall and external membrane of the bacterial cell by a cellulose synthase complex which produces nanocellulose as a quite long glucose chain molecule from UDP-glucose monomers. The glucose polymers leave the cell as cellulose elementary fibrils through pores at the surface and aggregate to microfibrils. This self-assembly together with cell division and branching resulting therefrom, leads to the characteristic three dimensional fiber network. In natural conditions the fiber network serves for protection against drying-out, enemies, lack of oxygen or nutrients as well as UV-radiation. These properties complement optimal other tissue implants (e.g. chondrofillerliquid).

Subsequently the nanocellulose is populated with sensors and actuators (selected proteins, which in particular show or support wound healing, respectively, or which prepare the matrix for utilization as a chip; FIGS. 1 and 2). For this it is only necessary to express both this typical molecular biology constructs as well as the nanocellulose and add to the proteins a suitable secretion sequence so that they find their optimal place in the fiber network.

By this we obtain a nanocellulose (e.g. as hydrogel) populated with wound-healing promoting molecules and cells as novel tissue replacement.

a. Improved Tissue Replacement and Wound Transplant or Wound Cover:

The following proteins are particular useful for the usage as sensor and hence as monitors for wound healing: proteins for measuring, in particular from two component systems (or also with an aptamer-component), which then measure metabolites, temperature, ion concentrations, tension-compression (important in the implant) as well as interactions; furthermore the measurement read-out is transmitted by fluorescence (GFP component) or by gene expression change (two component systems) or other signals. Fluorescent proteins or two component systems are simply imbedded in the hydrogel and they glow to show their state.

Natural growth factors are active agents for wound healing (VEGF, Erythropoetin, NGF, EGF etc.) and can be used in our composite. The same applies to collagen I, II, X, aggrekan, catabolic matrix degrading enzyme MMP-2 as well as human mesenchymal stem cells which provide support as well. In this application the hydrogel is kept liquid and absorbable, such that it is typically completely absorbed after some time (typically within several weeks). FIG. 2 shows the optimized tissue replacement from nanocellulose (cross-grid in the back) with integrated growth-promoting biomolecules (flashes) and mesenchymal stem cells (large shape).

Specifically tested were different collagenes (active molecules, actuators) as well as GFP-constructs (sensors) to test the intactness of a nanocellulose implant, however, as described above, there are many further possibilities, for instance the integration of further sugar molecules (tissue sugar code) to the nanocellulose (glycomic), to strongly promote wound healing.

b. Further Embodiments
a) Further vector constructs for signal processing properties (including light-gated ion channel sequences, membrane proteins, required lipid sequences)
b) Expression systems for production: We use in our invention and according to the state of the art not only E. coli but also Acetobacter, which is already known from nanocellulose production as well as further advantageous systems for generation of nanocellulose known to the expert (e.g. blue algae).
c) Use as tissue implant and achieved improvements by this, in particular better Monitoring of healing in the tissue as well as improved tissue healing and signal transmission properties of nanocellulose.

c. Further Embodiment as Intelligent Material for Broader Applications

Starting from an intelligent nanocellulose or nanocellulose foil it is possible to complement or modify the matrix polymer, in particular by usage of
a) organic polymers
b) polynitrocellulose
c) silicon, with or without polysinalisation (classical chip)

For the main intended use as tissue replacement such composites are produced (in particular according to “c)” usage of inert Silicon as tissue replacement). The further broader application of the nanocellulose composite gains most of all from the advantages in the two main applications:

Wound Healing:

Particularly advantageous is the integration of “b)” and “c)” as printed circuit, this renders the surface again more sensitive and suitable to support wound healing while preventing problems in the healing process. These additions are novel in the combination with nanocellulose as matrix and promise decisive improvements. Furthermore, wound healing is simultaneously supported and monitored by proteins or sensors if suitably supplied.

Combined Embodiment:

Together, both approaches yield further synergies in the application of nanocellulose together with our specific embedded components, for instance for muscle stimulation, cardiac monitoring or similar medical applications or a competitor products to “electronic skin” (Tee et al., 2012, who, however, use instead of our above components metals, in particular nickel and self-healing plastics), in doing so, the skin transplants gets by these procedures much better sensor properties.

(1) Intelligent nanocellulose, in particular modified nanocellulose foil, obtained by including of specific signal processing molecules, cells or actuator molecules in the nanocellulose.

The intelligent nanocellulose is suitable as composites of cells and protein structures for wound healing (such as band-aid, transplant), characterized in that
monitoring of wound healing can be improved, or
improving and stimulating wound healing by including the disclosed molecules and stem cells directly into the nanocellulose (as composite or covalently).

(2) Use of the intelligent nanocellulose as intelligent skin transplant and for general monitoring of the health state.

(3) Use of the intelligent nanocellulose for stimulus conduction or for improved healing and as neuro transplant and for muscle stimulation, including the heart.

The following examples and drawings illustrate the present invention without, however, limiting the same thereto.

### BRIEF DESCRIPTION OF THE DRAWINGS

FIG. 1. Bacterial nanocellulose composite for information processing: use in chip technology.

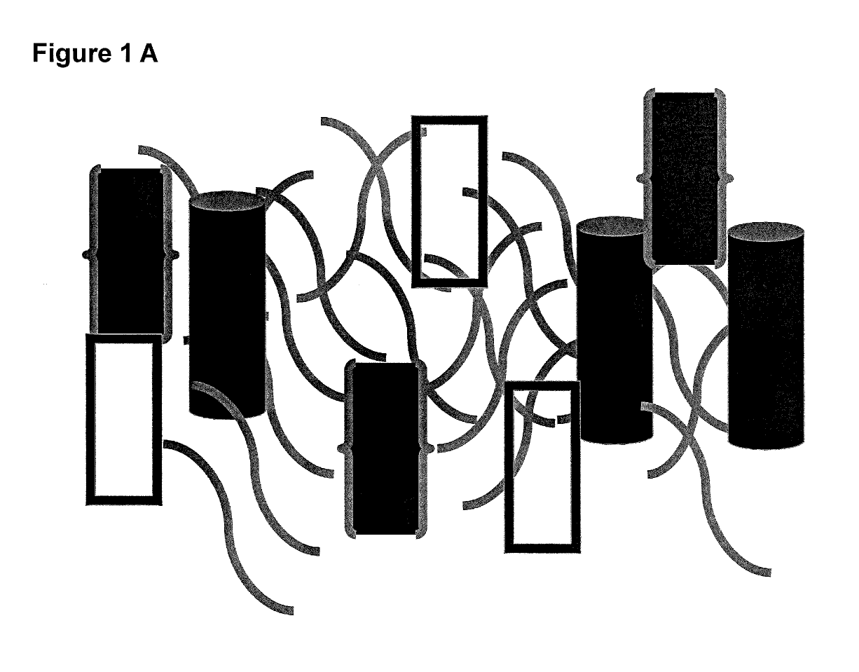

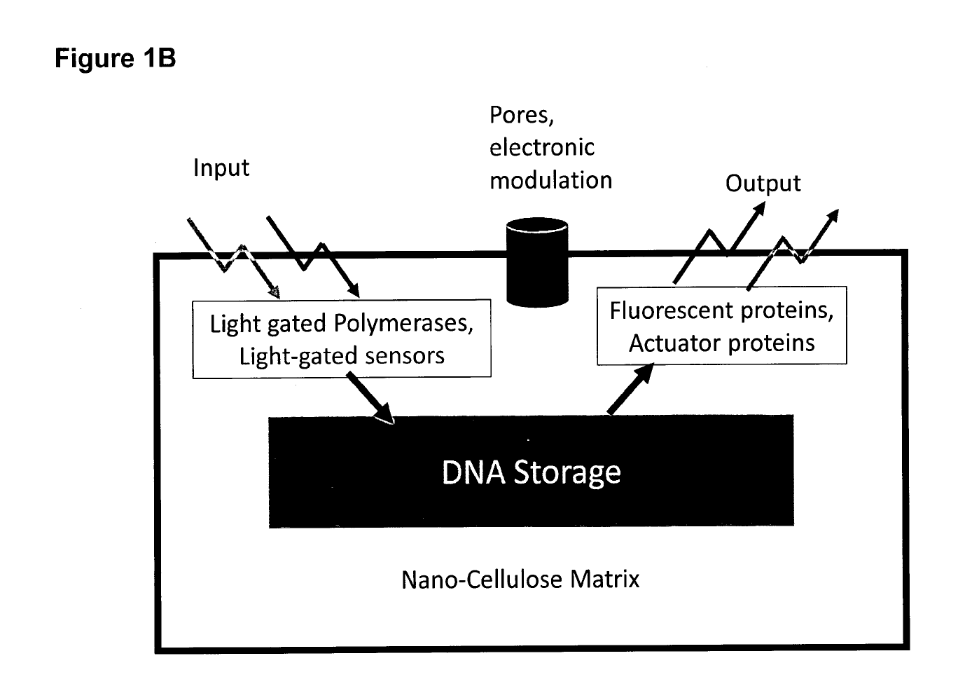

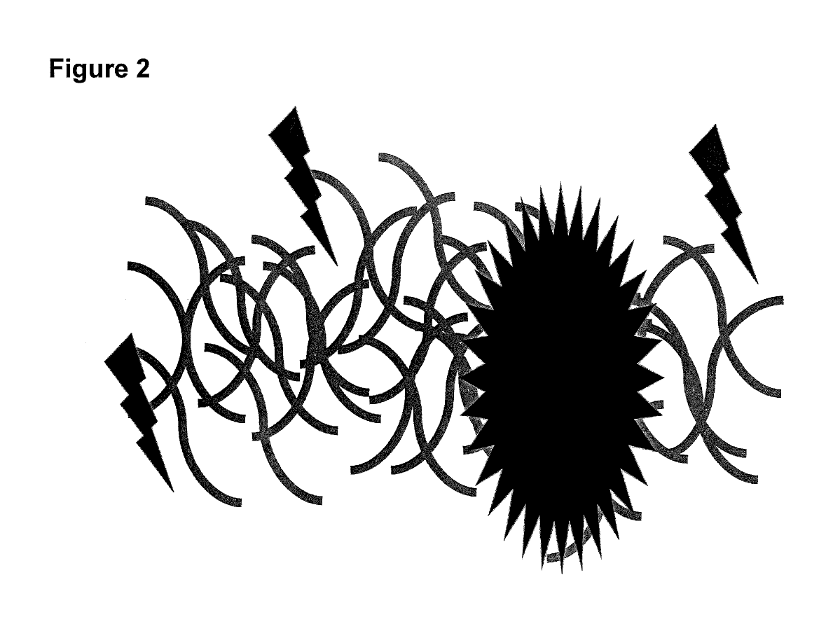

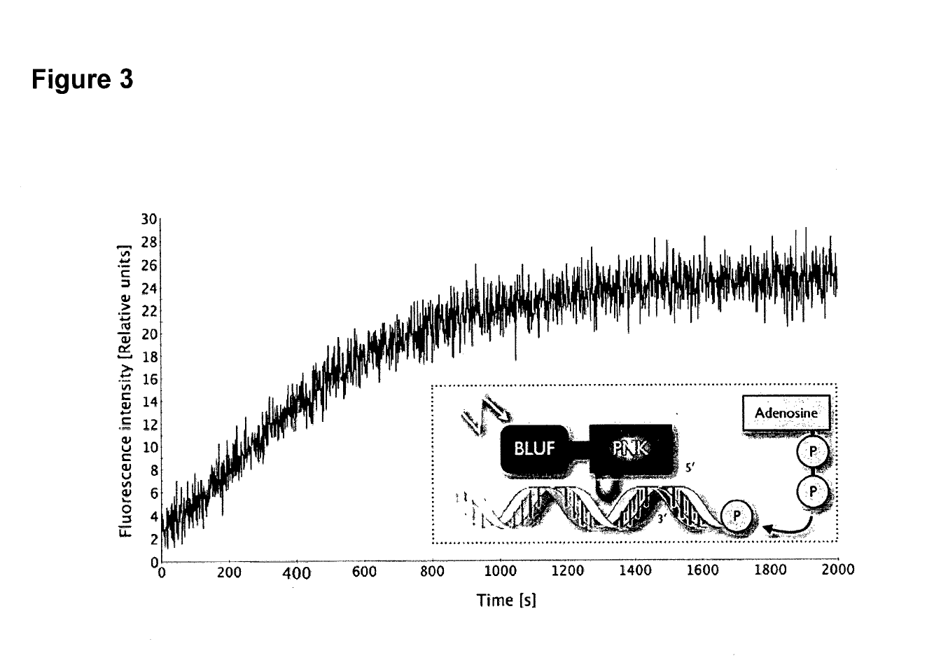

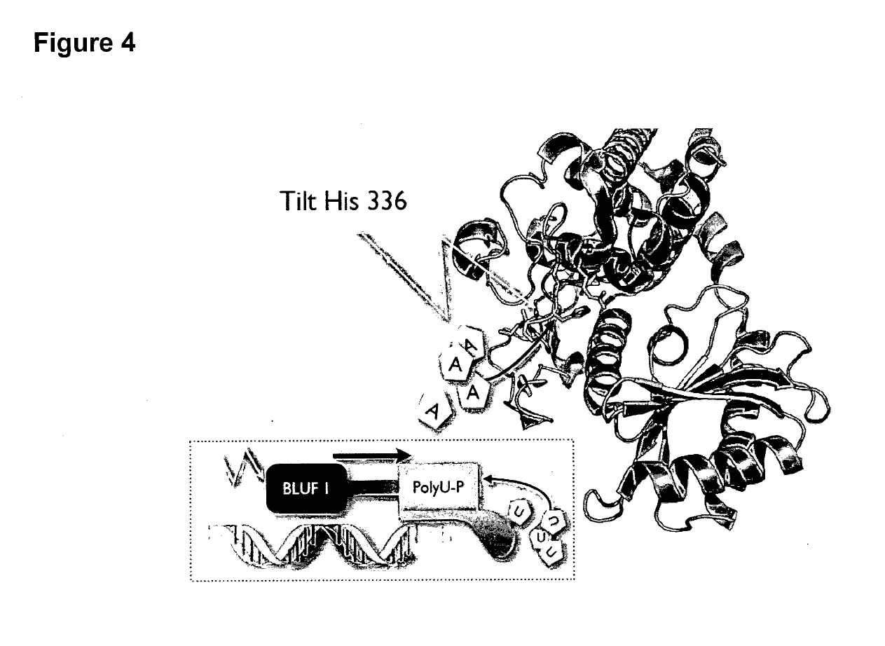

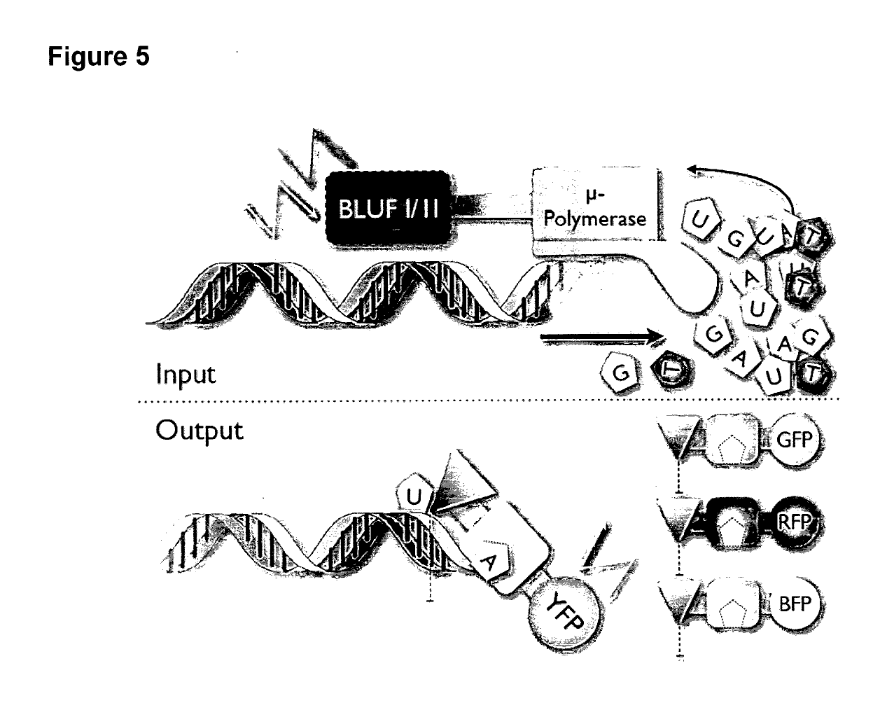

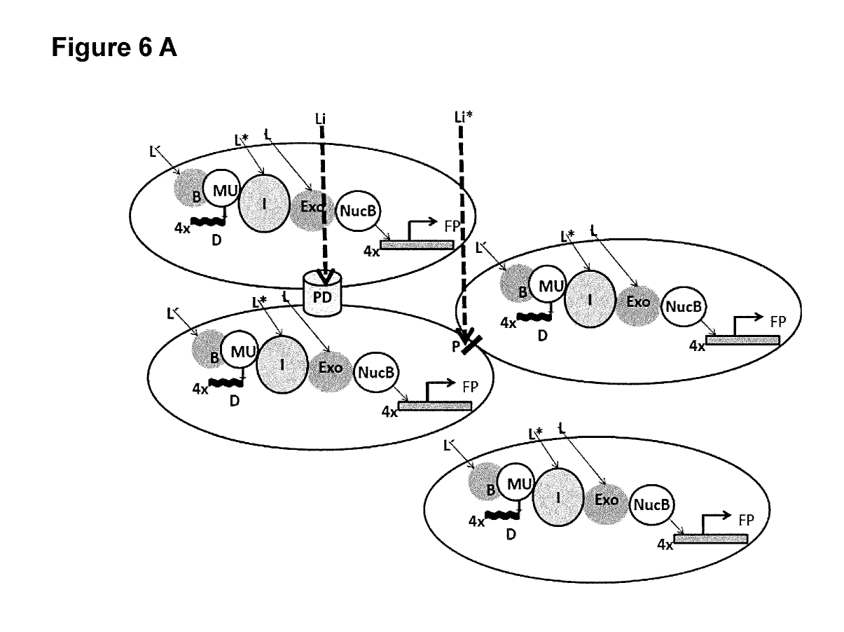

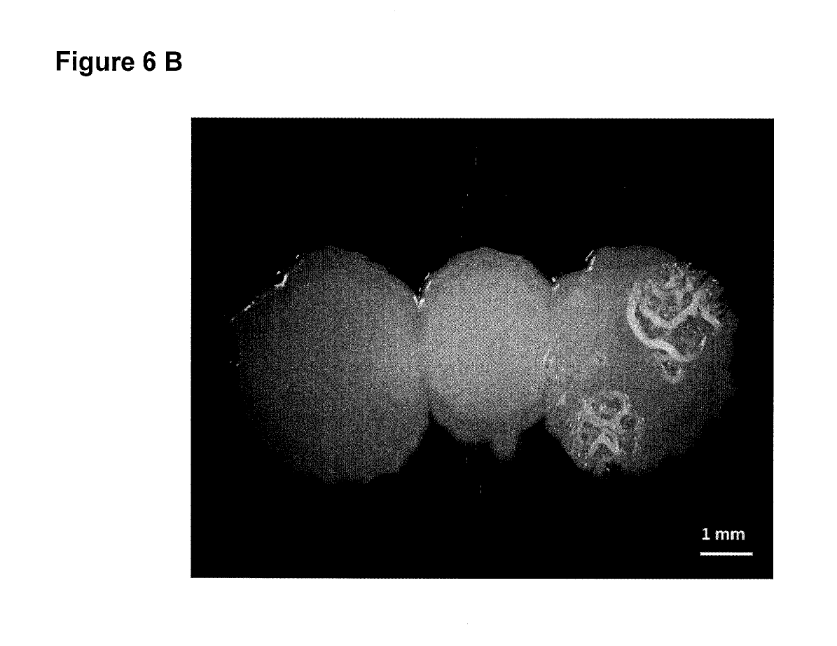

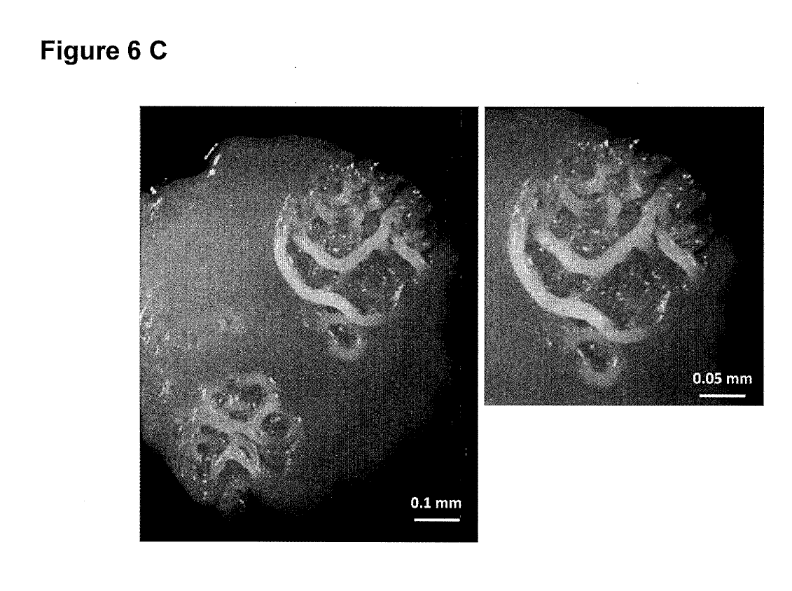

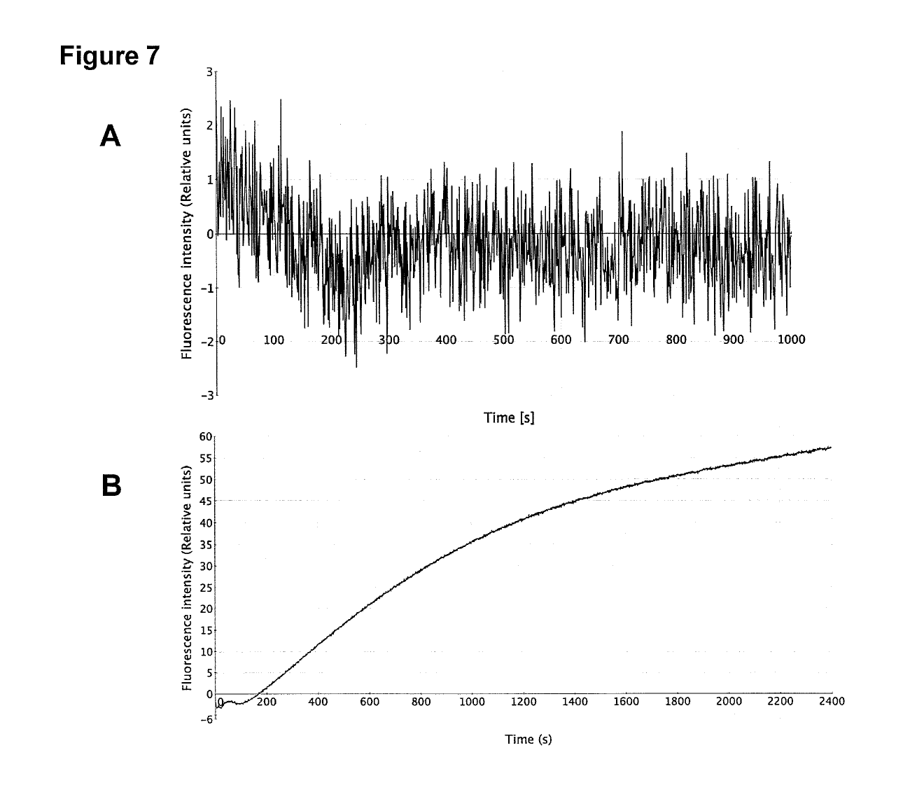

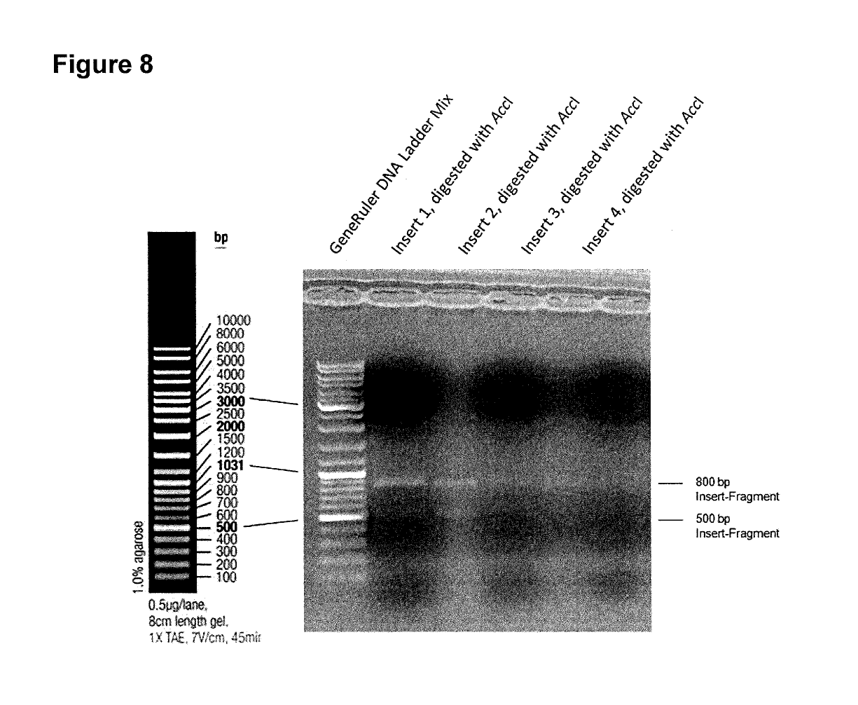

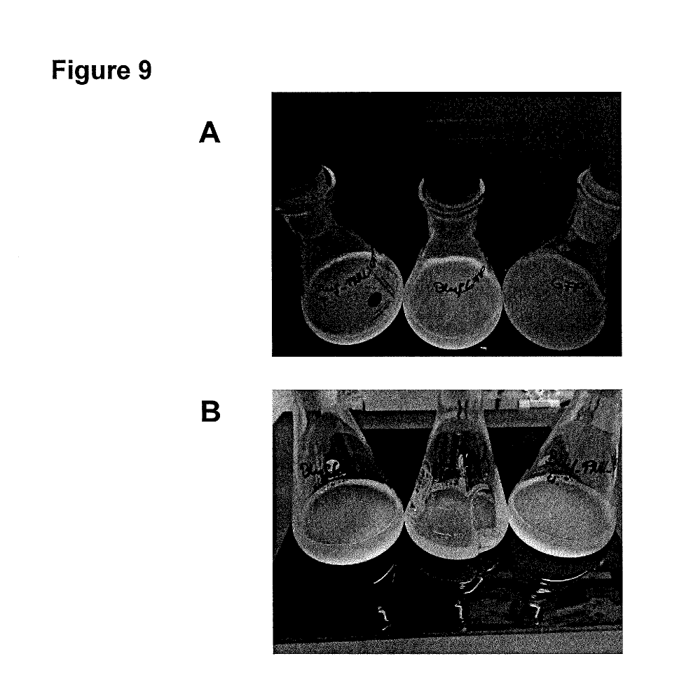

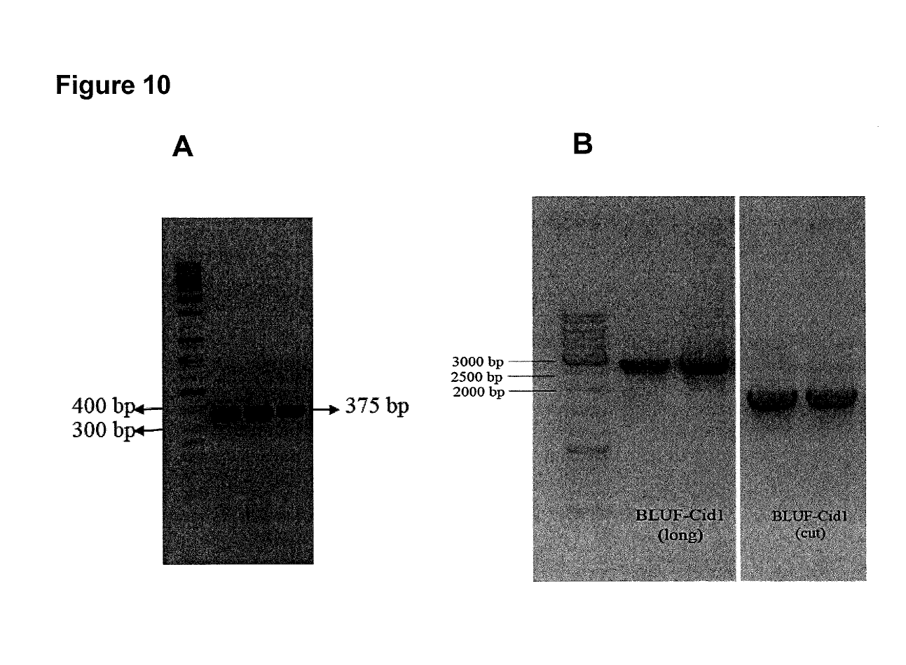

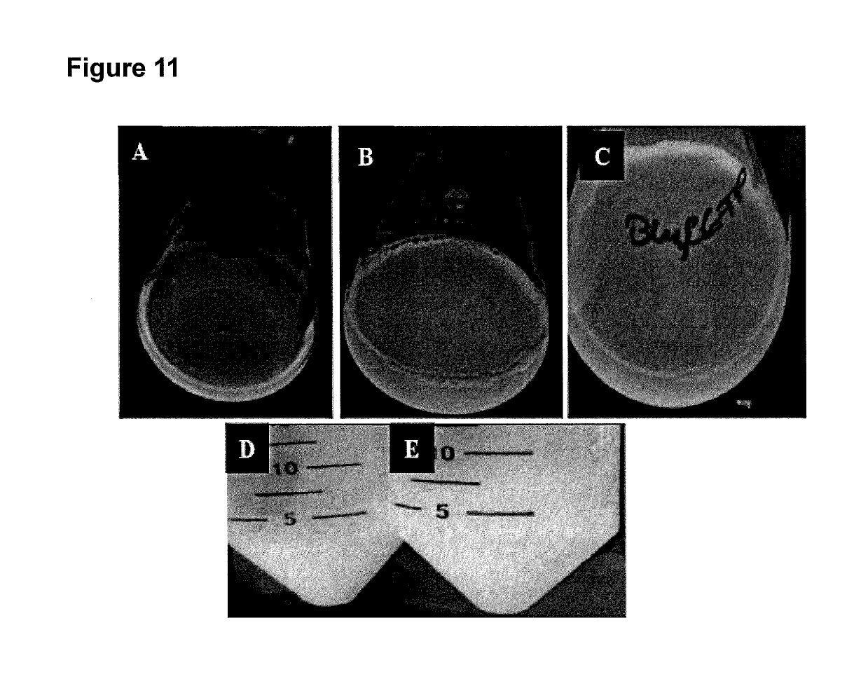

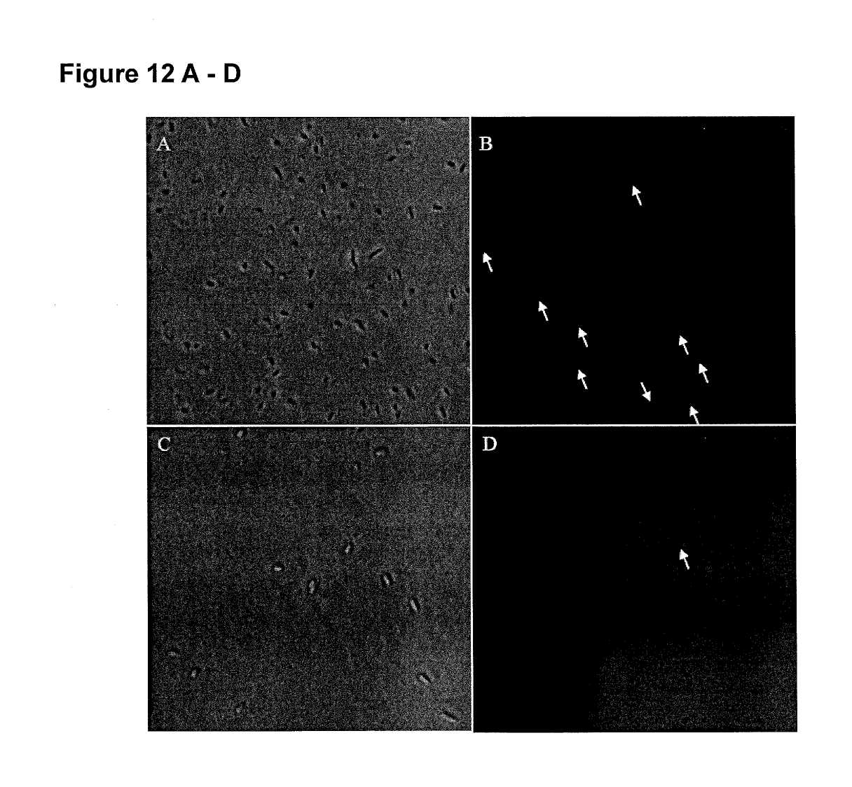

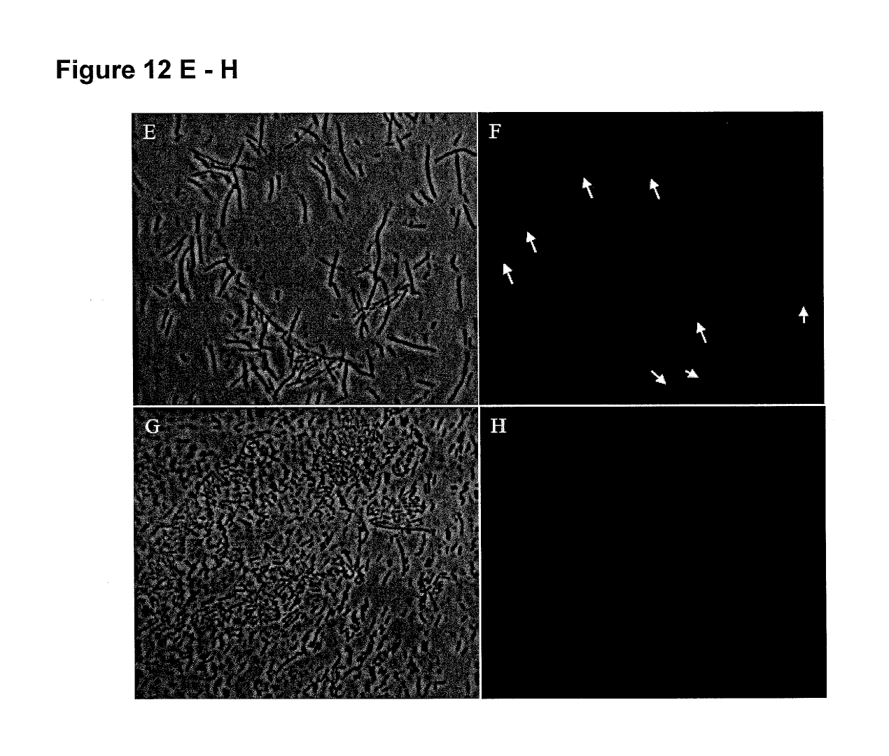

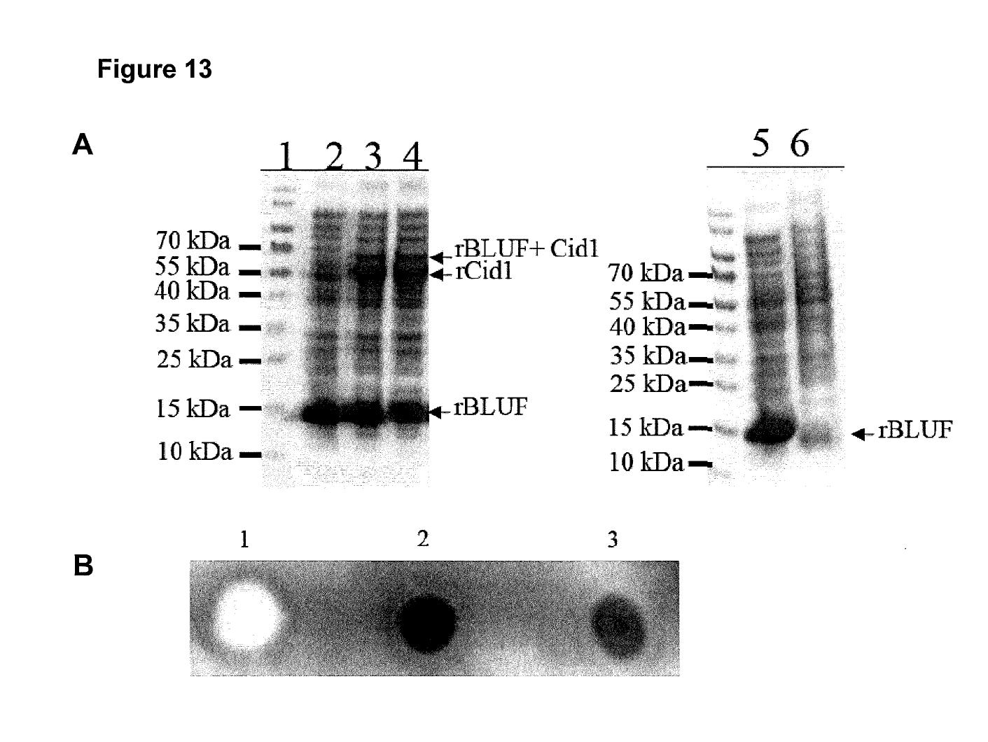

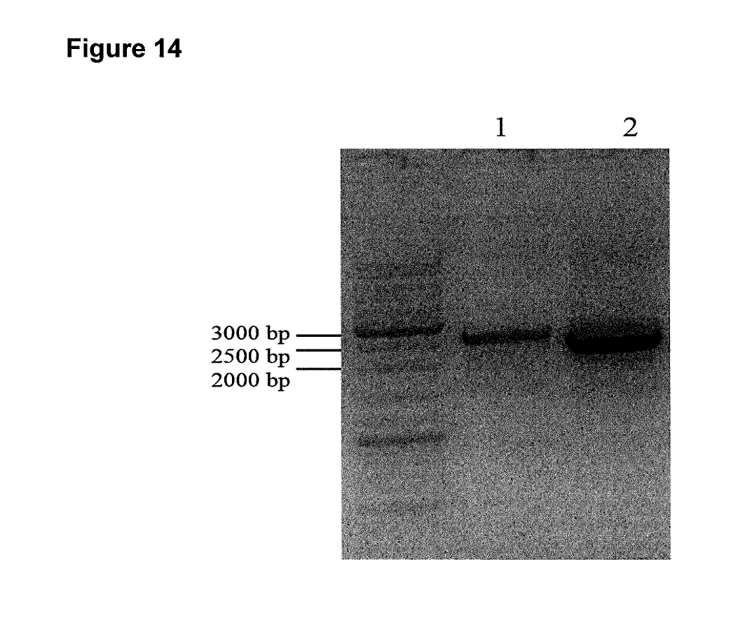

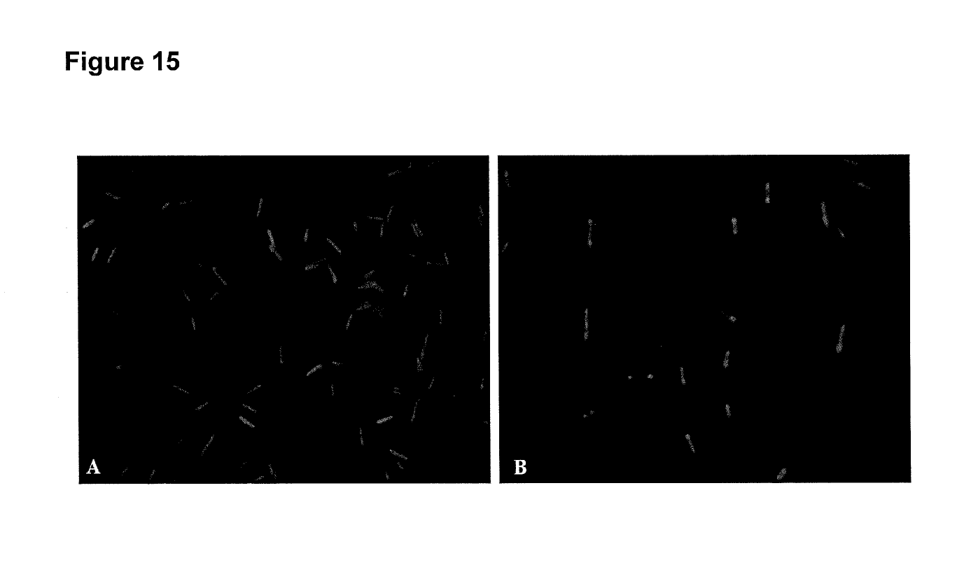

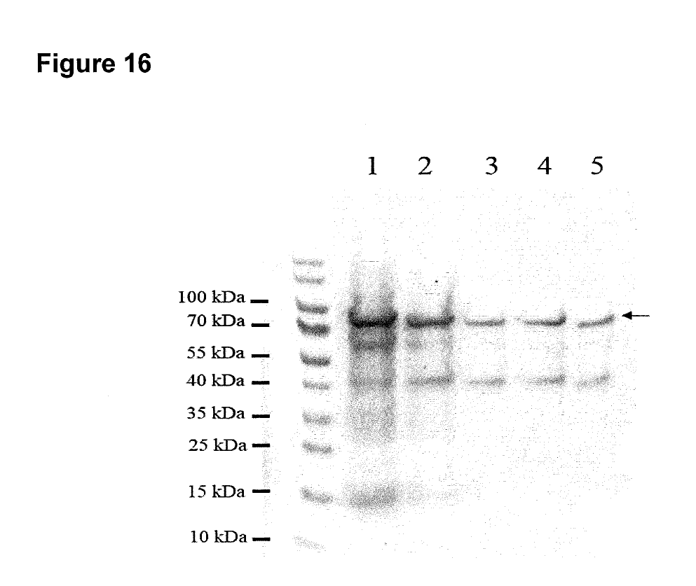

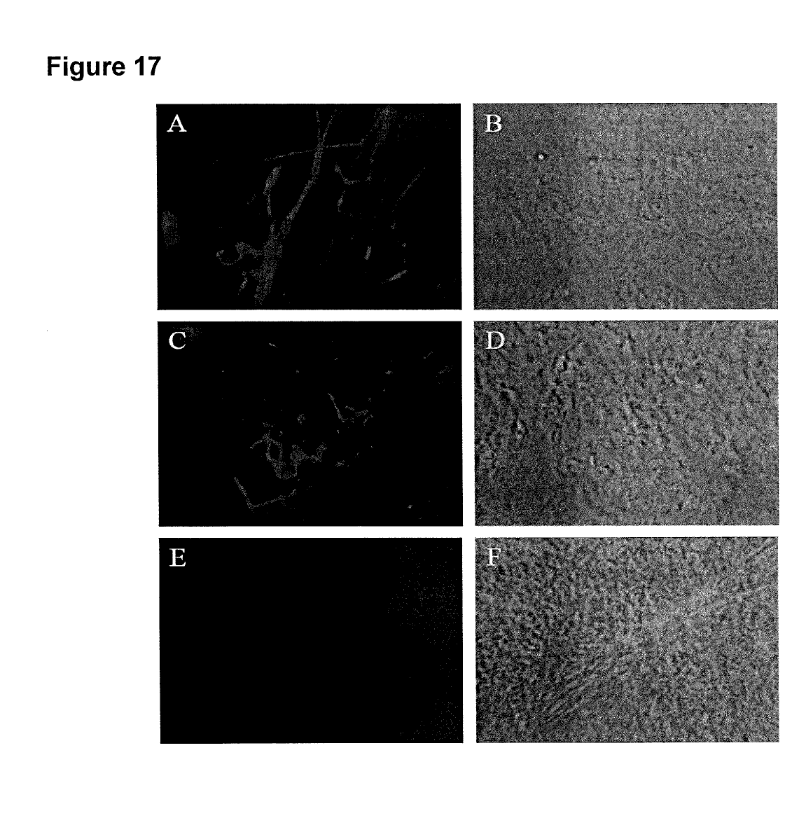

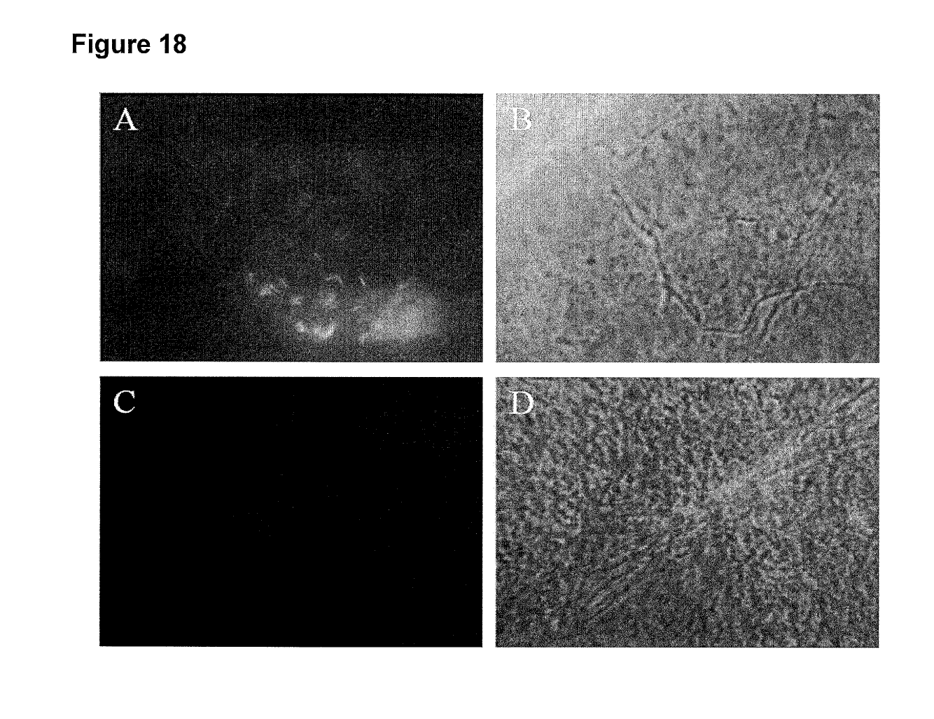

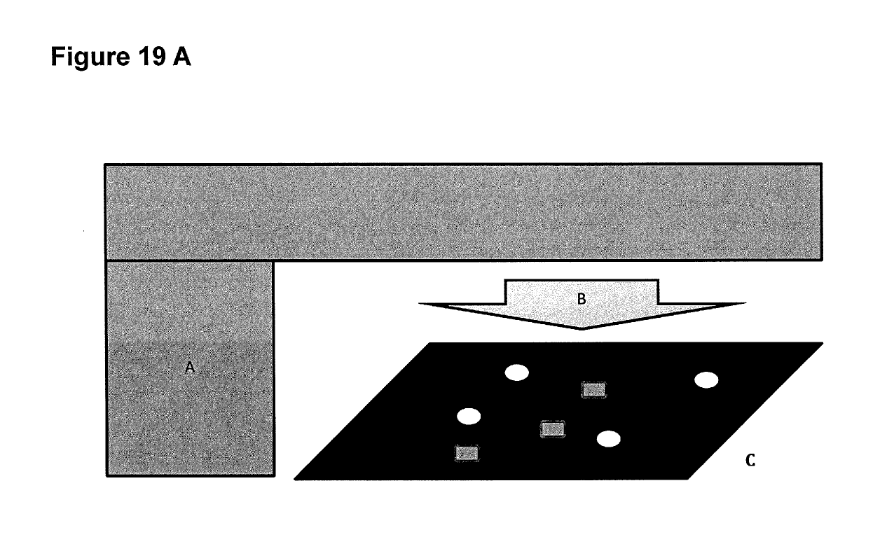

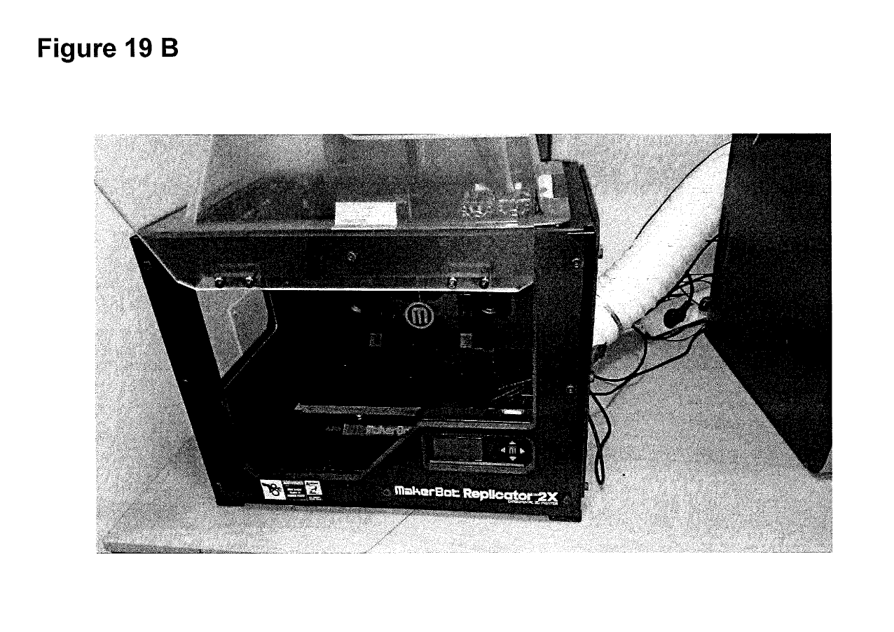

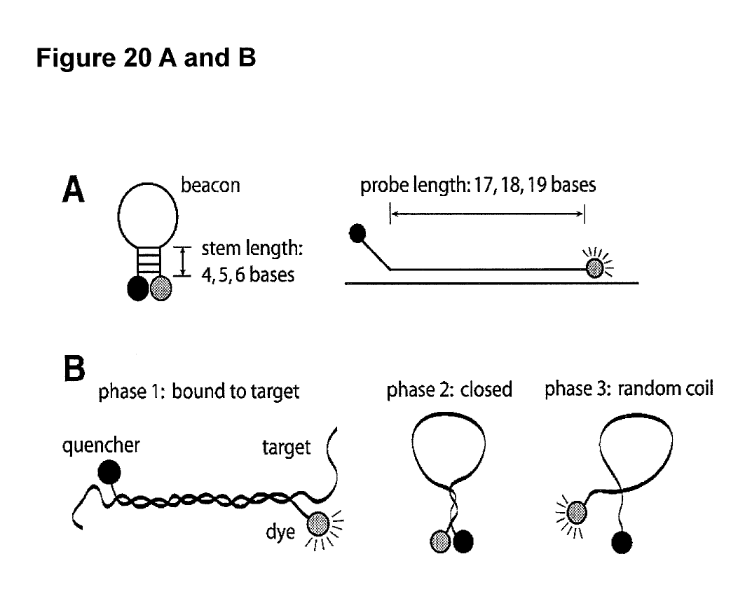

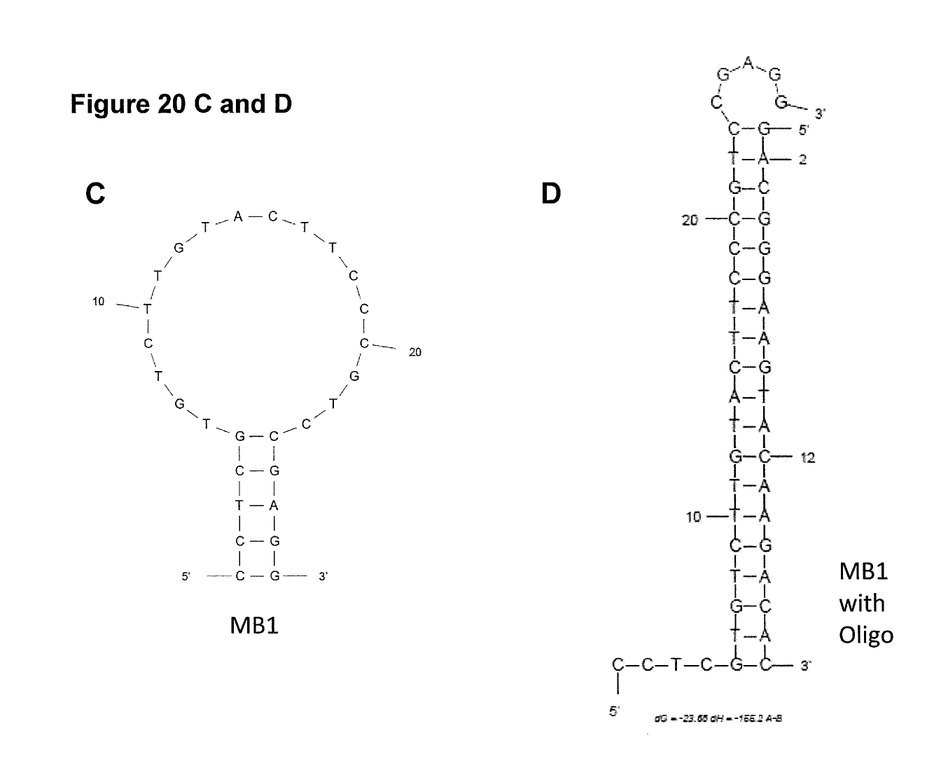

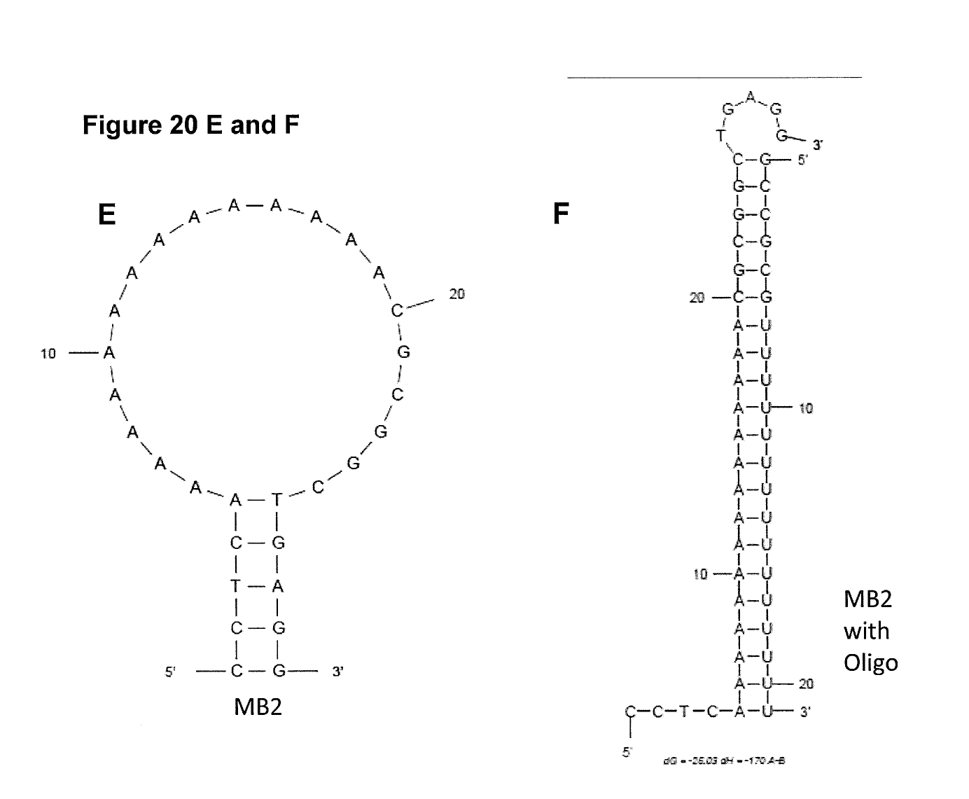

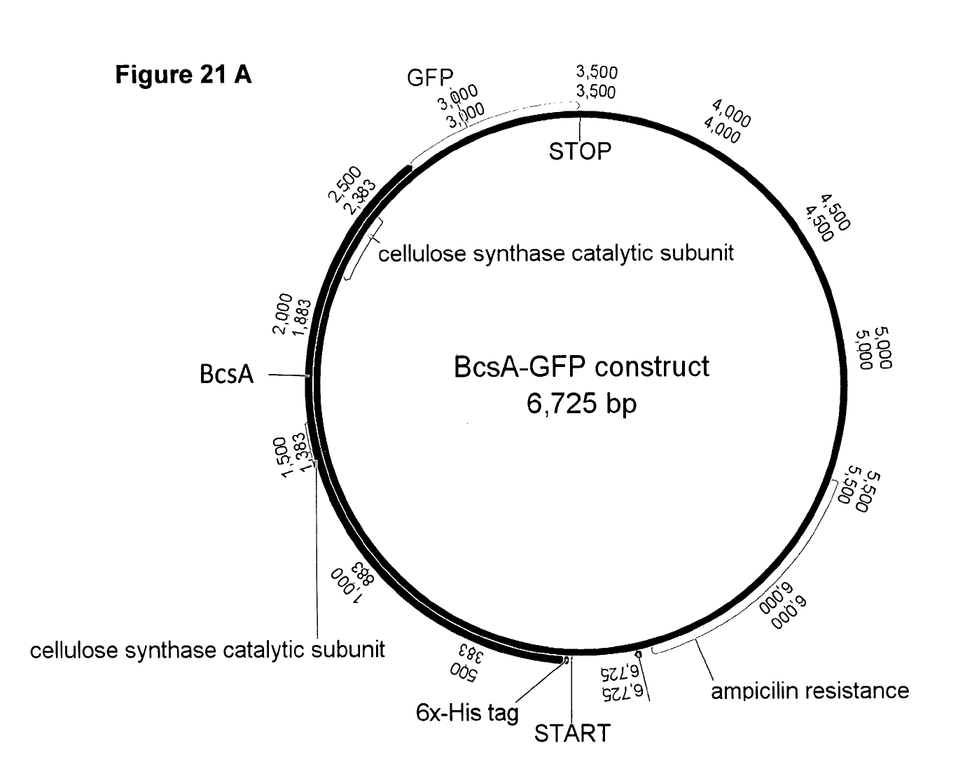

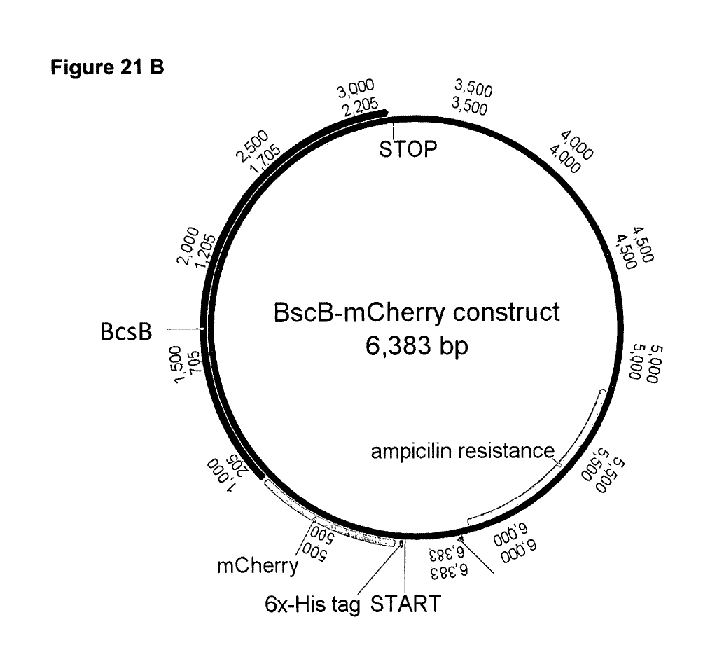

A, Key components: Shown is a chip card made of bacterial nanocellulose (shown as fibers in the background) with embedded molecular switches (current- and signal-modulating pores, switch molecules (cylinders) or proteins having high resistance or capacitor characteristics, respectively (open squares).

B, In action: Nanocellulose composite containing information processing molecules (DNA/RNA polymerases or protein processing molecules) which may be controlled in their activity by different light wave lengths (top) by fusion to a light-sensing domain. Output is mediated by fluorescent proteins, actuator proteins, again in different wave-length. Membrane pores and modulation of membrane properties (optical, electronical properties of the nanocellulose surface) allows modulation of electronic properties and interfacing to electronic devices.

FIG. 2. Bacterial nanocellulose composite for information processing: use in tissue engineering.

Shown is an optimized tissue implant of bacterial nanocellulose (shown as fibers in the background) with embedded growth-promoting biomolecules (arrows) and mesenchymal stem cells (star). Further molecules may include monitoring (GFP) and sensor molecules to monitor inflammation and temperature.

FIG. 3. Bacterial nanocellulose composite key components: Achieving light-gated DNA input and output—light-controlled phosphate transfer.

Measurement (top): assay for T4 kinase DNA elongation constructs using processed fluorescent oligonucleotides (Song and Zhao, 2009), for monitoring their activity; construct calculations to predict joined cooperative changes after Halabi et al. (2009) and Lee et al. (2008). The aim (bottom): construction of protein chimeras which transfer signals from the light harvesting BLUF domain to the effector domain, here polynucleotide kinase (PNK), to achieve on or off switching of effector activity.

FIG. 4. Bacterial nanocellulose composite key components: Achieving light-gated DNA input and output—light directed PolyU polymerase.

Top: A histidine in the PolyU polymerase domain (PDB file shown: 4FH3) determines A or, in alternative position, U elongation (Lunde et al., 2012). The histidine 336 may be tilted by light to achieve rapid changes in substrate specificity according to user-specified sequences of As and Us. Bottom: Activity of the PolyU polymerase has again to be under light-control by fusion to a BLUF domain.

FIG. 5. Bacterial nanocellulose composite key components: Achieving light-gated DNA input and output—active DNA storage design. Input (top): μ-DNA polymerase is used to achieve light-gated (light-specific BLUF domain/μ-DNA polymerase constructs for each nucleotide) and template free DNA synthesis.

Output (bottom): light-gated exonuclease constructs (triangles) are fused to specific nucleotide-binding domains (squares) and trigger different fluorescent proteins for readout.

FIG. 6. Active DNA storage in bacterial nanocellulose composite.

Previous efforts used living bacteria in a biofilm to achieve this storage (see DPA 10 2013 004 584.3). However, this can be difficult to manage, to maintain, to control—in particular, bacterial cells divide, need nutrients and escape by mutations control. The bacterial nanocellulose composite of the present invention solves all these problems and leads to a much more reliable, improved storage.

A, artificial biofilm blueprint for active multicomponent DNA storage: Each nanocellulose composite carries light-gated constructs for active DNA storage; input: light gated (L′) BLUF domain B controls MU DNA polymerase constructs, four such constructs (4×) write GATC nucleotides into DNA (D); regulatory light (L*) gated interface domain I; output: light-gated (L) exonuclease (Exo) together with nucleotide binding domain (NucB) directs fluorescent protein (FP) expression or signalling, again four different constructs are required. Furthermore, nanocellulose composite interconnections have to be modified by light-gated (Li, stippled arrows) opening of pores (for DNA PD or ion current P) to achieve controlled multi-cellular DNA storage and exchange as well as to achieve circuits with electronic properties.

B, Comparison: engineered patterns in a real biofilm: We show the high self-repair potential, the patterning of the biofilm, and restoration of biofilm formation potential. Readout is done here by different optical appearance; available are also different FP constructs and lacZ constructs. In the example (B. subtilis bacteria) key sensor histidine kinase genes were artificially deleted (kinC, kinD). This abolishes biofilm formation or any tight connections (see FIG. 6A) between cells (left colonies: no biofilm formed). There are spontaneous mutations in the strong biofilm repressor sin R which turn tight interaction back on and achieve patterning of colonies with biofilm forming and non-forming regions (right colony). Change in DNA content for all these specific mutations is actively monitored and visible. For large-scale active DNA storage it is highly advantageous to introduce the light gated and monitoring constructs in one or several nanocellulose composites (including various technical improvements compared to FIG. 6A described in other sections of this document).

C, close up looks on the engineered biofilm (scales are indicated, focus: patterned region).

FIG. 7. Key components of nanocellulose composite: comparing active T4 kinase readout to control condition.

A, Control base-line level.

B, Active T4 kinase readout.

FIG. 8. Nanocellulose composite imbedded molecular components: BLUF domain.

Shown is testing of PCR fragments and vector constructs. 800 bp Fragment of the BLUF construct, testing the AccI cut, which should and does cut ⅓ of the fragment.

FIG. 9. Nanocellulose composite imbedded molecular components: Monitoring light gated control of enzyme function by GFP constructs.

A, Comparing BLUF-PNK-GFP, BLUF-GFP, GFP construct, Fluorescence in the dark. All three show fluorescence, the additional BLUF-domain enhances fluorescence.

B, Comparing BLUF-GFP, control and BLUF-PNK-GFP construct. UV plus daylight shows that the BLUF-GFP constructs respond with fluorescence under daylight.

FIG. 10. Nanocellulose composite imbedded molecular components: Creating light-gated nucleotide processing enzymes (demonstrated here for Cid1, a polyU RNA polymerase).

A, Verification of BLUF-coding sequence from the transfected bacteria (Rosetta strain) by PCR reaction.

B, Verification of BLUF-Cid1 (long) and BLUF-Cid1 (cut) from the transfected bacteria (M15 strain) by PCR reaction.

FIG. 11. Nanocellulose composite imbedded molecular components: Light-gated control of fluorescence.

Results of a BLUF-GFP construct. No blue light leads to inactive BLUF domain and hence far less fluorescence.

Shown are cultured bacteria in Lysogeny broth under UV.

A, negative control, only non-transfected E. coli in LB media,

B, positive control, induced E. coli with GFP cultured in 20 ml of media,

C, induced E. coli with BLUF-GFP construct in 20 ml of media,

D, lysate of non-induced E. coli (negative control),

E, lysate of E. coli with BLUF-GFP construct.

FIG. 12. Nanocellulose composite imbedded molecular components: Light-gated GFP monitoring construct is demonstrated.

Here we show light-gated (blue light mediate) control of GFP fluorescence.

The comparative study of different GFP expression in the BLUF-GFP construct under different conditions (magnification 100×).

A, 16 hrs of cultivation in daylight (phase contrast),

B, 16 hrs of cultivation in daylight (under UV),

C, 16 hrs of cultivation in dark (phase contrast),

D, 16 hrs of cultivation in dark (under UV),

E, 24 hrs of cultivation in daylight (phase contrast),

F, 24 hrs of cultivation in daylight (under UV),

G, 24 hrs of cultivation in dark (phase contrast),

H, 24 hrs cultivation in dark (under UV).

FIG. 13. Nanocellulose composite imbedded molecular components: Light-gated RNA polymerase CidI.

A, Shown is SDS-PAGE with protein lysates of recombinant BLUF and BLUF-Cid1 constructs.

Lane 1—marker, line 2—BLUF-GFP, lane 3—BLUF-Cid1 (cut), lane 4—BLUF-Cid1 (long), lane 5—BLUF-GFP, lane 6—negative control, lysate from the non-induced cells.

B, Western-blot analysis of different BLUF constructs. Spot A—BLUF-GFP, Spot 2—BLUF-Cid1 (cut), spot 3—BLUF-Cid1 (long).

FIG. 14. Nanocellulose composite: Nanocellulose generation.

Amplification of BcsA and BcsB. Line 1—BcsA, line 2—BcsB.

FIG. 15. Nanocellulose composite: Nanocellulose together with green or red reporters.

Visualization of E. coli transformed by BcsA/BcsB with fluorescent reporters.

A, BcsA protein fused with GFP,

B, BcsB protein fused with mCHERRY.

FIG. 16. Nanocellulose composite: Nanocellulose production.

Here time-dependent expression of rBcsB.

Lane 1—whole cell lysate of BcsB, lane 2-6 hrs induction, lane 3-21 hrs induction, lane 4-30 hrs induction, lane 5-45 hrs induction. Arrow depicts the BcsB protein.

FIG. 17. Nanocellulose composite:

Nanocellulose stained by mCHERRY protein.

A and C, stained nanocellulose under red fluorescence.

B and D, the corresponding picture with phase contrast.

E and F, negative control, only nanocellulose (non-stained) under UV (E) with corresponding phase contrast (F).

FIG. 18. Nanocellulose composite:

Nanocellulose stained by GFP protein.

A, stained nanocellulose under green fluorescence.

B, corresponding picture with phase contrast.

C and D, negative control, only nanocellulose (non-stained) under UV (C) with corresponding phase contrast (D).

FIG. 19. Nanocellulose composite:

Flexible 3D Printer used in the 3D printing experiments for the nanocellulose composite.

FIG. 20. Detection of polymerase activity of light-gated RNA polymerase CidI with a molecular beacon assay.

A, shows design and structural parameters of molecular beacons.

B, shows that molecular beacons in solution can have three phases: bound to target, closed and random coil.

C and D, show the structure of the beacon MB1 alone (C) and with the oligonucleotide (D), so the beacon in the target bound state; also measured when the Klenow polymerase is active).

E and F, show the structure of the beacon MB2 alone and with the oligonucleotide (so beacon in the target bound state; also measured when the CidI polymerase is active).

FIG. 21. Plasmid constructs for bacterial expression of nanocellulose.

A, Ligated BcsA into the pQU-30-mCHERRY-GFP vector. After the digestion with BamHI and SalI, the mCHERRY-coding region was excised and replaced with BcsA-coding region.

B, Ligated BcsB into the pQU-30-mCHERRY-GFP vector. After the digestion with KpnI and BlpI, the GFP-coding region was excised and replaced with BcsB-coding region.

### EXAMPLES

### Example 1 Light-Gated Polymerases and Kinases and their Application for Active DNA Storage in Nanocellulose Composite

Light-gated proteins provide not only an important basis for neurogenetics, they are also very useful to achieve storage, recall and modification of nucleotide sequences for long-term information storage as DNA. We test here BLUF- and LOV-domain fusion constructs fused to Cid I polymerase and T4 polynucleotide kinase. Fusion constructs are established and validated for their sequence. The light-gating property is tested in fluorescence assays regarding nucleotide extension as well as by GFP expression regarding processivity. In conclusion, these constructs allow light-gated elongation of nucleotide sequences, either by phosphorylation or by polyuridylation. We describe further constructs and modifications and that the full functionality of an active DNA storage can be obtained.

1. Materials and Methods

1.1 Structural and Statistical Predictions

Calculation of engineered mutations were performed using the SCA MATLAB toolbox published by Ranganathan et al. (see Halabi et al, 2009).

All tested primer constructs for different BLUF domains, specifically:

Construct series A—BLUF(cut)-Linker-Cid I

Construct series B—Cid I(A)-Linker-BLUF(cut)-Linker-Cid I(B)

Construct series C—Cid I-Linker-BLUF

In each series, different linkers were tested (see below).

1.2. Molecular Cloning and Tests

For cloning, the pGEM®-T easy Vector system (Promega Corp.) was used.

2. Results

2.1. Key Steps for Active DNA Storage

A direct connection from molecular processing in cells and DNA to technical computers is necessary to achieve speed and calculation potential. Electronic properties of DNA (Timper et al. 2012) are difficult to handle We disclose herein for linking DNA information processing to in silico processing step-by-step in an efficient way to light-gated proteins (Liu et al., 2012). Light-gated proteins allow (i) control of their own and other enzyme activities, (ii) gene expression and protein-protein interactions, as well as (iii) to achieve patterning and directing cell to cell communication and integration of circuits. Containment features control the high biological repair and replication potential of such biobricks (Shetty et al., 2008) which together achieve extremely robust active DNA storage technology without negative side-effects or uncontrolled risks.

FIGS. 3-6 demonstrate which critical steps need to be achieved and a blueprint of the design with experimental data: Light gated enzyme elongates or modifies DNA according to a signal (FIG. 3), Light gated polymerase synthesizes a new sequence according to light-signal (FIG. 4; inset: Crystal structure of RNA Poly U polymerase and the critical histidine which directs A or U incorporation, and could be tilted by light input). Light gated constructs achieving light-directed DNA synthesis and DNA-sequence readout via optical signals (FIG. 5). FIG. 6A sketches active DNA storage applying the constructs shown in FIG. 3-5 in a bacterial biofilm. FIGS. 6 B and C show self-repair potential and experimental results for an own engineered biofilm.

2.2. Testing Principles of DNA Storage

See FIGS. 6A to C.

3.3. Demonstrating Active DNA Storage Enzymes

In the following, the key steps for active DNA storage were all examined in detail. Different ways to achieve active, light gated nucleotide synthesis were compared (T4 Polynucleotide kinase and Cid I poly U polymerase) as well as different light-gated domains to control their activity (BLUF domain or LOV domain). Furthermore, monitoring of construct activity was either done indirectly (activity monitoring by fluorescent oligo in vitro after protein purification) or directly (activity monitoring of the construct within the bacteria by GFP construct). Furthermore, the resulting product is either tested biochemically (modification of the oligo), optically (fluorescence and modification by blue light) or by sequencing of the product.

We summarize in the following all different combinations tested and the evidence collected for the construct activities:

BLUF-T4 Polynucleotide kinase construct: Truncated BLUF domain with optimal length according to SCA analysis is fused to polynucleotide kinase. The PCR product was cloned in plasmids, expressed and verified and the protein purified. For details, see below.

Furthermore, control experiments measured T4 kinase activity using fluorescent oligos compared to negative controls.

b) Three different BLUF-Cid I constructs test control of Cid I polymerase activity by BLUF.

c) As an alternative, different LOV constructs test Cid I activity. These constructs perform similarly well, however, the required wave length for light-gating Cid I is different.

d) Direct monitoring within bacteria by GFP constructs. Constructs include: GFP alone, BLUF-GFP, BLUF-Cid I-GFP, BLUF-Cid I-BLUF-GFP.

Fluorescence is observed for the constructs and the BLUF domain controls Cid I as well as GFP activity.

The accession numbers for these different proteins and genes are as follows:
Polynucleotide kinase gene (NC_000866 REGION: complement (134002 . . . 134907) from complete T4 genome) with polynucleotide kinase protein (accession number KJ477686.1) from enterobacteria phage T4.
   See SEQ ID NO. 20 (showing the 301 amino acid sequence of polynucleotide kinase of enterobacteria phage T4).
Poly(A) polymerase Cid1 (accession number NP_594901) gene from Schizosaccharomyces pombe.
   See SEQ ID NO. 9 for the 405 amino acid sequence.
The BLUF domain was obtained as part of the YcgF gene and protein (Tschwori et al., 2009; Tschwori et al., 2012). The used DNA for the BLUF domain was hence gene ycgF (accession number AAC74247.3) from the E. coli strain DH5-α, amplicon from 1-375 nt (125 AA). See SEQ ID NOs. 1-7, wherein
   SEQ ID NO. 1 shows the 403 amino acid sequence of BLUF E. coli;
   SEQ ID NO. 2 shows aa 1-84 of SEQ ID NO. 1 and SEQ ID NO. 3 the respective nucleotide sequence;
   SEQ ID NO. 4 shows aa 1-144 of SEQ ID NO. 1 and SEQ ID NO. 5 the respective nucleotide sequence;
   SEQ ID NO. 6 shows aa 1-125 of SEQ ID NO. 1 and SEQ ID NO. 7 the respective nucleotide sequence.

The used DNA for the Cid1 construct started with poly(A) polymerase Cid1 (accession number NP_594901) from the yeast Schizosaccharomyces pombe [972h-].
See SEQ ID NO. 9 for the 405 amino acid sequence.
SEQ ID NO. 10 shows aa 33-405 of SEQ ID NO. 9 and SEQ ID NO. 11 the respective nucleotide sequence;
SEQ ID NO. 12 shows aa 1-377 of SEQ ID NO. 9 and SEQ ID NO. 13 the respective nucleotide sequence;
SEQ ID NO. 14 shows aa 1-331 of SEQ ID NO. 9 and SEQ ID NO. 15 the respective nucleotide sequence;
SEQ ID NO. 16 shows aa 332-405 of SEQ ID NO. 9 and SEQ ID NO. 17 the respective nucleotide sequence.

Different constructs used these proteins but modified the DNA encoding these to achieve an optimal construct for our purposes. In particular, Cid1 polymerase synthesizes poly U stretches, but can be modified to synthesize poly A (Lunde et al., 2012) and our novel constructs allow to switch the CidI activity on and off by having blue light exposure there or not.

As the sequences have been modified, the resulting nucleotide sequences are shown in the following.

2.4 Polynucleotide Kinase (PKN)

The following construct was established: BLUF domain (Blue light responsive protein domain) is optimized in its length (so that it transmits cooperative changes) to T4 polynucleotide kinase. Such construct was compared to control conditions in a fluorescence monitoring assay of T4 polynucleotide kinase. FIG. 7 compares active T4 kinase readout to control condition. FIG. 11B shows that PKN-GFP or GFP alone can be controlled by blue light/day light using the BLUF domain.

2.5. BLUF/Cid I Construct

The first construct attaches the predicted active part of a BLUF signalling protein (amino acids 1-84 of SEQ ID NO. 1) to a complete Cid I polymerase protein (amino acids 33-405 of SEQ ID NO. 9). The Cid I part is located at the C-terminal part of the designed fusion protein.

Construct A—BLUF(cut)-Linker-Cid I

```
BLUF
SEQ ID NOs. 2 and 3
MLTTLIYRSHIRDDEPVKKIEEMVSIANRRNMQSDVTGILLFNGSHFFQLLEGPEEQVKMIYRAICQDPRHYNIV
ELLCDYAPA
                                Apa I Hind III
AAAAAA.GCGCGCGC.GGGCCC.AAGCTT.
ATGCTTACCACCCTTATT
ATGCTTACCACCCTTATTTATCGTAGCCATATACGTGACGACGAACCTGTCAAAAAAATCGAAGAAATGGTTTCG
ATAGCAAATCGCAGGAACATGCAGTCTGACGTAACAGGGATCTTACTGTTTAATGGTTCTCATTTTTTCCAGCTT
CTGGAAGGTCCGGAAGAACAGGTTAAAATGATATATCGGGCTATATGCCAGGATCCACGGCACTATAATATTGTT
GAGCTGCTGTGCGATTACGCGCCTGCT
                  TGCGATTACGCGCCTGCTGGTGGTGGTGGA
                  TCCACCACCACCAGCAGGCGCGTAATCGCA
Linker
SEQ ID NOs. 18 and 19
GGGGS GGGGS GGGGS
TACGCGCCTGCT
GGTGGTGGTGGAAGCGGCGGCGGCGGCAGC
GGTGGTGGTGGAAGCGGCGGCGGCGGCAGCGGCGGCGGAGGGAGC
                              GGCGGCGGCGGCAGCGGCGGCGGAGGGAGCAGCTACCAAAAG
                              CTTTTGGTAGCTGCTCCCTCCGCCGCCGCTGCCGCCGCCGCC
Cid I
SEQ ID NOs. 10 and 11
SYQKVPNSHKEFTKFCYEVYNEIKISDKEFKEKRAALDTLRLCLKRISPDAELVAFGSLESGLALKNSDMDLCVL
MDSRVQSDTIALQFYEELIAEGFEGKFLQRARIPIIKLTSDTKNGFGASFQCDIGFNNRLAIHNTLLLSSYTKLD
ARLKPMVLLVKHWAKRKQINSPYFGTLSSYGYVLMVLYYLIHVIKPPVFPNLLLSPLKQEKIVDGFDVGFDDKLE
DIPPSQNYSSLGSLLHGFFRFYAYKFEPREKVVTFRRPDGYLTKQEKGWTSATEHTGSADQIIKDRYILAIEDPF
EISHNVGRTVSSSGLYRIRGEFMAASRLLNSRSYPIPYDSLFEEAPIPPRRQKKTDEQSNKKLLNETDGDNSE*S
TOP
GGCGGAGGGAGC
AGCTACCAAAAGGTCCCT
AGCTACCAAAAGGTCCCTAATTCGCACAAGGAATTTACGAAGTTTTGCTATGAAGTGTATAATGAGATTAAAATT
AGTGACAAAGAGTTTAAAGAAAAGAGAGCGGCATTAGATACACTTCGGCTATGCCTTAAACGAATATCCCCTGAT
GCTGAATTGGTAGCCTTTGGAAGTTTGGAATCTGGTTTAGCACTTAAAAATTCGGATATGGATTTGTGCGTGCTT
ATGGATTCGCGCGTCCAAAGTGATACAATTGCGCTCCAATTCTATGAAGAGCTTATAGCTGAAGGATTTGAAGGA
AAATTTTTACAAAGGGCAAGAATTCCCATTATCAAATTAACATCTGATACGAAAAATGGATTTGGGGCTTCGTTT
CAATGTGATATTGGATTTAACAATCGTCTAGCTATTCATAATACGCTTTTACTTTCTTCATATACAAAATTAGAT
GCTCGCCTAAAACCCATGGTCCTTCTTGTTAAGCATTGGGCCAAACGGAAGCAAATCAACTCTCCTTACTTTGGA
ACTCTTTCCAGTTATGGTTACGTCCTAATGGTTCTTTACTATCTGATTCACGTTATCAAGCCTCCCGTCTTTCCT
AATTTACTGTTGTCACCTTTGAAACAAGAAAAGATAGTTGATGGATTTGACGTTGGTTTTGACGATAAACTGGAA
GATATCCCTCCTTCCCAAAATTATAGCTCATTGGGAAGTTTACTTCATGGCTTTTTTAGATTTTATGCTTATAAG
TTCGAGCCACGGGAAAAGGTAGTAACTTTTCGTAGACCAGACGGTTACCTCACAAAGCAAGAGAAAGGATGGACT
TCAGCTACTGAACACACTGGATCGGCTGATCAAATTATAAAAGACAGGTATATTCTTGCGATTGAAGATCCTTTC
GAGATTTCACATAATGTGGGTAGGACAGTTAGCAGTTCTGGATTGTATCGGATTCGAGGGGAATTTATGGCCGCT
TCAAGGTTGCTCAATTCTCGCTCATATCCTATCCCTTATGATTCATTATTTGAGGAGGCCCCAATTCCGCCTCGT
CGCCAGAAAAAAACGGATGAACAATCTAACAAAAAATTGTTGAATGAAACCGATGGTGACAATTCTGAGTGA
                                                                                                            GGTGACAATTCTGAGTGA
                                                                  TTTTTT.CCGCGG.GGTACC.TCACTCAGAATTGTCACC
                                                                                SacII KpnI
```

### 2.6. Cid I/BLUF Constructs

The second series of constructs is designed to insert the predicted active part of a BLUF signalling protein to the Cid I polymerase sequence. The locations for insertion were predicted to be functionally coupled to a Cid I polymerase activity regulating site.

Cid I(A) refers to amino acids 1-331 of SEQ ID NO. 9, and Cid I(B) refers to amino acids 332-405 of SEQ ID NO. 9.

```
Construct B - Cid I(A)-Linker-BLUF(cut)-Linker-Cid I(B)
Cid I(A)
SEQ ID NOs. 14 and 15
MNISSAQFIPGVHIVEEIEAEIHKNLHISKSCSYQKVPNSHKEFTKFCYEVYNEIKISDKEFKEKRAALDTLRLC
LKRISPDAELVAFGSLESGLALKNSDMDLCVLMDSRVQSDTIALQFYEELIAEGFEGKFLQRARIPIIKLTSDTK
NGFGASFQCDIGFNNRLAIHNTLLLSSYTKLDARLKPMVLLVKHWAKRKQINSPYFGTLSSYGYVLMVLYYLIHV
IKPPVFPNLLLSPLKQEKIVDGFDVGFDDKLEDIPPSQNYSSLGSLLHGFFRFYAYKFEPREKVVTFRRPDGYLT
KQEKGWTSATEHTGSADQIIKDRYILAIEDP
                            Apa I Hind III
AAAAAA.GCCCTT.GGGCCC.AAGCTT.
ATGAACATTTCTTCTGCA
ATGAACATTTCTTCTGCACAATTTATTCCTGGTGTTCACACAGTTGAAGAGATTGAGGCAGAAATTCACAAAAAT
TTACATATTTCAAAAAGTTGTAGCTACCAAAAGGTCCCTAATTCGCACAAGGAATTTACGAAGTTTTGCTATGAA
GTGTATAATGAGATTAAAATTAGTGACAAAGAGTTTAAAGAAAAGAGAGCGGCATTAGATACACTTCGGCTATGC
CTTAAACGAATATCCCCTGATGCTGAATTGGTAGCCTTTGGAAGTTTGGAATCTGGTTTAGCACTTAAAAATTCG
GATATGGATTTGTGCGTGCTTATGGATTCGCGCGTCCAAAGTGATACAATTGCGCTCCAATTCTATGAAGAGCTT
ATAGCTGAAGGATTTGAAGGAAAATTTTTACAAAGGGCAAGAATTCCCATTATCAAATTAACATCTGATACGAAA
AATGGATTTGGGGCTTCGTTTCAATGTGATATTGGATTTAACAATCGTCTAGCTATTCATAATACGCTTTTACTT
TCTTCATATACAAAATTAGATGCTCGCCTAAAACCCATGGTCCTTCTTGTTAAGCATTGGGCCAAACGGAAGCAA
ATCAACTCTCCTTACTTTGGAACTCTTTCCAGTTATGGTTACGTCCTAATGGTTCTTTACTATCTGATTCACGTT
ATCAAGCCTCCCGTCTTTCCTAATTTACTGTTGTCACCTTTGAAACAAGAAAAGATAGTTGATGGATTTGACGTT
GGTTTTGACGATAAACTGGAAGATATCCCTCCTTCCCAAAATTATAGCTCATTGGGAAGTTTACTTCATGGCTTT
TTTAGATTTTATGCTTATAAGTTCGAGCCACGGGAAAAGGTAGTAACTTTTCGTAGACCAGACGGTTACCTCACA
AAGCAAGAGAAAGGATGGACTTCAGCTACTGAACACACTGGATCGGCTGATCAAATTATAAAAGACAGGTATATT
CTTGCGATTGAAGATCCT
CTTGCGATTGAAGATCCTGGCGGCGGAGGG
CCCTCCGCCGCCAGGATCTTCAATCGCAAG
Linker
SEQ ID NOs. 18 and 19
GGGGS GGGGS GGGGS
ATTGAAGATCCT
GGCGGCGGAGGGAGTGGTGGCGGAGGGTCA
GGCGGCGGAGGGAGTGGTGGCGGAGGGTCAGGGGGCGGCGGCAGC
                              GGTGGCGGAGGGTCAGGGGGCGGCGGCAGCATGCTTACCACC
                              GGTGGTAAGCATGCTGCCGCCGCCCCCTGACCCTCCGCCACC
BLUF
SEQ ID NOs. 2 and 3
MLTTLIYRSHIRDDEPVKKIEEMVSIANRRNMQSDVTGILLFNGSHFFQLLEGPEEQVKMIYRAICQDPRHYNIV
ELLCDYAPA
GGCGGCGGCAGC
ATGCTTACCACCCTTATT
ATGCTTACCACCCTTATTTATCGTAGCCATATACGTGACGACGAACCTGTCAAAAAAATCGAAGAAATGGTTTCG
ATAGCAAATCGCAGGAACATGCAGTCTGACGTAACAGGGATCTTACTGTTTAATGGTTCTCATTTTTTCCAGCTT
CTGGAAGGTCCGGAAGAACAGGTTAAAATGATATATCGGGCTATATGCCAGGATCCACGGCACTATAATATTGTT
GAGCTGCTGTGCGATTACGCGCCTGCT
                  TGCGATTACGCGCCTGCTGGAGGAGGAGGA
                  TCCTCCTCCTCCAGCAGGCGCGTAATCGCA
Linker
SEQ ID NOs. 18 and 19
GGGGS GGGGS GGGGS
TACGCGCCTGCT
GGAGGAGGAGGATCCGGGGGAGGCGGTTCT
GGAGGAGGAGGATCCGGGGGAGGCGGTTCTGGCGGCGGGGGCAGC
                              GGGGGAGGCGGTTCTGGCGGCGGGGGCAGCTTCGAGATTTCA
                              TGAAATCTCGAAGCTGCCCCCGCCGCCAGAACCGCCTCCCCC
Cid I(B)
SEQ ID NOs. 16 and 17
FEISHNVGRTVSSSGLYRIRGEFMAASRLLNSRSYPIPYDSLFEEAPIPPRRQKKTDEQSNKKLLNETDGDNSE*
STOP
GGCGGGGGCAGC
TTCGAGATTTCACATAAT
TTCGAGATTTCACATAATGTGGGTAGGACAGTTAGCAGTTCTGGATTGTATCGGATTCGAGGGGAATTTATGGCC
GCTTCAAGGTTGCTCAATTCTCGCTCATATCCTATCCCTTATGATTCATTATTTGAGGAGGCCCCAATTCCGCCT
CGTCGCCAGAAAAAAACGGATGAACAATCTAACAAAAAATTGTTGAATGAAACCGATGGTGACAATTCTGAGTGA
                                                                                                                  GGTGACAATTCTGAGTGA
                                                                        TTTTTT.CCGCGG.GGTACC.TCACTCAGAATTGTCACC
                                                                                      SacII KpnI
```

2.7. Cid I/BLUF (Complete) Construct

The third construct is designed for verification. The domain assembly is reversed in comparison to the first two series: Cid I polymerase (amino acids 1-377 of SEQ ID NO. 9) is located at the N-terminal part, while BLUF makes the C-terminus of the fusion protein. Both domains feature unedited complete sequences.

Construct C—Cid I-Linker-BLUF

```
Cid I
SEQ ID NOs. 12 and 13
MNISSAQFIPGVHTVEEIEAEIHKNLHISKSCSYQKVPNSHKEFTKFCYEVYNEIKISDKEFKEKRAALDTLRLC
LKRISPDAELVAFGSLESGLALKNSDMDLCVLMDSRVQSDTIALQFYEELIAEGFEGKFLQRARIPIIKLTSDTK
NGFGASFQCDIGFNNRLAIHNTLLLSSYTKLDARLKPMVLLVKHWAKRKQINSPYFGTLSSYGYVLMVLYYLIHV
IKPPVFPNLLLSPLKQEKIVDGFDVGFDDKLEDIPPSQNYSSLGSLLHGFFRFYAYKFEPREKVVTFRRPDGYLT
KQEKGWTSATEHTGSADQIIKDRYILAIEDPFEISHNVGRTVSSSGLYRIRGEFMAASRLLNSRSYPIPYDSLFE
EA
              Apa I Hind III
AAAAAA.GGGCCC.AAGCTT.
ATGAACATTTCTTCTGCA
ATGAACATTTCTTCTGCACAATTTATTCCTGGTGTTCACACAGTTGAAGAGATTGAGGCAGAAATTCACAAAAAT
TTACATATTTCAAAAAGTTGTAGCTACCAAAAGGTCCCTAATTCGCACAAGGAATTTACGAAGTTTTGCTATGAA
GTGTATAATGAGATTAAAATTAGTGACAAAGAGTTTAAAGAAAAGAGAGCGGCATTAGATACACTTCGGCTATGC
CTTAAACGAATATCCCCTGATGCTGAATTGGTAGCCTTTGGAAGTTTGGAATCTGGTTTAGCACTTAAAAATTCG
GATATGGATTTGTGCGTGCTTATGGATTCGCGCGTCCAAAGTGATACAATTGCGCTCCAATTCTATGAAGAGCTT
ATAGCTGAAGGATTTGAAGGAAAATTTTTACAAAGGGCAAGAATTCCCATTATCAAATTAACATCTGATACGAAA
AATGGATTTGGGGCTTCGTTTCAATGTGATATTGGATTTAACAATCGTCTAGCTATTCATAATACGCTTTTACTT
TCTTCATATACAAAATTAGATGCTCGCCTAAAACCCATGGTCCTTCTTGTTAAGCATTGGGCCAAACGGAAGCAA
ATCAACTCTCCTTACTTTGGAACTCTTTCCAGTTATGGTTACGTCCTAATGGTTCTTTACTATCTGATTCACGTT
ATCAAGCCTCCCGTCTTTCCTAATTTACTGTTGTCACCTTTGAAACAAGAAAAGATAGTTGATGGATTTGACGTT
GGTTTTGACGATAAACTGGAAGATATCCCTCCTTCCCAAAATTATAGCTCATTGGGAAGTTTACTTCATGGCTTT
TTTAGATTTTATGCTTATAAGTTCGAGCCACGGGAAAAGGTAGTAACTTTTCGTAGACCAGACGGTTACCTCACA
AAGCAAGAGAAAGGATGGACTTCAGCTACTGAACACACTGGATCGGCTGATCAAATTATAAAAGACAGGTATATT
CTTGCGATTGAAGATCCTTTCGAGATTTCACATAATGTGGGTAGGACAGTTAGCAGTTCTGGATTGTATCGGATT
CGAGGGGAATTTATGGCCGCTTCAAGGTTGCTCAATTCTCGCTCATATCCTATCCCTTATGATTCATTATTTGAG
GAGGCC
TCATTATTTGAGGAGGCCGGAGGAGGAGGT
ACCTCCTCCTCCGGCCTCCTCAAATAATGA
Linker
SEQ ID NOs. 18 and 19
GGGGS GGGGS GGGGS
TTTGAGGAGGCC
GGAGGAGGAGGTAGCGGTGGCGGAGGGTCA
GGAGGAGGAGGTAGCGGTGGCGGAGGGTCAGGTGGCGGCGGGAGT
                              GGTGGCGGAGGGTCAGGTGGCGGCGGGAGTATGCTTACCACC
                              GGTGGTAAGCATACTCCCGCCGCCACCTGACCCTCCGCCACC
BLUF
SEQ ID NOs. 4 and 5
MLTTLIYRSHIRDDEPVKKIEEMVSIANRRNMQSDVTGILLFNGSHFFQLLEGPEEQVKMIYRAICQDPRHYNIV
ELLCDYAPARRFGKAGMELFDLRLHERDDVLQAVFDKGTSKFQLTYDDRALQFFRTFVLATEQSTYFEI*STOP
GGCGGCGGGAGT
ATGCTTACCACCCTTATT
ATGCTTACCACCCTTATTTATCGTAGCCATATACGTGACGACGAACCTGTCAAAAAAATCGAAGAAATGGTTTCG
ATAGCAAATCGCAGGAACATGCAGTCTGACGTAACAGGGATCTTACTGTTTAATGGTTCTCATTTTTTCCAGCTT
CTGGAAGGTCCGGAAGAACAGGTTAAAATGATATATCGGGCTATATGCCAGGATCCACGGCACTATAATATTGTT
GAGCTGCTGTGCGATTACGCGCCTGCTCGCCGTTTTGGCAAAGCGGGAATGGAATTATTTGATTTGCGCCTGCAC
GAGCGAGATGACGTTTTACAGGCCGTATTCGACAAAGGCACATCAAAATTTCAGCTAACTTATGATGACAGAGCG
CTACAATTTTTTCGTACTTTTGTCCTTGCAACCGAACAATCAACCTATTTCGAGATCTAA
                                                                                    ACCTATTTCGAGATCTAA
                                  TTTTTT.TCT.CCGCGG.GGTACC.TTAGATCTCGAAATAGGT
                                                        SacII KpnI
```

2.8. BLUF/Cid I/GFP (Preparation) Construct

The fourth series of constructs is designed to insert the predicted active part of a BLUF signalling protein to the Cid I polymerase sequence. To add an additional internal control mechanism a second BLUF domain together with a linker structure is attached to GFP. The second BLUF domain is located at the C-terminus of the resulting fusion protein and prepares expression in a GFP-containing expression vector system. The GFP domain sequence is already integrated into the chosen expression vector system.

Construct D—BLUF(Cut)-Linker 1-Cid I-Linker 2-BLUF(Cut Long)-GFP(Prepare)

```
BLUF
SEQ ID NOs. 2 and 3
MLTTLIYRSHIRDDEPVKKIEEMVSIANRRNMQSDVTGILLFNGSHFFQLLEGPEEQVKMIYRAICQDPRHYNIV
ELLCDYAPA
                                  Pst I Bgl II
AAAAAA.CGCGCGCGC.CTGCAG.AGATCT.
ATGCTTACCACCCTTATT
ATGCTTACCACCCTTATTTATCGTAGCCATATACGTGACGACGAACCTGTCAAAAAAATCGAAGAAATGGTTTCG
ATAGCAAATCGCAGGAACATGCAGTCTGACGTAACAGGGATCTTACTGTTTAATGGTTCTCATTTTTTCCAGCTT
CTGGAAGGTCCGGAAGAACAGGTTAAAATGATATATCGGGCTATATGCCAGGATCCACGGCACTATAATATTGTT
GAGCTGCTGTGCGATTACGCGCCTGCT
                  TGCGATTACGCGCCTGCTGGTGGTGGTGGT
                  ACCACCACCACCAGCAGGCGCGTAATCGCA
Linker
SEQ ID NOs. 18 and 19
GGGGS GGGGS GGGGS
TACGCGCCTGCT
GGTGGTGGTGGTTCTGGTGGTGGTGGTAGT
GGTGGTGGTGGTTCTGGTGGTGGTGGTAGTGGCGGAGGAGGGAGC
                              GGTGGTGGTGGTAGTGGCGGAGGAGGGAGCAGCTACCAAAAG
                              CTTTTGGTAGCTGCTCCCTCCTCCGCCACTACCACCACCACC
Cid I
SEQ ID NOs. 10 and 11
SYQKVPNSHKEFTKFCYEVYNEIKISDKEFKEKRAALDTLRLCLKRISPDAELVAFGSLESGLALKNSDMDLCVL
MDSRVQSDTIALQFYEELIAEGFEGKFLQRARIPIIKLTSDTKNGFGASFQCDIGFNNRLAIHNTLLLSSYTKLD
ARLKPMVLLVKHWAKRKQINSPYFGTLSSYGYVLMVLYYLIHVIKPPVFPNLLLSPLKQEKIVDGFDVGFDDKLE
DIPPSQNYSSLGSLLHGFFRFYAYKFEPREKVVTFRRPDGYLTKQEKGWTSATEHTGSADQIIKDRYILAIEDPF
EISHNVGRTVSSSGLYRIRGEFMAASRLLNSRSYPIPYDSLFEEAPIPPRRQKKTDEQSNKKLLNETDGDNSE*S
TOP
GGAGGAGGGAGC
AGCTACCAAAAGGTCCCT
AGCTACCAAAAGGTCCCTAATTCGCACAAGGAATTTACGAAGTTTTGCTATGAAGTGTATAATGAGATTAAAATT
AGTGACAAAGAGTTTAAAGAAAAGAGAGCGGCATTAGATACACTTCGGCTATGCCTTAAACGAATATCCCCTGAT
GCTGAATTGGTAGCCTTTGGAAGTTTGGAATCTGGTTTAGCACTTAAAAATTCGGATATGGATTTGTGCGTGCTT
ATGGATTCGCGCGTCCAAAGTGATACAATTGCGCTCCAATTCTATGAAGAGCTTATAGCTGAAGGATTTGAAGGA
AAATTTTTACAAAGGGCAAGAATTCCCATTATCAAATTAACATCTGATACGAAAAATGGATTTGGGGCTTCGTTT
CAATGTGATATTGGATTTAACAATCGTCTAGCTATTCATAATACGCTTTTACTTTCTTCATATACAAAATTAGAT
GCTCGCCTAAAACCCATGGTCCTTCTTGTTAAGCATTGGGCCAAACGGAAGCAAATCAACTCTCCTTACTTTGGA
ACTCTTTCCAGTTATGGTTACGTCCTAATGGTTCTTTACTATCTGATTCACGTTATCAAGCCTCCCGTCTTTCCT
AATTTACTGTTGTCACCTTTGAAACAAGAAAAGATAGTTGATGGATTTGACGTTGGTTTTGACGATAAACTGGAA
GATATCCCTCCTTCCCAAAATTATAGCTCATTGGGAAGTTTACTTCATGGCTTTTTTAGATTTTATGCTTATAAG
TTCGAGCCACGGGAAAAGGTAGTAACTTTTCGTAGACCAGACGGTTACCTCACAAAGCAAGAGAAAGGATGGACT
TCAGCTACTGAACACACTGGATCGGCTGATCAAATTATAAAAGACAGGTATATTCTTGCGATTGAAGATCCTTTC
GAGATTTCACATAATGTGGGTAGGACAGTTAGCAGTTCTGGATTGTATCGGATTCGAGGGGAATTTATGGCCGCT
TCAAGGTTGCTCAATTCTCGCTCATATCCTATCCCTTATGATTCATTATTTGAGGAGGCCCCAATTCCGCCTCGT
CGCCAGAAAAAAACGGATGAACAATCTAACAAAAAATTGTTGAATGAAACCGATGGTGACAATTCTGAGTGA
GGTGACAATTCTGAGTGAGGCGGAGGAGGT
ACCTCCTCCGCCTCACTCAGAATTGTCACC
Linker
SEQ ID NOs. 18 and 19
GGGGS GGGGS GGGGS
AATTCTGAGTGA
GGCGGAGGAGGTAGCGGTGGCGGAGGGTCA
GGCGGAGGAGGTAGCGGTGGCGGAGGGTCAGGTGGTGGGGGAAGT
                              GGTGGCGGAGGGTCAGGTGGTGGGGGAAGTATGCTTACCACC
                              GGTGGTAAGCATACTTCCCCCACCACCTGACCCTCCGCCACC
BLUF
SEQ ID NOs. 6 and 7
MLTTLIYRSHIRDDEPVKKIEEMVSIANRRNMQSDVTGILLENGSHFFQLLEGPEEQVKMIYRAICQDPRHYNIV
ELLCDYAPARRFGKAGMELFDLRLHERDDVLQAVFDKGTSKFQLTYDDRA
GGTGGGGGAAGT
ATGCTTACCACCCTTATT
ATGCTTACCACCCTTATTTATCGTAGCCATATACGTGACGACGAACCTGTCAAAAAAATCGAAGAAATGGTTTCG
ATAGCAAATCGCAGGAACATGCAGTCTGACGTAACAGGGATCTTACTGTTTAATGGTTCTCATTTTTTCCAGCTT
CTGGAAGGTCCGGAAGAACAGGTTAAAATGATATATCGGGCTATATGCCAGGATCCACGGCACTATAATATTGTT
GAGCTGCTGTGCGATTACGCGCCTGCTCGCCGTTTTGGCAAAGCGGGAATGGAATTATTTGATTTGCGCCTGCAC
GAGCGAGATGACGTTTTACAGGCCGTATTCGACAAAGGCACATCAAAATTTCAGCTAACTTATGATGACAGAGCG
                                                                                                                  ACTTATGATGACAGAGCG
                                                                        AAAAAA.CTCGAG.AAGCTT.CGCTCTGTCATCATAAGT
                                                                                      Xho I Hind III
```

2.9. BLUF/GFP (Preparation) Construct

The fifth series of constructs is designed to insert the predicted active part of a BLUF signalling protein to the GFP reporter domain sequence. While BLUF makes the N-terminus of the fusion protein, the GFP domain sequence is already integrated into the chosen expression vector system.

The predicted change in GFP activity level is shown in FIG. 9.

Construct E—BLUE (Cut)-GFP (Prepare)

```
BLUF
SEQ ID NOs. 2 and 3
MLTTLIYRSHIRDDEPVKKIEEMVSIANRRNMQSDVTGILLFNGSHFFQLLEGPEEQVKMIYRAICQDPRHYNIV
ELLCDYAPA
              Pst I Bgl II
AAAAAA.CTGCAG.AGATCT.
ATGCTTACCACCCTTATTTATCGTAGC
ATGCTTACCACCCTTATTTATCGTAGCCATATACGTGACGACGAACCTGTCAAAAAAATCGAAGAAATGGTTTCG
ATAGCAAATCGCAGGAACATGCAGTCTGACGTAACAGGGATCTTACTGTTTAATGGTTCTCATTTTTTCCAGCTT
CTGGAAGGTCCGGAAGAACAGGTTAAAATGATATATCGGGCTATATGCCAGGATCCACGGCACTATAATATTGTT
GAGCTGCTGTGCGATTACGCGCCTGCTCGCCGTTTTGGCAAAGCGGGAATGGAATTATTTGATTTGCGCCTGCAC
GAGCGAGATGACGTTTTACAGGCCGTATTCGACAAAGGCACATCAAAATTTCAGCTAACTTATGATGACAGAGCG
                                                                                                TTTCAGCTAACTTATGATGACAGAGCG
                                                      AAAAAA.CTCGAG.AAGCTT.CGCTCTGTCATCATAAGTTAGCTGAAA
                                                                    Xho I Hind III
```

### Example 2 Bacterial Expression of BLUF-GFP and BLUF-Cid Constructs

BLUF-domain (the sensor for Blue Light Using FAD) is a novel blue light photoreceptor, identified in 2002 and it is found in more than 50 different proteins. These proteins are involved in various functions, such as photophobic responses (e.g. PAC protein—Euglena gracilis, Slr1694—Synechocystis sp.) and regulation of transcription (e.g. AppA protein ˜Rhodobacter sphaeroides, Blrp—E. coli). The proteins containing BLUF or similar domain are also found in Klebsiella pneumoniae, Naegleria gruberi, Acinetobacter baylyi and many other organisms. The molecular mechanism of BLUF-domain is very sophisticated. It converts the light signal to the biological information, following the conformational changes of the photoreceptor. Those changes are then recognized by other protein modules that traverse the signal to the downstream machineries. This type of light signal transduction mechanism was specifically modified in each organism during the evolution, to allow the adaptation for the different environmental conditions.

Main Aim:

To produce BLUF and BLUF-Cid1 in E. coli expression system. See FIG. 10.

Tools:

A circular DNA plasmid pPK-CMV-F1 vector with inserted BLUF domain with GFP on C-terminus (BLUF-GFP construct, see FIG. 11)/A circular DNA plasmid pUC57 with inserted BLUF domain with Cid1 polymerase in short or long version [BLUF-Cid1(cut) construct, BLUF-Cid1(long) construct, see FIG. 13]. Between the sequence of BLUF and Cid1 in both constructs was used linker GGGGS GGGGS GGGGS, which does not affect the folding of the fusion protein partners.

Used DNA for the BLUF: gene ycgF (accession number AAC74247.3, see SEQ ID NO. 1.) from the E. coli strain DH5-α,

amplicon from 1-375 nt (125 AA), see SEQ ID NOs. 6 and 7.

Used DNA for the Cid1: poly(A) polymerase Cid1 (accession number NP_594901) from the Schizosaccharomyces pombe [972h-]

SEQ ID NO. 9.

Used Primers:

Constructs BLUF-GFP:

BLUF-GFP FW:

SEQ ID NO. 20

AAAAAACTGCAGAGATCTATGCTTACCACCCTTATTTATCGTAGC

(including restriction sites for PstI and BglII)

BLUF-GFP RV:

SEQ ID NO. 21

TTTTTTGAGCTCTTCGAAGCGAGACAGTAGTATTCAATCGACTTT

(including restriction sites for XhoI and HindIII)

After amplification of BLUF domain, PCR product was digested and ligated into the pPK-CMV-F1 vector and ligation mix was used for the transfection of bacteria. As a host strain E. coli strain DH5-α and E. coli strain Rosetta (chemical transformation) were used.

For the preparation of the BLUF-Cid1 constructs, the commercial service (GenScript) was used to prepare the vectors with inserted sequences. The plasmids were used for chemical transformation of E. coli strain M15.

After transformation, bacteria with BLUF-GFP construct were cultured in Lysogeny broth (LB) and the protein was expressed using 1 mM Isopropyl β-D-1-thiogalactopyranoside (IPTG) (FIGS. 12 C and E). As a positive control, bacteria transformed with GFP only were used (FIG. 12 B). As a negative control, non-induced bacteria were used (FIGS. 12 C and D).

To assess whether the GFP is expressed under the control of BLUF domain, bacteria were cultivated under two different conditions (in dark or in light) for 16 and 24 hours with 1 mM IPTG on LB agar with selective antibiotics. The fluorescence of live bacteria was visualized with a fluorescent microscope. The results suggest that after 16 hours of incubation, the bacteria were fluorescent under the light conditions, but not the dark conditions (FIGS. 12 B and D). After 24 hours, the difference between the intensity of fluorescence under light or dark condition was not significant (FIG. 4, panel F and H).

Subsequently, the bacteria were harvested and lysed under the native conditions with native lysis buffer with 1 mg/ml of lysozyme and protease inhibitor cocktail, with short sonification (3×10 sec cycles). The cell debris was removed by centrifugation and the supernatant contains proteins were separated by PAGE under reducing conditions. As seen in FIG. 13 A, all the recombinant proteins were overexpressed. The BLUF domain itself was observed as a low-molecular weight protein (FIG. 13 A, lanes 2 and 6), while BLUF-Cid1 constructs were observed as a low-molecular weight component with MW approximately 13 kDa and two high-MW components, approximately 45 kDa and 58 kDa (FIG. 13 A, lanes 3 and 4). Predicted molecular weight for recombinant BLUF domain fragment is 10.2 kDa, for the Cid1 fragment 42.7 kDa and for the BLUF-Cid1 construct 54.2 kDa (predicted by Geneious software).

The BLUF-Cid1 construct contains also the HIS-tag for easier purification, whereas the vector containing BLUF-GFP insert did not contain any tag. Accordingly, the presence of the BLUF-Cid1 construct in the lysate was also detected by western blot. In short, the lysate of BLUF-GFP (as a negative control), BLUF-Cid1 (cut) and BLUF-Cid1(long) was trickled onto the nitrocellulose membrane, and after drying, the membrane was blocked with 2% bovine albumin to remove the non-specific interactions. Subsequently, membrane was hybridized with Ni-HRP conjugate and the presence of His-tagged proteins were visualized. In the case of BLUF-GFP, any protein was detected (FIG. 13 B, spot 1), while both constructs BLUF-Cid1 was detected (FIG. 13 B, spots 2 and 3).

### Example 3 Detection of Polymerase Activity of BLUF-CidI

The GFP control construct series allows monitoring differences in activity for expressed fusion proteins. While the GFP control vector shows fluorescence activity at the expected standard level, the BLUF-GFP fusion constructs feature elevated activity levels both at UV lighting (FIG. 9A) and a combination of UV and daylight (FIG. 9B).

Detailed functional proof of the observed correct fluorescence activity of the constructs requires polymerase or kinase activity monitoring using a fluorescent oligonucleotide (FIG. 5). This was achieved for T4 polynucleotide kinase.

In addition, this was achieved with different Cid I polymerase constructs,
i.e. synthesis of different nucleotide sequences and control by BLUF domains; confirmation for correctly synthesized sequences after switching the construct to on —using molecular beacons.

as well as direct monitoring of fluorescence in read-out BLUF-GFP constructs
i.e. switching off the BLUF domain by blue light stops then fluorescence; documented by light microscopy.

1. Molecular Beacon Assay

The molecular beacon uses for CidI Polymerase activity monitoring an RNA beacon as template, the synthesized polyU from the light-gated activated (by blue light) Cid I polymerase opens up the beacon structure and fluorescence changes. Molecular beacons are advantageous in many applications to detect nucleic acid synthesis and quantify it. The stem-loop structure of a molecular beacon may open up or change and provides a competing reaction for probe-target hybridization. FIGS. 20 A and B are drawn and given according to Tsourkas et al., 2003 and illustrate the general technique: FIG. 20 A shows design and structural parameters of molecular beacons. FIG. 20 B shows that molecular beacons in solution can have three phases: bound to target, closed and random coil.

We then generated the following beacons and oligonucleotides/primers with fluorophor TAMRA and the quencher BHQ2 for our experiments:

3.1 Control Experiments and Positive Controls Using DNA as Well as Klenow Fragment:

Beacon MB_1 (DNA):

SEQ ID NO. 23

5′-TAMRA-CCTCGTGTCTTGTACTTCCCGTCCGAGG-BHQ2-3′

This required the corresponding “Oligo_A” (DNA):

SEQ ID NO. 24

5′-GACGGGAAGTACAAGACAC-3′

For the experiments with the Klenow-fragment, the primer “Oligo_B” (DNA) was used:

5′-GACGGGAAG-3′

3.2 for the Experiments with the Cid1-Poly-U-Polymerase we Used the Following Oligonucleotides:

Beacon MB_2_Poly-U (DNA):

SEQ ID NO. 25
5′-TAMRA-CCTCAAAAAAAAAAAAAAACGCGGCTGAGG-BHQ2-3′

With the corresponding oligonucleotide to open the beacon (positive control) “Oligo_PUr” (RNA):

SEQ ID NO. 26

5′-GCCGCGUUUUUUUUUUUUUUU-3′

For the activity monitoring of the Cid1-Polymerase, the primer “Oligo_PriUr” (RNA) was used:

SEQ ID NO. 27

5′-GCCGCGUUUU-3′

FIGS. 20 C and D show the structure of the beacon MB1 alone (C) and with the oligonucleotide (D), so the beacon in the target bound state; also measured when the Klenow polymerase is active).

FIGS. 20 E and F show the structure of the beacon MB2 alone and with the oligonucleotide (so beacon in the target bound state; also measured when the CidI polymerase is active).

We hence generated a molecular beacon for CidI polymerase activity monitoring so that it works and opens up to bind to the target as soon as there is polyU synthesized by the CidI polymerase, and then quencher and fluorophore are separated. In several independent experiments efficient polyU synthesis was observed only if the CidI polymerase construct was switched on and active. Moreover, this could only be observed for the blue-light gated form of the CidI polymerase construct when blue light was there and stopped, when the blue-light was switched off

2. Light Microscopy Test

Another example is direct monitoring of fluorescence in read-out BLUF-GFP constructs i.e. switching off the BLUF domain by blue light stops then fluorescence; documented by light microscopy (FIG. 12).

Here one of the imbedded molecular components was tested, using a light-gated GFP monitoring construct. There is light-gated (blue light mediated) control of GFP fluorescence. The different panels show in detail how only blue light/daylight allows full GFP fluorescence to develop whereas no switching on of the blue light mediating BLUF domain strongly reduces obtained GFP fluorescence.

### Example 4 Bacterial Expression of Nanocellulose

Nanocellulose is an emerging multipurpose biomaterial, which can be obtained from the two natural sources: from wood or microorganisms. The wooden nanocellulose is made from wood pulp, from which the non-cellulose components are removed. The purified pulp is then homogenized and the mixture is separated to cellulose fibers, which are then formed to paste, crystals or spaghetti-like fibers. Bacterial nanocellulose for the industrial and medical usage is prepared mostly by fermentation of Gluconacetobacter xylinus, but there are more species able to produce the cellulose, such as Achromobacter, Sarcina, Pseudomonas and Dickeya. Bacterial nanocellulose has several interesting features, such as unique nanostructure, high capacity to absorb water, high level of polymerization, followed by high mechanical strength and crystallinity, which categorize the nanocellulose to the group of potential ecological material for the 21th century.

Nanocellulose can be used in various fields of industry; pharmaceutical, food production, textile, electronic, cosmetic and many more areas.

The recombinant DNA technology is routinely used in agriculture, food industry and medicine, but currently there is a new challenge—to produce the new biomaterials with desired properties. The materials, which have their origin in nature but are used in bioengineering are called ‘recombinamers’ and we believe, that bacterial nanocellulose can be produced also in this manner.

Main Aim:

To produce nanocellulose in E. coli expression system.

Tools:

A circular DNA plasmid pQE-30-mCHERRY-GFP vector with inserted BcsA/BcsB unit, see FIGS. 21 A and B.

Used DNA for BcsA: gene bcsA—Cellulose synthase catalytic subunit [UDP-forming] (accession number AAB18510.1.) from the E. coli strain DH5-11, amplicon from 34-2610 nt (858 AA).

See SEQ ID NO. 32 for the full length amino acid sequence (872 aa), as shown in Database: UniProt/SWISS-PROT, Entry: BCSA_ECOLI.

Used DNA for the BcsB: gene bcsB—Cyclic di-GMP-binding protein (accession number AAB18509.1.) from the E. coli strain DH5-α, amplicon from 82-2331 nt (750 AA). See SEQ ID NO. 33 for the full length amino acid sequence (779 aa), as shown in Database: UniProt/SWISS-PROT, Entry: BCSB_ECOLI.

Used Primers:

BcsA-GFP Construct:

BcsA F:

SEQ ID NO. 28

TATGGATCCCCGGTCAACGCGCGGCTTATC

(including restriction site for BamHI)

BcsA R:

SEQ ID NO. 29

TCTGTCGACAGCCAAAGCCTGATCCGATGG

(including restriction site for SalI)

BcsB-mCHERRY Construct

BcsB F:

SEQ ID NO. 30

CCTGGTACCGCAACGCAACCACTGATCAAT

(including restriction site for KpnI)

BcsB R:

SEQ ID NO. 31

ATGCTCAGCATCCGGGTTAAGACGACGACG

(including restriction site for BlpI)

After amplification of BcsA and BcsB coding sequences (FIG. 14), PCR products was digested and ligated into the pQE-30 GFP or pQE-30 mCHERRY plasmid and ligation mixes were used for the transfection of bacteria. As a host strain was used E. coli strain M15 (chemical transformation).

After transformation, bacteria with constructs were cultured LB media and the proteins were expressed using 1 mM IPTG. The expressed proteins were visualized by fluorescent microscope. The bacteria with the construct BcsA emitted green fluorescence (FIG. 15 A), while bacteria transfected by BcsB red fluorescence (FIG. 15 B).

Transfected bacteria were after time-dependent induction (6, 21, 30 and 45 hrs) harvested and lysed under the denaturating conditions using 8M urea and purified by Ni-NTA resin. The BcsA construct was probably cleaved during the lysis so we didn't get any results on PAGE (predicted MW for the BcsA is 99.7 kDa, BcsA-GFP—130 kDa, GFP—30 kDa), but the BcsB was significantly overexpressed (predicted MW for the BcsB is 86 kDa, BcsB-mCHERRY—112 kDa, mCHERRY 26 kDa) (FIG. 16). As the further experiment, BcsA and BcsB will be fused with overlapping primer to obtain one molecule with His-tag, without the fluorescent reporter.

As an initial experiment to test the properties of bacterial nanocellulose, we tried to prepare the fluorescent nanocellulose. Bacterial nanocellulose was kindly provided by Dr. Kralish (JeNaCell, Germany). The recombinant protein mCHERRY-GFP was prepared from the in-house modified plasmid pQE-30 GFP-mCHERRY and after purification was hybridized with nanocellulose for 24 hrs in 4° C. Fluorescence was asses by fluorescence microscope (100×, FIG. 17, FIG. 18).

### Example 5 3D Printing

We furthermore can show that our nanocellulose composite with all its components is a suitable object to be produced by 3D printing technology.

A standard printer can be used for this, however, as is known for 3D printing of biological objects such as tissues or cells, the temperature and medium has to be suitably chosen (citation) (FIG. 19A).

We furthermore can show that our nanocellulose composite with all its components is a suitable object to be produced by 3D printing technology (scheme: FIG. 19A). A standard 3D printer can be used for this (Makerbot: Replicator 2×; FIG. 19B; Pettis et al., 2012), however, as is known for 3D printing of biological objects such as tissues or cells, the temperature and medium has to be suitable chosen.

Objective or Main Aim:

The nanocellulose chip is demanding to produce using only molecular biology techniques, it is not easy to modify and the numbers produced are low. Furthermore, the biotechnological synthesis process differs clearly from typical production methods in computer industry (silicon-wafers) which are more convenient to handle, faster and easy to modify.

Solution:

We present here a 3D printer variant for the production of nanocellulose chips, which allows their efficient production in high numbers. This enhances the quality of the nanocellulose chips obtained, additives which serve to conserve and protect the smart card and the integrated DNA can be easily added. Moreover, specific smart molecules are particularly well suitable to serve as micro printers (smart actuators) and are easily integrated into the chip card by this approach.

### Embodiment Example

Accordingly, this invention explains how these valuable nanocellulose chips are produced with the help of a 3D printer fast, convenient, flexible and at low cost (for a review of 3D printers see Scheufens M, 2014). For this a specific form of a 3D printer is used: a specific modification of the nanocellulose to become printable (printer matrix) and a specific type of additives in the printing matrix (proteins, DNA, fluorophores, nucleotides and chemicals; specific protein engineering constructs, as described herein). Together this achieves the final product of the improved nanocellulose chip in high quality and high numbers.

Specific Properties:

3D printer (basic scheme in FIG. 19A): A basic version uses for printing the PolyJet printer (ink jet principle). It was invented by the Israel-based enterprise PolyJet that fused 2012 with Stratasys Ltd. This printer can use at the same time several “inks”, for instance plastics. It is particularly suited for our application, printing of biomolecules and nanocellulose.

There are already large-sized printers, for instance the Voxeljet till 4×2×1 meter size (voxeljet AG, Friedberg, Germany). We recommend for the invention in order to achieve smart chips from nanocellulose by 3D printing the following three printer types (3D):
I. melting printing;
II. photo polymerization: UV light polymerizes liquid, light-fragile substance. Patented by Chuck Hulls (1984), 3D systems which is world-wide market leader: EnvisionTEC. This allows high accuracy printing with layers of 15 micro meters),
III. A 3rd approach is Laser sintering (this is expensive, but very good to use for fast and simple conservation of information stored in our smart cards made of nanocellulose).

Basic Matrix:

In particular suitable is pure nanocellulose as wells bacterial cellulose (BC)/polycaprolactone (PCL) nanocomposite films. For these the production with hot compression is known (Figueiredo et al., 2015) as well as composite films from poly(vinyl ethanol) and bifunctional coupled cellulose nano crystals (Sirvio et al., 2015) as well as polylactid latex/nanofibrillated cellulose bio-nanocomposites (Larsson et al., 2012).

Further additives contain pure DNA for information storage or as substrate. It can furthermore be used as adaptor DNA or oligo-macrame (Lv et al., 2015) or as pore-membrane designer (Langecker et al., 2012).

Specific constructs, suitable for our invention: PolyU CidI Polymerase (with BLUF-domain or light-gated control), PolyA CidI Polymerase (or similarly controlled), or specifically modified, as well as further (modified) polymerases, light-gated controlled for preference, as well as (similarly modified) exonucleases, furthermore (light-gated), GFP-constructs and other fluorescent proteins, as well as different DNA molecules (with modifications).

Optimize Printing:

The optimal application and concentration of the mixture and the optimal temperature are important.

### Further Embodiments

Printing: An alternative to ink jet printing is the makerbot replicator printer (see FIG. 19 B; Pettis et al., 2012). Other 3D printer types may be adapted, such as the sinter printer (here additives have to be made heat stable) and a printer with melting and printed layers (Adrian Bowyer, University of Bath 2005). These can partly print themselves as they contain in their construction a large fraction of printed parts.
The printed chips are an important interface to other computer chips (produced from semiconductor industry) and this as printed circuits; for interfacing to these chips our nanocellulose composite chips use light or electronic properties.
The added biomolecules, in particular the actuators (preferentially light-gated), support printing as micro-printers and for micro patterning and for the molecular translation (DNA, RNA) of genes (or parts thereof) and further enhance the functionality of the printed nanocellulose composite towards an “universal constructor”, i.e. a high flexible nanomachine for the production and the printing of information processing circuits.
Long term storage of DNA by incorporation of silica glass particles can be easily achieved by our 3D printing approach and the above listed 3D printers, an important difference to demanding chemistry proposed earlier (Glass et al., 2015).

The features disclosed in the foregoing description, in the claims and/or in the accompanying drawings may, both separately and in any combination thereof, be material for realizing the invention in diverse forms thereof.

### REFERENCES

Allen M J, Schoonmaker J E, Bauer T W, Williams P F, Higham P A, Yuan H A: Preclinical evaluation of a poly(vinyl alcohol)hydrogel implant as a replacement for the nucleus pulposus. Spine 29: 515-523 (2004).
Barnhardt D, Koek W, Juchem T, Hampp N, Coupland J, et al. Bacteriorhodopsin as a high-resolution, high-capacity buffer for digital holographic measurements. Meas Sci Technol. 2004; 15:639-646.
Bitrian, M., González, R. H., Paris, G., Hellingwerf, K. J., & Nudel, C. B. Blue-light-dependent inhibition of twitching motility in Acinetobacter baylyi ADP1: additive involvement of three BLUF-domain-containing proteins. Microbiology, (2013). 159(9), 1828-1841.
Buga A M, Scholz C J, Kumar S, Herndon J G, Alexandra D, Cojocaru G R, Dandekar T, Popa-Wagner A. Identification of new therapeutic targets by genome-wide analysis of gene expression in the ipsilateral cortex of aged rats after stroke. PLoS One. 2012; 7(12):e50985. doi: 10.1371/journal.pone.0050985.
Cheng Y H, Yang S H, Su W Y, Chen Y C, Yang K C, Cheng W T, Wu S C, Lin F H. Thermosensitive chitosan-gelatin-glycerol phosphate hydrogels as a cell carrier for nucleus pulposus regeneration: an in vitro study. Tissue Eng Part A. 2010 February; 16(2):695-703. doi: 10.1089/ten. TEA.2009.0229.
Chou A I, Akintoye S O, Nicoll S B. Osteoarthritis Cartilage. Photo-crosslinked alginate hydrogels support enhanced matrix accumulation by nucleus pulposus cells in vivo. 2009 October; 17(10):1377-84. doi: 10.1016/j.joca.2009.04.012. Epub 2009 May 4.
Church, G. M., Gao, Y., & Kosuri, S. (2012). Next-generation digital information storage in DNA. Science, 337(6102), 1628-1628.
Conrad K S, Manahan C C, Crane B R. Photochemistry of flavoprotein light sensors. Nat Chem Biol. 2014 October; 10(10):801-9.
Esa F., Tasirin S. M., Rahman N. A. (2014) Overview of Bacterial Cellulose Production and Application. Agriculture and Agricultural Science Procedia Volume 2, Pages 113-119.
Ferguson, W (2012) Why wood pulp is world's new wonder material. New Scientist, Issue 2878, posting http://www.newscientist.com/article/mg21528786.100-why-wood-pulp-is-worlds-new-wonder-material.html#.VMTREi5DAbI.
Figueiredo A R et al. In situ synthesis of bacterial cellulose/polycaprolactone blends for hot pressing nanocomposite films production. Carbohydr Polym. 2015 Nov. 5; 132:400-8. doi: 10.1016/j.carbpol.2015.06.001. Epub 2015 Jun. 8.
Fricke F, Malkusch S, Wangorsch G, Greiner J F, Kaltschmidt B, Kaltschmidt C, Widera D,
Dandekar T, Heilemann M. Quantitative single-molecule localization microscopy combined with rule-based modeling reveals ligand-induced TNF-R1 reorganization toward higher-order oligomers. Histochem Cell Biol. 2014 July; 142(1):91-101. doi: 10.1007/s00418-014-1195-0.
Goldman, N., Berton, P., Chen, S., Dessimoz, C., LeProust, E. M., Sipos, B. & Birney, E. Towards practical, high-capacity, low-maintenance information storage in synthesized DNA. Nature 494, 77-80 (2013).
Gomelsky, M. and Klug, G., BLUF: a novel FAD-binding domain involved in sensory transduction in microorganisms Trends Biochem. Sci. 27, (2002), 497-500.
Grass, R. N., Heckel, R., Puddu, M., Paunescu, D., & Stark, W. J. (2015). Robust Chemical Preservation of Digital Information on DNA in Silica with Error-Correcting Codes. AngewandteChemie International Edition, 54(8), 2552-2555.
Gruber Helen E., Gretchen L. Hoelscher, Kelly Leslie, Jane A. Ingram, Edward N. Hanley Jr. Three-dimensional culture of human disc cells within agarose or a collagen sponge: assessment of proteoglycan production. Biomaterials. 2006 January; 27(3): 371-6.
Halabi, N., Rivoire, O., Leibler, S. & Ranganathan, R. Protein sectors: evolutionary units of three-dimensional structure. Cell 138, 774-786, doi:10.1016/j.ce11.2009.07.038 (2009).
Imhof, M., Rhinow, D., Hampp, N. Two-photon polarization data storage in bacteriorhodopsin films and its potential use in security applications. Appl. Phys. Lett., 104, 081921 (2014).
Iseki, M., Matsunaga, S., Murakami, A., Ohno, K., Shiga, K., Yoshida, K., Sugai, M., Takahashi, T., Hori, T. and Watanabe, M., A blue-light-activated adenylyl cyclase mediates photoavoidance in Euglena gracilis. Nature 415, (2002), 1047-1051.
Klemm D et al. Nanocelluloses: A new family of nature-based materials. Angew. Chemie Int. Edition (2011) 50(24), 5438-5466.
Kralisch, D. (2015) Nanozellulose -ein bakteriell erzeugtes Hochleistungspolymer. Spektrum der Wissenschaften January 2015, p. 78-84.
Krüger, A. (2014) Die Entwicklung regenerativer Implantatmatrices auf der Basis von Kollagen Typ I zur Anwendung bei degenerativen Bandscheibenerkrankungen. PhD thesis.
Krueger B, Friedrich T, Förster F, Bernhardt J, Gross R, Dandekar T. Different evolutionary modifications as a guide to rewire two-component systems. Bioinform Biol Insights. 2012; 6:97-128. doi: 10.4137/BBI.S9356.
Langecker M et al. Synthetic lipid membrane channels fixated by designed DNA nanostructures. Science 2012, vol. 338, issue 6109, pp. 932-936.
Larsson K et al. Polylactide latex/nanofibrillated cellulose bionanocomposites of high nanofibrillated cellulose content and nanopaper network structure prepared by papermaking route. Journal of Applied Polymer Science (2012). Available online, DOI: 10.1002/app.36413.
Lee, J., Natarajan, M., Nashine, V. C., Socolich, M., Vo, T., Russ, W. P., et al. Surface sites for engineering allosteric control in proteins. Science 322, 438-442, doi:10.1126/science.1159052 (2008).
Lee K I, Moon S H, Kim H, Kwon U H, Kim H J, Park S N, Suh H, Lee H M, Kim H S, Chun H J, Kwon I K, Jang J W. Tissue engineering of the intervertebral disc with cultured nucleus pulposus cells using atelocollagen scaffold and growth factors. Spine (Phila Pa. 1976). 2012 Mar. 15; 37(6):452-8. doi: 10.1097/BRS.0b013e31823c8603.
Liu, H., Gomez, G., Lin, S., Lin, S. & Lin, C. Optogenetic control of transcription in zebrafish. PLoS One 7, e50738, doi: 10.1371/journal.pone.0050738 (2012).
Lunde, B. M., Magler, I. & Meinhart, A. Crystal structures of the Cid1 poly (U) polymerase reveal the mechanism for UTP selectivity. Nucleic Acids Res. 40, 9815-24 (2012).
Lv Y, Peng R, Zhou Y, Zhang X, Tan W. Catalytic self-assembly of a DNA dendritic complex for efficient gene silencing. Chem Commun (Camb). 2015 Dec. 2. [Epub ahead of print] PubMed PMID: 26626818.
Mart R J, Meah D, Allemann R K. Photocontrolled Exposure of Pro-apoptotic Peptide Sequences in LOV Proteins Modulates Bcl-2 Family Interactions. Chembiochem. 2016 Apr. 15; 17(8):698-701.
Masuda, Sh. and Bauer, C. E., AppA is a blue light photoreceptor that antire-presses photosynthesis gene expression in Rhodobacter sphaeroides, Cell 110, (2002), 613-623.
Meakin J R: Replacing the nucleus pulposus of the intervertebral disk: prediction of suitable properties of a replacement material using finite element analysis. J Mater Sci Mater Med 12: 207-213 (2001)
Morgan J L, Strumillo J, Zimmer J. Crystallographic snapshot of cellulose synthesis and membrane translocation. Nature. 2013 Jan. 10; 493(7431):181-6. doi:10.1038/nature11744.
Müller M, Bamann C, Bamberg E, Kühlbrandt W. Light-induced helix movements in channel rhodopsin-2. J Mol Biol. 2015 Jan. 30; 427(2):341-9.
Nobles jr., D. R. and Brown, jr. R. M. (2008) Transgenic expression of Gluconacetobacter xylinus Strain ATCC 53582 cellulose synthase genes in the cyanobacterium Synechococcus leopoliensis strain UTCC 100. Cellulose DOI 10.1007/s10570-008-9217-5
Okajima, K., Yoshihara, Sh., Fukushima, Y., Geng, X., Katayama, M., Higashi, Sh., Watanabe, M., Sato, Sh., Tabata, S., Shibata, Y., Itoh, Sh. and Ikeuchi, M., (2005) Biochemical and functional characterization of BLUF-type flavin-binding proteins of two species of cyanobacteria. J. Biochem. 137, 741-750.
Park, J. K., J. Y. Jung, and Y. H. Park (2003) Cellulose production by Gluconacetobacter hansenii in a medium containing ethanol. Biotechnol. Lett. 25: 2055-2059
Pesavento, C. and Hengge, R., Bacterial nucleotide-based second messengers. Current opinion in microbiology, 12(2), (2009), 170-176.
Pettis, Bre; France, Anna Kaziunas; Shergil J; Getting started with MakerBot 2012. Book Publisher: Maker Media, Inc.
Sakai D, Mochida J, Iwashina T, Watanabe T, Nakai T, Ando K, Hotta T. Differentiation of mesenchymal stem cells transplanted to a rabbit degenerative disc model: potential and limitations for stem cell therapy in disc regeneration. Spine (Phila Pa. 1976). 2005 Nov. 1; 30(20:2379-87.
Sakai D, Mochida J, Iwashina T, Hiyama A, Omi H, Imai M, Nakai T, Ando K, Hotta T. Biomaterials. Regenerative effects of transplanting mesenchymal stem cells embedded in atelocollagen to the degenerated intervertebral disc. 2006 January; 27(3):335-45.
Scheufens M. Eine neue industrielle Revolution? Spektrum der Wissenschaften Juni 2014; 92-95.
Sebastine Immanuel M., Williams David J. Current developments in Tissue Engineering of Nucleus Pulposus for Treatment of Invertebral Disc Degeneration. Conf Proc IEEE Eng Med Biol Soc. 2007; 2007: 6401-6.
Shetty, R. P., Endy, D. & Knight, T. F. Jr. Engineering BioBrick vectors from BioBrick parts. J. Biol. Eng. 14, 2-5 (2008).
Sirviö J A et al. Composite Films of Poly(vinyl alcohol) and Bifunctional Cross-linking Cellulose Nanocrystals. ACS Appl Mater Interfaces. 2015 Sep. 9; 7(35):19691-9. doi: 10.1021/acsami.5b04879. Epub 2015 Aug. 28.
Song, C. & Zhao, M. Real-time monitoring of the activity and kinetics of T4 polynucleotide kinase by a singly labeled DNA-hairpin smart probe coupled with lambda exonuclease cleavage. Analytical Chemistry 81, 1383-1388, doi:10.1021/ac802107w (2009).
Stratmann A T, Fecher D, Wangorsch G, Göttlich C, Walks T, Walles H, Dandekar T, Dandekar G, Nietzer S L. Establishment of a human 3D lung cancer model based on a biological tissue matrix combined with a Boolean in silico model. Mol Oncol. 2014 March; 8(2):351-65. doi: 10.1016/j.molonc.2013.11.009.
Tee B. C.-K., Wang, C., Allen, R. & Bao, Z. (2012). Nature Nanotechnology 7, 825-832 (2012) doi:10.1038/nnano.2012.192
Timper, J., Gutsmiedl, K., Wirges, C., Broda, J., Noyong, M., Mayer, J., Carell, T. & Simon, U. Herstellung leitfähiger Nanostrukturen durch Oberflächen-Klickreaktion and kontrollierte Metallisierung von DNA. Angewandte Chemie 124, 1-5 (2012).
Tschowri N, Busse S, Hengge R. The BLUF-EAL protein YcgF acts as a direct anti-repressor in a blue-light response of Escherichia coli. Genes Dev. 2009 Feb. 15; 23(4):522-34. doi: 10.1101/gad.499409. PubMed PMID: 19240136; PubMed Central PMCID: PMC2648647.
Tschowri N, Lindenberg S, Hengge R. Molecular function and potential evolution of the biofilm-modulating blue light-signalling pathway of Escherichia coli. Mol Microbiol. 2012 September; 85(5):893-906. doi: 10.1111/j.1365-2958.2012.08147.x. Epub 2012 Jul. 12. PubMed PMID: 22783906; PubMed Central PMCID: PMC3509220.
Tsourkas A, Behlke M A, Rose S D, Bao G. Hybridization kinetics and thermodynamics of molecular beacons. Nucleic Acids Res. 2003 Feb. 15; 31(4):1319-30. PubMed PMID: 12582252; PubMed Central PMCID: PMC150230.
Tyagi, A., Penzkofer, A., Griese, J., Schlichting, I., Kirienko, N. V., & Gomelsky, M. Photodynamics of blue-light-regulated phosphodiesterase BlrP1 protein from Klebsiella pneumoniae and its photoreceptor BLUF domain Chemical Physics, (2008). 354(1), 130-141.
Uchiyama, Y., Takeuchi, R., Kodera, H. & Sakaguchi, K. Distribution and roles of X-family DNA polymerases in eukaryotes. Biochimie 91, 165-70 (2009).
Vernengo J, Fussell G W, Smith N G, Lowman A M. Synthesis and characterization of injectable bioadhesive hydrogels for nucleus pulposus replacement and repair of the damaged intervertebral disc. J Biomed Mater Res B Appl Biomater. 2010 May; 93(2):309-17. doi: 10.1002/jbm.b.31547.
Wilke H J, Heuer F, Neidlinger-Wilke C, Claes L. Is a collagen scaffold for a tissue engineered nucleus replacement capable of restoring disc height and stability in an animal model? Eur Spine J. 2006 August; 15 Suppl 3: S433-8. Epub 2006 Jul. 26
Yao N, Lei M, Ren L, Menke N, Wang Y, et al. Polarization multiplexed write-once-read-many optical data storage in bacteriorhodopsin films. Opt Lett. 2005; 30:3060-3062.
Yao X, Rosen M K, Gardner K H. Estimation of the available free energy in a LOV2-J alpha photoswitch. Nat Chem Biol. 2008 August; 4(8):491-7. doi:10.1038/nchembio.99.
Yasukawa, H., Sato, A., Kita, A., Kodaira, K. I., Iseki, M., Takahashi, T., . . . & Yagita, K. Identification of photoactivated adenylyl cyclases in Naegleria australiensis and BLUF-containing protein in Naegleria fowleri. The Journal of general and applied microbiology, (2013) 59(5), 361-369.

1. A bacterial nanocellulose composite, said bacterial nanocellulose composite comprising nanocellulose and
(i) sensor or signal processing molecule(s);
and/or
(ii) actuator or effector molecule(s);
and/or
(iii) cells.

2. The bacterial nanocellulose composite of claim 1, wherein the bacterial nanocellulose is obtained via bacterial fermentation or bacterial expression of gram-negative bacteria, Komagataeibacter, cyanobacteria, or from plant sources but then bacterially fermented.

3. The bacterial nanocellulose composite of claim 1, comprising a light-inducible or light-responding sensor/actuator/effector molecule(s) or light-inducible or a light-responding sensor/actuator/effector domain(s) comprising:
blue light using FAD domain (BLUF domain),
light-oxygen voltage sensing domain (LOV domain),
or
cryptochromes (CRYs).

4. The bacterial nanocellulose composite of claim 1, wherein the sensor or signal processing molecule(s) (i) are protein(s) comprising light-inducible or light-responding sensor domains which are selected from:
polymerase(s);
adenyltransferase(s);
ion channel(s) or pore(s);
membrane protein(s), lipoprotein(s), glycoprotein(s);
receptors;
enzyme;
or domains thereof;
or combinations thereof.

5. The bacterial nanocellulose composite of claim 1, wherein the actuator or effector molecule(s) (ii) are proteins selected from
polymerase(s);
exonuclease(s);
transcription factor(s);
nucleotide binding domain(s);
enzyme(s);
structural protein(s);
protein translation enzyme(s);
or domains thereof,
or combinations thereof.

6. The bacterial nanocellulose composite of claim 3, wherein the protein(s) comprising light-inducible or light-responding sensor domain(s) further comprise linker(s) and/or secretion signal(s) or signal peptide domain(s).

7. The bacterial nanocellulose composite of claim 1, wherein sensor or signal processing molecule(s) (i) and/or the actuator or effector molecule(s) (ii) comprise or are fused to fluorescent protein(s) or protein domain(s) comprising fluorescent domain(s).

8. The bacterial nanocellulose composite of claim 1, wherein the bacterial nanocellulose further comprises
components for the sensor/actuator molecule(s) (i)
further polymer(s),
graphene or fullerene,
compounds supporting wound healing and/or stimulating growth,
markers or labels,
drugs, antibodies or antibody fragments,
or combinations thereof.

9. The bacterial nanocellulose composite of claim 8, wherein the sensor or signal processing molecule(s) (i) and/or actuator or effector molecule(s) (ii) and/or cell(s) (iii) and/or further component(s) (iv), if present, are embedded or encapsulated, or the sensor or signal processing molecule(s) (i) and/or actuator or effector molecule(s) (ii) and/or further component(s) (iv), if present, are covalently attached to the nanocellulose, such as via a linker, anchor groups or cantilever.

10. The bacterial nanocellulose composite of claim 1, wherein the nanocellulose comprises a surface or surface layer,
wherein said surface or surface layer comprises sensor or signal processing molecule(s) (i) selected from:
ion channel(s) or pore(s);
membrane protein(s), lipoprotein(s), glycoproteins;
receptor(s);
enzymes, which are preferably active on the surface;
or combinations thereof.

11. Use of a bacterial nanocellulose composite of claim 1
in material engineering,
in chip technology,
as printing matrix or printed nanocellulose composite,
as transparent material or display or information processing device for LED and chips/chip technology,
as printing, storage and/or processing medium,
as detector,
as intelligent foil,
as intelligent material,
as nanofactory,
as sophisticated, light-controlled, synthesis device,
as small biochemical analyzer,
in DNA-based ASIC (application-specific chip) for sequence storage or analysis
in wound healing and tissue engineering,
as skin transplant, band-aid or tissue implant,
as neuro transplant,
for stimulus conduction,
for muscle stimulation,
as electronic skin,
for monitoring wound healing, heartbeat, or other physical parameters,
for faster regeneration,
for reprogramming body cells during the healing process, or
as an intelligent plaster.

12. (canceled)

13. The use according to claim 11, wherein the bacterial nanocellulose composite is used in a form of a hydrogel, a foil, a layer, or optical transparent paper.

14. An article of manufacture comprising the bacterial nanocellulose composite of claim 1 wherein said article of manufacture is selected from a printing, storage and/or processing medium; a smart card or a chip card; a skin transplant; a tissue implant: a neuro transplant and electronic skin.

15-16. (canceled)

17. A method for treating a wound, detecting a wound and/or monitoring wound healing wherein said method comprises the use of the bacterial nanocellulose composite of claim 1.

18. A method for tissue engineering wherein said method comprises the use of the bacterial nanocellulose composite of claim 1.

19. (canceled)

20. A method for stimulus conduction, muscle stimulation and/or monitoring a heartbeat, wherein said method comprises the use of bacterial nanocellulose composite of claim 1.

21. (canceled)

22. A method for producing a nanocellulose composite chip, comprising the steps of
(1) providing a nanocellulose composite, preferably as defined in claim 1,
(2) using a 3D printer or laser sintering, and
(3) obtaining the nanocellulose composite chip.

23. The method of claim 22, wherein the nanocellulose in step (1) is bacterial nanocellulose, bacterial cellulose/poly caprolactone nanocomposite film, composite film of polyvinyl alcohol, bifunctional linking cellulose nanocrystals, or polylactide latex/nanofibrillated cellulose bio-nanocomposite,
and/or wherein the 3D printer in step (2) is an ink-jet printer, a sinter printer, or a printer with melt layering.

24. A nanocellulose composite chip obtained by the method of claim 22.


SEQUENCE LISTING (33 sequences)

SEQ ID NO: 1 - protein, 403 aa, Escherichia coli
MLTTLIYRSHIRDDEPVKKIEEMVSIANRRNMQSDVTGILLFNGSHFFQLLEGPEEQVKMIYRAICQDPRHYNIVELLCDYAPARRFGKAGMELFDLRLHERDDVLQAVFDKGTSKFQLTYDDRALQFFRTFVLATEQSTYFEIPAEDSWLFIADGSDKELDSCALSPTINDHFAFHPIVDPLSRRIIAFEAIVQKNEDSPSAIAVGQRKDGEIYTADLKSKALAFTMAHALELGDKMISINLLPMTLVNEPDAVSFLLNEIKANALVPEQIIVEFTESEVISRFDEFAEAIKSLKAAGISVAIDHFGAGFAGLLLLSRFQPDRIKISQELITNVHKSGPRQAIIQAIIKCCTSLEIQVSAMGVATPEEWMWLESAGIEMFQGDLFAKAKLNGIPSIAWPEKK

SEQ ID NO: 2 - protein, 84 aa, Escherichia coli
MLTTLIYRSHIRDDEPVKKIEEMVSIANRRNMQSDVTGILLFNGSHFFQLLEGPEEQVKMIYRAICQDPRHYNIVELLCDYAPA

SEQ ID NO: 3 - DNA, 252 bp, Escherichia coli
atgcttaccacccttatttatcgtagccatatacgtgacgacgaacctgtcaaaaaaatcgaagaaatggtttcgatagcaaatcgcaggaacatgcagtctgacgtaacagggatcttactgtttaatggttctcattttttccagcttctggaaggtccggaagaacaggttaaaatgatatatcgggctatatgccaggatccacggcactataatattgttgagctgctgtgcgattacgcgcctgct

SEQ ID NO: 4 - protein, 144 aa, Escherichia coli
MLTTLIYRSHIRDDEPVKKIEEMVSIANRRNMQSDVTGILLFNGSHFFQLLEGPEEQVKMIYRAICQDPRHYNIVELLCDYAPARRFGKAGMELFDLRLHERDDVLQAVFDKGTSKFQLTYDDRALQFFRTFVLATEQSTYFEI

SEQ ID NO: 5 - DNA, 435 bp, Escherichia coli
atgcttaccacccttatttatcgtagccatatacgtgacgacgaacctgtcaaaaaaatcgaagaaatggtttcgatagcaaatcgcaggaacatgcagtctgacgtaacagggatcttactgtttaatggttctcattttttccagcttctggaaggtccggaagaacaggttaaaatgatatatcgggctatatgccaggatccacggcactataatattgttgagctgctgtgcgattacgcgcctgctcgccgttttggcaaagcgggaatggaattatttgatttgcgcctgcacgagcgagatgacgttttacaggccgtattcgacaaaggcacatcaaaatttcagctaacttatgatgacagagcgctacaattttttcgtacttttgtccttgcaaccgaacaatcaacctatttcgagatctaa

SEQ ID NO: 6 - protein, 125 aa, Escherichia coli
MLTTLIYRSHIRDDEPVKKIEEMVSIANRRNMQSDVTGILLFNGSHFFQLLEGPEEQVKMIYRAICQDPRHYNIVELLCDYAPARRFGKAGMELFDLRLHERDDVLQAVFDKGTSKFQLTYDDRA

SEQ ID NO: 7 - DNA, 375 bp, Escherichia coli
atgcttaccacccttatttatcgtagccatatacgtgacgacgaacctgtcaaaaaaatcgaagaaatggtttcgatagcaaatcgcaggaacatgcagtctgacgtaacagggatcttactgtttaatggttctcattttttccagcttctggaaggtccggaagaacaggttaaaatgatatatcgggctatatgccaggatccacggcactataatattgttgagctgctgtgcgattacgcgcctgctcgccgttttggcaaagcgggaatggaattatttgatttgcgcctgcacgagcgagatgacgttttacaggccgtattcgacaaaggcacatcaaaatttcagctaacttatgatgacagagcg

SEQ ID NO: 8 - protein, 130 aa, Escherichia coli
FPRVSQELKTALSTLQQTFVVSDATQPHCPIVYASSGFFTMTGYSSKEIVGRNCRFLQGPDTDKNEVAKIRDCVKNGKSYCGRLLNYKKDGTPFWNLLTVTPIKDDQGNTIKFIGMQVEVSKYTEGVNDK

SEQ ID NO: 9 - protein, 405 aa, Schizosaccharomyces pombe
MNISSAQFIPGVHTVEEIEAEIHKNLHISKSCSYQKVPNSHKEFTKFCYEVYNEIKISDKEFKEKRAALDTLRLCLKRISPDAELVAFGSLESGLALKNSDMDLCVLMDSRVQSDTIALQFYEELIAEGFEGKFLQRARIPIIKLTSDTKNGFGASFQCDIGFNNRLAIHNTLLLSSYTKLDARLKPMVLLVKHWAKRKQINSPYFGTLSSYGYVLMVLYYLIHVIKPPVFPNLLLSPLKQEKIVDGFDVGFDDKLEDIPPSQNYSSLGSLLHGFFRFYAYKFEPREKVVTFRRPDGYLTKQEKGWTSATEHTGSADQIIKDRYILAIEDPFEISHNVGRTVSSSGLYRIRGEFMAASRLLNSRSYPIPYDSLFEEAPIPPRRQKKTDEQSNKKLLNETDGDNSE

SEQ ID NO: 10 - protein, 373 aa, Schizosaccharomyces pombe
SYQKVPNSHKEFTKFCYEVYNEIKISDKEFKEKRAALDTLRLCLKRISPDAELVAFGSLESGLALKNSDMDLCVLMDSRVQSDTIALQFYEELIAEGFEGKFLQRARIPIIKLTSDTKNGFGASFQCDIGFNNRLAIHNTLLLSSYTKLDARLKPMVLLVKHWAKRKQINSPYFGTLSSYGYVLMVLYYLIHVIKPPVFPNLLLSPLKQEKIVDGFDVGFDDKLEDIPPSQNYSSLGSLLHGFFRFYAYKFEPREKVVTFRRPDGYLTKQEKGWTSATEHTGSADQIIKDRYILAIEDPFEISHNVGRTVSSSGLYRIRGEFMAASRLLNSRSYPIPYDSLFEEAPIPPRRQKKTDEQSNKKLLNETDGDNSE

SEQ ID NO: 11 - DNA, 1122 bp, Schizosaccharomyces pombe
agctaccaaaaggtccctaattcgcacaaggaatttacgaagttttgctatgaagtgtataatgagattaaaattagtgacaaagagtttaaagaaaagagagcggcattagatacacttcggctatgccttaaacgaatatcccctgatgctgaattggtagcctttggaagtttggaatctggtttagcacttaaaaattcggatatggatttgtgcgtgcttatggattcgcgcgtccaaagtgatacaattgcgctccaattctatgaagagcttatagctgaaggatttgaaggaaaatttttacaaagggcaagaattcccattatcaaattaacatctgatacgaaaaatggatttggggcttcgtttcaatgtgatattggatttaacaatcgtctagctattcataatacgcttttactttcttcatatacaaaattagatgctcgcctaaaacccatggtccttcttgttaagcattgggccaaacggaagcaaatcaactctccttactttggaactctttccagttatggttacgtcctaatggttctttactatctgattcacgttatcaagcctcccgtctttcctaatttactgttgtcacctttgaaacaagaaaagatagttgatggatttgacgttggttttgacgataaactggaagatatccctccttcccaaaattatagctcattgggaagtttacttcatggcttttttagattttatgcttataagttcgagccacgggaaaaggtagtaacttttcgtagaccagacggttacctcacaaagcaagagaaaggatggacttcagctactgaacacactggatcggctgatcaaattataaaagacaggtatattcttgcgattgaagatcctttcgagatttcacataatgtgggtaggacagttagcagttctggattgtatcggattcgaggggaatttatggccgcttcaaggttgctcaattctcgctcatatcctatcccttatgattcattatttgaggaggccccaattccgcctcgtcgccagaaaaaaacggatgaacaatctaacaaaaaattgttgaatgaaaccgatggtgacaattctgagtga

SEQ ID NO: 12 - protein, 377 aa, Schizosaccharomyces pombe
MNISSAQFIPGVHTVEEIEAEIHKNLHISKSCSYQKVPNSHKEFTKFCYEVYNEIKISDKEFKEKRAALDTLRLCLKRISPDAELVAFGSLESGLALKNSDMDLCVLMDSRVQSDTIALQFYEELIAEGFEGKFLQRARIPIIKLTSDTKNGFGASFQCDIGFNNRLAIHNTLLLSSYTKLDARLKPMVLLVKHWAKRKQINSPYFGTLSSYGYVLMVLYYLIHVIKPPVFPNLLLSPLKQEKIVDGFDVGFDDKLEDIPPSQNYSSLGSLLHGFFRFYAYKFEPREKVVTFRRPDGYLTKQEKGWTSATEHTGSADQIIKDRYILAIEDPFEISHNVGRTVSSSGLYRIRGEFMAASRLLNSRSYPIPYDSLFEEA

SEQ ID NO: 13 - DNA, 1131 bp, Schizosaccharomyces pombe
atgaacatttcttctgcacaatttattcctggtgttcacacagttgaagagattgaggcagaaattcacaaaaatttacatatttcaaaaagttgtagctaccaaaaggtccctaattcgcacaaggaatttacgaagttttgctatgaagtgtataatgagattaaaattagtgacaaagagtttaaagaaaagagagcggcattagatacacttcggctatgccttaaacgaatatcccctgatgctgaattggtagcctttggaagtttggaatctggtttagcacttaaaaattcggatatggatttgtgcgtgcttatggattcgcgcgtccaaagtgatacaattgcgctccaattctatgaagagcttatagctgaaggatttgaaggaaaatttttacaaagggcaagaattcccattatcaaattaacatctgatacgaaaaatggatttggggcttcgtttcaatgtgatattggatttaacaatcgtctagctattcataatacgcttttactttcttcatatacaaaattagatgctcgcctaaaacccatggtccttcttgttaagcattgggccaaacggaagcaaatcaactctccttactttggaactctttccagttatggttacgtcctaatggttctttactatctgattcacgttatcaagcctcccgtctttcctaatttactgttgtcacctttgaaacaagaaaagatagttgatggatttgacgttggttttgacgataaactggaagatatccctccttcccaaaattatagctcattgggaagtttacttcatggcttttttagattttatgcttataagttcgagccacgggaaaaggtagtaacttttcgtagaccagacggttacctcacaaagcaagagaaaggatggacttcagctactgaacacactggatcggctgatcaaattataaaagacaggtatattcttgcgattgaagatcctttcgagatttcacataatgtgggtaggacagttagcagttctggattgtatcggattcgaggggaatttatggccgcttcaaggttgctcaattctcgctcatatcctatcccttatgattcattatttgaggaggcc

SEQ ID NO: 14 - protein, 331 aa, Schizosaccharomyces pombe
MNISSAQFIPGVHTVEEIEAEIHKNLHISKSCSYQKVPNSHKEFTKFCYEVYNEIKISDKEFKEKRAALDTLRLCLKRISPDAELVAFGSLESGLALKNSDMDLCVLMDSRVQSDTIALQFYEELIAEGFEGKFLQRARIPIIKLTSDTKNGFGASFQCDIGFNNRLAIHNTLLLSSYTKLDARLKPMVLLVKHWAKRKQINSPYFGTLSSYGYVLMVLYYLIHVIKPPVFPNLLLSPLKQEKIVDGFDVGFDDKLEDIPPSQNYSSLGSLLHGFFRFYAYKFEPREKVVTFRRPDGYLTKQEKGWTSATEHTGSADQIIKDRYILAIEDP

SEQ ID NO: 15 - DNA, 993 bp, Schizosaccharomyces pombe
atgaacatttcttctgcacaatttattcctggtgttcacacagttgaagagattgaggcagaaattcacaaaaatttacatatttcaaaaagttgtagctaccaaaaggtccctaattcgcacaaggaatttacgaagttttgctatgaagtgtataatgagattaaaattagtgacaaagagtttaaagaaaagagagcggcattagatacacttcggctatgccttaaacgaatatcccctgatgctgaattggtagcctttggaagtttggaatctggtttagcacttaaaaattcggatatggatttgtgcgtgcttatggattcgcgcgtccaaagtgatacaattgcgctccaattctatgaagagcttatagctgaaggatttgaaggaaaatttttacaaagggcaagaattcccattatcaaattaacatctgatacgaaaaatggatttggggcttcgtttcaatgtgatattggatttaacaatcgtctagctattcataatacgcttttactttcttcatatacaaaattagatgctcgcctaaaacccatggtccttcttgttaagcattgggccaaacggaagcaaatcaactctccttactttggaactctttccagttatggttacgtcctaatggttctttactatctgattcacgttatcaagcctcccgtctttcctaatttactgttgtcacctttgaaacaagaaaagatagttgatggatttgacgttggttttgacgataaactggaagatatccctccttcccaaaattatagctcattgggaagtttacttcatggcttttttagattttatgcttataagttcgagccacgggaaaaggtagtaacttttcgtagaccagacggttacctcacaaagcaagagaaaggatggacttcagctactgaacacactggatcggctgatcaaattataaaagacaggtatattcttgcgattgaagatcct

SEQ ID NO: 16 - protein, 74 aa, Schizosaccharomyces pombe
FEISHNVGRTVSSSGLYRIRGEFMAASRLLNSRSYPIPYDSLFEEAPIPPRRQKKTDEQSNKKLLNETDGDNSE

SEQ ID NO: 17 - DNA, 225 bp, Schizosaccharomyces pombe
ttcgagatttcacataatgtgggtaggacagttagcagttctggattgtatcggattcgaggggaatttatggccgcttcaaggttgctcaattctcgctcatatcctatcccttatgattcattatttgaggaggccccaattccgcctcgtcgccagaaaaaaacggatgaacaatctaacaaaaaattgttgaatgaaaccgatggtgacaattctgagtga

SEQ ID NO: 18 - protein, 15 aa, Artificial Sequence
  Linker
GGGGSGGGGSGGGGS

SEQ ID NO: 19 - DNA, 45 bp, Artificial Sequence
  Linker
ggcggaggaggtagcggtggcggagggtcaggtggtgggggaagt

SEQ ID NO: 20 - protein, 301 aa, enterobacteria phage T4
MKKIILTIGCPGSGKSTWAREFIAKNPGFYNINRDDYRQSIMAHEERDEYKYTKKKEGIVTGMQFDTAKSILYGGDSVKGVIISDTNLNPERRLAWETFAKEYGWKVEHKVFDVPWTELVKRNSKRGTKAVPIDVLRSMYKSMREYLGLPVYNGTPGKPKAVIFDVDGTLAKMNGRGPYDLEKCDTDVINPMVVELSKMYALMGYQIVVVSGRESGTKEDPTKYYRMTRKWVEDIAGVPLVMQCQREQGDTRKDDVVKEEIFWKHIAPHFDVKLAIDDRTQVVEMWRRIGVECWQVASGDF

SEQ ID NO: 21 - DNA, 45 bp, Artificial Sequence
  Primer
aaaaaactgcagagatctatgcttaccacccttatttatcgtagc

SEQ ID NO: 22 - DNA, 45 bp, Artificial Sequence
  Primer
ttttttgagctcttcgaagcgagacagtagtattcaatcgacttt

SEQ ID NO: 23 - DNA, 28 bp, Artificial Sequence
  Beacon MB1
cctcgtgtcttgtacttcccgtccgagg

SEQ ID NO: 24 - DNA, 19 bp, Artificial Sequence
  Oligo A
gacgggaagtacaagacac

SEQ ID NO: 25 - DNA, 30 bp, Artificial Sequence
  Beacon MB_2_Poly-U
cctcaaaaaaaaaaaaaaacgcggctgagg

SEQ ID NO: 26 - RNA, 21 nt, Artificial Sequence
  Oligo_PUr
gccgcguuuuuuuuuuuuuuu

SEQ ID NO: 27 - RNA, 10 nt, Artificial Sequence
  Oligo_PriUr
gccgcguuuu

SEQ ID NO: 28 - DNA, 30 bp, Artificial Sequence
  Primer
tatggatccccggtcaacgcgcggcttatc

SEQ ID NO: 29 - DNA, 30 bp, Artificial Sequence
  Primer
tctgtcgacagccaaagcctgatccgatgg

SEQ ID NO: 30 - DNA, 30 bp, Artificial Sequence
  Primer
cctggtaccgcaacgcaaccactgatcaat

SEQ ID NO: 31 - DNA, 30 bp, Artificial Sequence
  Primer
atgctcagcatccgggttaagacgacgacg

SEQ ID NO: 32 - protein, 872 aa, Escherichia coli
MSILTRWLLIPPVNARLIGRYRDYRRHGASAFSATLGCFWMILAWIFIPLEHPRWQRIRAEHKNLYPHINASRPRPLDPVRYLIQTCWLLIGASRKETPKPRRRAFSGLQNIRGRYHQWMNELPERVSHKTQHLDEKKELGHLSAGARRLILGIIVTFSLILALICVTQPFNPLAQFIFLMLLWGVALIVRRMPGRFSALMLIVLSLTVSCRYIWWRYTSTLNWDDPVSLVCGLILLFAETYAWIVLVLGYFQVVWPLNRQPVPLPKDMSLWPSVDIFVPTYNEDLNVVKNTIYASLGIDWPKDKLNIWILDDGGREEFRQFAQNVGVKYIARTTHEHAKAGNINNALKYAKGEFVSIFDCDHVPTRSFLQMTMGWFLKEKQLAMMQTPHHFFSPDPFERNLGRFRKTPNEGTLFYGLVQDGNDMWDATFFCGSCAVIRRKPLDEIGGIAVETVTEDAHTSLRLHRRGYTSAYMRIPQAAGLATESLSAHIGQRIRWARGMVQIFRLDNPLTGKGLKFAQRLCYVNAMFHFLSGIPRLIFLTAPLAFLLLHAYIIYAPALMIALFVLPHMIHASLTNSKIQGKYRHSFWSEIYETVLAWYIAPPTLVALINPHKGKFNVTAKGGLVEEEYVDWVISRPYIFLVLLNLVGVAVGIWRYFYGPPTEMLTVVVSMVWVFYNLIVLGGAVAVSVESKQVRRSHRVEMTMPAAIAREDGHLFSCTVQDFSDGGLGIKINGQAQILEGQKVNLLLKRGQQEYVFPTQVARVMGNEVGLKLMPLTTQQHIDFVQCTFARADTWALWQDSYPEDKPLESLLDILKLGFRGYRHLAEFAPSSVKGIFRVLTSLVSWVVSFIPRRPERSETAQPSDQALAQQ

SEQ ID NO: 33 - protein, 779 aa, Escherichia coli
MKRKLFWICAVAMGMSAFPSFMTQATPATQPLINAEPAVAAQTEQNPQVGQVMPGVQGADAPVVAQNGPSRDVKLTFAQIAPPPGSMVLRGINPNGSIEFGMRSDEVVTKAMLNLEYTPSPSLLPVQSQLKVYLNDELMGVLPVTKEQLGKKTLAQMPINPLFISDFNRVRLEFVGHYQDVCEKPASTTLWLDVGRSSGLDLTYQTLNVKNDLSHFPVPFFDPSDNRTNTLPMVFAGAPDVGLQQASAIVASWFGSRSGWRGQNFPVLYNQLPDRNAIVFATNDKRPDFLRDHPAVKAPVIEMINHPQNPYVKLLVVFGRDDKDLLQAAKGIAQGNILFRGESVVVNEVKPLLPRKPYDAPNWVRTDRPVTFGELKTYEEQLQSSGLEPAAINVSLNLPPDLYLMRSTGIDMDINYRYTMPPVKDSSRMDISLNNQFLQSFNLSSKQEANRLLLRIPVLQGLLDGKTDVSIPALKLGATNQLRFDFEYMNPMPGGSVDNCITFQPVQNHVVIGDDSTIDFSKYYHFIPMPDLRAFANAGFPFSRMADLSQTITVMPKAPNEAQMETLLNTVGFIGAQTGFPAINLTVTDDGSTIQGKDADIMIIGGIPDKLKDDKQIDLLVQATESWVKTPMRQTPFPGIVPDESDRAAETRSTLTSSGAMAAVIGFQSPYNDQRSVIALLADSPRGYEMLNDAVNDSGKRATMFGSVAVIRESGINSLRVGDVYYVGHLPWFERVWYALANHPILLAVLAAISVILLAWVLWRLLRIISRRRLNPDNE